(19) United States
(12) Patent Application Publication
(10) Pub. No.: US 2013/0137628 A1
(43) Pub. Date: May 30, 2013

(54) Dimeric Oxidation-Resistant Apolipoprotein A1 Variants

(71) Applicants: Roger S. Newton, Ann Arbor, MI (US); Rai Ajit K. Srivastava, Churchville, PA (US); Timothy R. Hurley, Ann Arbor, MI (US); Clay Cramer, Dexter, MI (US); Sergey V. Filippov, Canterbury, CT (US); Stephen L. Pinkosky, Plymouth, MI (US)

(72) Inventors: Roger S. Newton, Ann Arbor, MI (US); Rai Ajit K. Srivastava, Churchville, PA (US); Timothy R. Hurley, Ann Arbor, MI (US); Clay Cramer, Dexter, MI (US); Sergey V. Filippov, Canterbury, CT (US); Stephen L. Pinkosky, Plymouth, MI (US)

(73) Assignee: ESPERION THERAPEUTICS, INC., Plymouth, MI (US)

(21) Appl. No.: 13/697,251

(22) PCT Filed: May 11, 2011
(86) PCT No.: PCT/US11/36144

§ 371 Date: Feb. 12, 2013

Related U.S. Application Data
(60) Provisional application No. 61/333,454, filed on May 11, 2010.

(51) Int. Cl. C07K 14/775

(52) U.S. Cl. CPC C07K 14/775
USPC 514/1.9; 530/359; 514/1.1; 514/21.2; 514/17.8; 514/19.3; 514/19.5; 514/19.4; 514/15.4; 514/20.8; 514/6.8; 514/15.7; 514/16.6; 514/4.8; 514/17.7; 514/7.4; 514/16.4; 435/375

(57) ABSTRACT

An isolated oxidation-resistant ApoA1 variant dimer includes a first oxidation-resistant ApoA1 variant polypeptide monomer and a second oxidation-resistant ApoA1 variant polypeptide monomer, wherein at least one of the first and the second monomers comprises at least one amino acid substitution of a tryptophan residue for an oxidation resistant amino acid, or a functional fragment or variant thereof. Methods for treating a disease or disorder comprises administering to a subject in need thereof, a therapeutically effective amount of an isolated oxidation-resistant ApoA1 variant dimer, an oxidation-resistant ApoA1 variant monomer, an oxidation-resistant ApoA1 monomer-lipid complex, a lipid complexed oxidation-resistant ApoA1 variant monomer, a lipid complexed oxidation-resistant ApoA1 variant dimer, or combinations thereof to the subject to enhance cholesterol efflux activity in the presence of an oxidant.

### CROSS-REFERENCE TO RELATED APPLICATION

This application claims the benefit of U.S. Provisional Application No. 61/333,454, filed on May 11, 2010, the content of which is hereby incorporated by reference in its entirety.

### FIELD

The present invention relates to dimeric oxidation-resistant variants of apolipoprotein A1, pharmaceutical compositions and formulations comprising the same and their uses in the treatment of disease.

### BACKGROUND

Apolipoprotein A1 (ApoA1) is the major component of high-density lipoprotein (HDL), popularly known as the “good cholesterol.” High plasma levels of ApoA1 are known to be associated with reduced risk of coronary heart disease (CHD) and presence of coronary lesions (Maciejko et al., N. Engl. J. Med. (1983), 309:385-389, Sedlis et al., Circulation (1986) 73:978-984). Plasma ApoA1 is a single polypeptide chain of 243 amino acids (Brewer et al., Biochem. Biophys. Res. Commun. (1978) 80:623 630). ApoA1 is synthesized as a 267 amino acid precursor in the cell. This pre-pro apolipoprotein is processed by N-terminal cleavage first intracellularly where 18 amino acids are lost and then with a further cleavage of 6 amino acids in the plasma or the lymph by the activity of specific proteases. The major structural requirement of the ApoA1 molecule is believed to be the presence of repeat units of 18 or 22 amino acids, presumed to exist in amphipathic helical conformation (Segrest et al., FEBS Lett (1974) 38:247-253). This structure allows for the main biological activities of ApoA1, i.e. lipid binding and lecithin cholesterol acyl transferase (LCAT) activation, anti-inflammatory, anti-platelet, anti-thrombotic, anti-oxidant, and anti-apoptotic activities.

The process of removing excess cholesterol from peripheral tissues has been termed reverse cholesterol transport (RCT), and cellular cholesterol efflux is the first step in RCT (Brewer HB. High-Density Lipoproteins: A New Potential Therapeutic Target for the Prevention of Cardiovascular Disease, Arterioscler Thromb Vasc Biol. (2004) 24: 387-391. 2. Rader DJ. Regulation of reverse cholesterol transport and clinical implications. Am J Cardiol. (2004) 92: 42J-49J). A number of different pathways have been identified by which unesterified/free cholesterol (FC) from cells is removed on incubation with apolipoproteins, such as apoAI. A number of active pathways that are linked to the presence of specific efflux proteins have been identified. These proteins include scavenger receptor class B type I (SR-BI) (Krieger M. Scavenger receptor class B type I is a multiligand HDL receptor that influences diverse physiologic systems, J Clin Invest. (2001) 108: 793-797) and a range of ATP-binding cassette (ABC) transporters, ABCA1, ABCG1, and ABCG4. SR-BI preferentially mediates the flux of FC to larger phospholipid-rich high-density lipoproteins (HDL), whereas ABCA1 prefers small preB-HDL and lipid-poor apolipoproteins (apo) such as apoA-I. Lipid-depleted ApoA1 removes excess cholesterol and phospholipids from cells such as macrophages through its interaction with a cell membrane protein called ATP-binding cassette transporter A1 (ABCA1). This process has broad specificity for multiple exchangeable HDL apolipoproteins, and is believed to be the rate-limiting step in the generation of mature HDL particles. Studies of human patients and animal models have shown that ABCA1 is cardioprotective. For example, loss-of-function mutations in human ABCA1 cause a severe HDL deficiency syndrome characterized by deposition of cholesterol in tissue macrophages and prevalent cardiovascular disease (CVD). Deletion of the ABCA1 gene in mouse macrophages increases atherosclerosis, and increasing ABCA1 expression in mice decreases atherosclerosis. The interaction of ApoA1 with ABCA1 or ABCA1-expressing cells elicits several responses involved in exporting cellular cholesterol: removing cholesterol and phospholipids that are transported to the cell surface by ABCA1, stabilizing ABCA1 protein so that it has sustained activity, and stimulating cellular signaling pathways that control ABCA1 activity. Gene transcription of ABCA1 is highly induced by cellular cholesterol.

Recently, intense interest has developed in using HDL or ApoA1 therapeutics to treat or prevent cardiovascular disease. However, there is a need to provide variants and mimetic ApoA1 proteins that have improved or sustained activity in-vivo. Improved or sustained activity can result from increased pharmacokinetic parameters that enable ApoA1 to remain in circulation longer, and not be degraded or otherwise impaired in its ability to efflux cholesterol from lipid laden cells.

The statements in this section merely provide background information related to the present disclosure and may not constitute prior art.

### SUMMARY

The present invention relates to ApoA1 variant constructs that are dimeric in form, affording enhanced cholesterol efflux from lipid-laden cells even in the presence of oxidizing agents. In a first aspect, the invention relates to an isolated oxidation-resistant ApoA1 variant dimer comprising a first oxidation-resistant ApoA1 variant polypeptide monomer and a second oxidation-resistant ApoA1 variant polypeptide monomer. At least one of the first oxidation-resistant ApoA1 variant polypeptide monomer and the second oxidation-resistant ApoA1 variant polypeptide monomer comprises at least one amino acid substitution of a tryptophan residue for an oxidation resistant amino acid. Also included are dimers comprising functional fragments or variants thereof.

In another aspect, the present invention provides pharmaceutical compositions and formulations comprising an oxidation-resistant ApoA1 variant dimer having a first oxidation-resistant ApoA1 variant polypeptide monomer and a second oxidation-resistant ApoA1 variant polypeptide monomer, wherein the first oxidation-resistant ApoA1 variant polypeptide monomer and the second oxidation-resistant ApoA1 variant polypeptide monomer of the dimer comprises at least one amino acid substitution of a tryptophan residue for an oxidation resistant amino acid, or a functional fragment or variant thereof. In another related aspect, the pharmaceutical composition includes lipid complexes, for example, a phospholipid complex comprising, at least one of an oxidation-resistant ApoA1 variant monomer, or an oxidation-resistant ApoA1 variant dimer, or an oxidation-resistant ApoA1 monomer, and a phosphiolipid.

In a further aspect, the present invention also relates to methods for treating a variety of diseases, which include: Alzheimer's disease, cancer, for example, prostate cancer, breast cancer or colon cancer, cardiovascular diseases, diabetic nephropathy, diabetic retinopathy, disorders of glucose metabolism, dyslipidemia, dyslipoproteinemia, hypertension, impotence, inflammation, rheumatoid arthritis, insulin resistance disorders, lipid elimination in bile, obesity, oxysterol elimination in bile, pancreatitis (pancreatitus) Parkinson's disease, peroxisome proliferator activated receptor (PPAR)-associated disorders, phospholipid elimination in bile, renal diseases, septicemia, metabolic syndrome disorders, thrombotic disorders, C-reactive protein imbalance and insufficient bile production by administering a therapeutically effective amount of a composition comprising an oxidation-resistant ApoA1 monomer or dimer, or an oxidation-resistant ApoA1 monomer-phospholipid complex, or a naked oxidation-resistant ApoA1 variant monomer or dimer.

The isolated oxidation-resistant ApoA1 variant dimers have at least one of the tryptophan amino acid residues substituted with at least one oxidation resistant amino acid. In this aspect, exemplary oxidation-resistant amino acids include leucine, alanine, valine, isoleucine, and phenylalanine.

Further areas of applicability will become apparent from the description provided herein. It should be understood that the description and specific examples are intended for purposes of illustration only and are not intended to limit the scope of the present disclosure.

### DRAWINGS

The drawings described herein are for illustration purposes only and are not intended to limit the scope of the present disclosure in any way.

FIG. 1 depicts the sequence listings of: human ApoA1-Milano (SEQ ID NO:1); human ApoA1-Paris (SEQ ID NO:2); generic human oxidation-resistant variant polypeptide monomer based on sequence of human ApoA1-Milano (SEQ ID NO:3); and generic human oxidation-resistant variant polypeptide monomer based on sequence of human ApoA1-Paris (SEQ ID NO:4) and mature 243 amino acid human oxidation-resistant ApoA1, also referred to as 4WF (SEQ ID NO:35).

FIG. 2A depicts the amino acid sequences of oxidation-resistant polypeptide monomers based on ApoA1 variant polypeptide monomer of SEQ ID NO:3.

FIG. 2B depicts the amino acid sequences of oxidation-resistant polypeptide monomers based on ApoA1 variant polypeptide monomer of SEQ ID NO:4.

FIG. 2C depicts the polynucleotide sequence of an exemplary oxidation-resistant polypeptide monomer based on ApoA1 variant polypeptide monomer of SEQ ID NO:5.

FIG. 3 depicts photomicrographs of two SDS-PAGE electropherograms. Human purified ApoA1 (hA-I), recombinant wild-type ApoA1 (WT A-I), 4WF and OxResD dimer (Dimer of monomers of SEQ ID NO:5) were subjected to oxidative damage with MPO/HOCL axis and protein modifications were assessed under reduced (FIG. 3A) and non-reduced (FIG. 3B) conditions. Arrows indicate multiple high molecular weight species of ApoA-I variants after MPO-mediated damage. Selected area shows OxResD dimer.

FIG. 4 depicts a bar chart illustrating ABCA1-dependent cholesterol efflux potential of native vs. modified ApoA1 variants.

### DETAILED DESCRIPTION

The following description is merely exemplary in nature and is not intended to limit the present disclosure, application, or uses.

The following description of technology is merely exemplary in nature of the subject matter, manufacture and use of one or more inventions, and is not intended to limit the scope, application, or uses of any specific invention claimed in this application or in such other applications as may be filed claiming priority to this application, or patents issuing therefrom. The following definitions and non-limiting guidelines must be considered in reviewing the description of the technology set forth herein.

The headings (such as “Background” and “Summary”) and sub-headings used herein are intended only for general organization of topics within the present invention, and are not intended to limit the disclosure of the present invention or any aspect thereof. In particular, subject matter disclosed in the “Background” may include novel technology and may not constitute a recitation of prior art. Subject matter disclosed in the “Summary” is not an exhaustive or complete disclosure of the entire scope of the technology or any embodiments thereof. Classification or discussion of a material within a section of this specification as having a particular utility is made for convenience, and no inference should be drawn that the material must necessarily or solely function in accordance with its classification herein when it is used in any given composition.

The citation of references herein does not constitute an admission that those references are prior art or have any relevance to the patentability of the technology disclosed herein. Any discussion of the content of references cited in the Introduction is intended merely to provide a general summary of assertions made by the authors of the references, and does not constitute an admission as to the accuracy of the content of such references. All references cited in the “Description” section of this specification are hereby incorporated by reference in their entirety.

The description and specific examples, while indicating embodiments of the technology, are intended for purposes of illustration only and are not intended to limit the scope of the technology. Moreover, recitation of multiple embodiments having stated features is not intended to exclude other embodiments having additional features, or other embodiments incorporating different combinations of the stated features. Specific examples are provided for illustrative purposes of how to make and use the compositions and methods of this technology and, unless explicitly stated otherwise, are not intended to be a representation that given embodiments of this technology have, or have not, been made or tested.

As used herein, the words “preferred” and “preferably” refer to embodiments of the technology that afford certain benefits, under certain circumstances. However, other embodiments may also be preferred, under the same or other circumstances. Furthermore, the recitation of one or more preferred embodiments does not imply that other embodiments are not useful, and is not intended to exclude other embodiments from the scope of the technology.

As referred to herein, all compositional percentages are by weight of the total composition, unless otherwise specified. As used herein, the word “include,” and its variants, is intended to be non-limiting, such that recitation of items in a list is not to the exclusion of other like items that may also be useful in the materials, compositions, devices, and methods of this technology. Similarly, the terms “can” and “may” and their variants are intended to be non-limiting, such that recitation that an embodiment can or may comprise certain elements or features does not exclude other embodiments of the present invention that do not contain those elements or features.

Disclosure of values and ranges of values for specific parameters (such as temperatures, molecular weights, weight percentages, etc.) are not exclusive of other values and ranges of values useful herein. It is envisioned that two or more specific exemplified values for a given parameter may define endpoints for a range of values that may be claimed for the parameter. For example, if Parameter X is exemplified herein to have value A and also exemplified to have value Z, it is envisioned that parameter X may have a range of values from about A to about Z. Similarly, it is envisioned that disclosure of two or more ranges of values for a parameter (whether such ranges are nested, overlapping or distinct) subsume all possible combination of ranges for the value that might be claimed using endpoints of the disclosed ranges. For example, if parameter X is exemplified herein to have values in the range of 1-10 it is also envisioned that Parameter X may have other ranges of values including 1-9, 2-9, 3-8, 1-8, 1-3, 1-2, 2-10, 2.5-7.8, 2-8, 2-3, 3-10, and 3-9.

Although the open-ended term “comprising,” as a synonym of terms such as including, containing, or having, is use herein to describe and claim the present invention, the invention, or embodiments thereof, may alternatively be described using more limiting terms such as “consisting of” or “consisting essentially of” the recited ingredients.

“Identity” or “similarity”, as known in the art, refers to relationships between two or more polypeptide sequences or two or more polynucleotide sequences, as determined by comparing the sequences. In the art, identity also means the degree of sequence relatedness between polypeptide or polynucleotide sequences, as the case may be, as determined by the match between strings of such sequences. Both identity and similarity can be readily calculated by known methods such as those described in: Computational Molecular Biology, Lesk, A. M., ed., Oxford University Press, New York, (1988); Biocomputing: Informatics and Genome Projects, Smith, D. W., ed., Academic Press, New York, (1993); Sequence Analysis in Molecular Biology, von Heinje, G., Academic Press, (1987); Computer Analysis of Sequence Data, Part I, Griffin, A. M., and Griffin, H. G., eds., Humana Press, New Jersey, (1994); and Sequence Analysis Primer, Gribskov, M. and Devereux, J., eds., M Stockton Press, New York, (1991). Methods commonly employed to determine identity or similarity between sequences includes, but are not limited to those disclosed in Carillo, H. and Lipman, D., SIAM J. Applied Math. 48:1073 (1988). Methods to determine identity and similarity are codified in publicly available computer programs. Computer program methods to determine identity and similarity between two or more sequences include, but are not limited to, GCG program package (Devereux, J., et al., Nucleic Acids Res. (1984) 12(1):387), BLASTP, BLASTN, and FASTA (Paschal, S. F. et al., J. Molec. Biol. (1990) 215: 403).

The term “homologous” refers to the degree of sequence similarity between two polymers (i.e., polypeptide molecules or nucleic acid molecules). The homology percentage figures referred to herein reflect the maximal homology possible between the two polymers, i.e., the percent homology when the two polymers are so aligned as to have the greatest number of matched (homologous) positions.

The term “percent homology” refers to the extent of amino acid or nucleotide sequence identity between polypeptides and polynucleotides respectively. The homology between any two polypeptides or polynucleotides is a direct function of the total number of matching amino acids or nucleotides at a given position in either sequence, e.g., if half of the total number of amino acids or polynucleotides in either of the corresponding sequences is the same then the two sequences are said to exhibit 50% homology.

The term “ortholog” refers to genes or proteins that are homologs via speciation, e.g., closely related and assumed to have common descent based on structural and functional considerations. Orthologous proteins generally have the same function and the same activity in different species. The term “paralog” refers to genes or proteins that are homologs via gene duplication, e.g., duplicated variants of a gene within a genome. See also, Fritch, W M Syst. Zool. (1970) 19:99-113. The term “ortholog” may refer to a polypeptide from another species that corresponds to human dimeric oxidation-resistant ApoA1 variant polypeptide amino acid sequence as set forth in SEQ ID NOs:3-33. For example, mouse and human dimeric oxidation-resistant ApoA1 variant polypeptides are considered to be orthologs of each other.

The term “fragment”, “analog”, and “derivative” when referring to the polypeptide of the present invention (e.g., SEQ ID NOs:3-33), can refer to a polypeptide that retains essentially at least one biological function or activity as the reference polypeptide. Thus, an analog includes a precursor protein that can be activated by cleavage of the precursor protein portion to produce an active mature polypeptide. The fragment, analog, or derivative of the polypeptide described herein (e.g., SEQ ID NOs:3-33) may be one having conservative or non-conservative amino acid substitution. The substituted amino acid residues may or may not be encoded by the genetic code, or the substitution may be such that one or more of the substituted amino acid residues includes a substituent group, is one in which the polypeptide is fused with a compound such as polyethylene glycol to increase the half-life of the polypeptide, or one in which additional amino acids are fused to the polypeptide such as a signal peptide or a sequence such as polyhistidine tag which is employed for the purification of the polypeptide or the precursor protein. Such fragments, analogs, or derivatives are deemed to be within the scope of the present invention.

The inventors have found that the oxidation-resistant ApoA1 variant dimers of the present invention essentially are resistant, at least in part, to myeloperoxidase enzyme mediated oxidation and may have improved biological activity as the ApoA1 wild-type or naturally occurring or synthetic monomeric or dimeric ApoA1 variant polypeptides in the presence of an oxidant. As used herein, an “oxidation-resistant amino acid” is an amino acid that when present in an ApoA1 variant protein, having the amino acid sequence of SEQ ID NO:3-33 is less likely to be oxidized in the presence of an oxidant, for example, myeloperoxidase (MPO), MPO generated oxidant, an oxidant generated by the MPO/H₂O₂/Cl⁻ system, HOCl/OCl⁻, MPO generated reactive nitrogen species, oxidants generated by the MPO/H₂O₂/NO₂⁻ system, NO₂⁻, ONOO⁻, ONOOCO₂⁻, an oxidant created by the action of ONOO⁻ in the presence of CO₂ or HCO₃⁻ in a buffer, an oxidant created by a glutathione synthase deficiency, a monoaminooxidase deficiency, an oxidant generated by NADPH oxidase, hydrogen peroxide, hypochlorite ion, hypochlorous acid, singlet oxygen, ozone, NO, hydroxyl radical, superoxide anion, peroxyl radical, peroxides of alkali and alkaline earth metals, organic peroxy compounds, peroxy acids, peroxynitriles and mixtures thereof to an extent that would reduce the capability of the ApoA1 variant protein to efflux cholesterol from a lipid containing cell, such as, RAW264.7 murine macrophage cells by more than 1%, or more than 5%, or more than 10%, or more than 15%, or more than 20% when compared to the same protein not being in the presence of the oxidant. In some embodiments, an “oxidation-resistant amino acid” can include: leucine, alanine, valine, isoleucine, and phenylalanine. In some embodiments, the oxidation-resistant amino acid is phenylalanine.

### Dimeric Oxidation-Resistant ApoA1 Variant Polypeptides

In various embodiments, the invention provides dimers comprising two oxidation-resistant ApoA1 variant polypeptide monomers that have amino acid substitutions that confer protection against oxidation from various sources, including oxidants present or created during inflammatory reactions, for example, by macrophages and polymorphonuclear neutrophils (PMNs). As used herein, the term “polypeptide” or “polypeptide monomer” refers to a polymer of amino acids without regard to the length of the polymer; thus peptides, oligopeptides and proteins are included in this term. The term: “dimeric oxidation-resistant ApoA1 variant polypeptide” and “oxidation-resistant ApoA1 variant dimer” are synonymous and both relate to a dimer of two oxidation-resistant ApoA1 variant polypeptide subunit monomers.

The term “polypeptide” includes polypeptides produced using L- or D-amino acids (the two possible stereoisomers of an amino acid), such as, for example, α-helical peptides or polypeptides. While the oxidation-resistant ApoA1 variant dimers of the present invention can be manufactured in several ways, in some embodiments, the dimers are formed by covalent attachment of two oxidation-resistant ApoA1 variant polypeptide monomers. The oxidation-resistant ApoA1 variant polypeptide monomers can be purified or isolated from a mixture or they can be synthesized. As used herein, the term “isolated” means a polypeptide that has been cloned, synthesized, prepared in a biochemical, bacterial or cellular expression system and purified by removing unwanted extraneous materials, or purified from its natural environment. In some embodiments, an oxidation-resistant ApoA1 variant dimer of the present invention is a construct comprising two oxidation-resistant ApoA1 variant polypeptide monomers linked by a covalent bond, for example, an intermolecular disulfide bond between cysteine residues, wherein at least one oxidation-resistant ApoA1 variant polypeptide monomers of the dimer, has at least one tryptophan amino acid substituted with an oxidation resistant amino acid, for example, phenylalanine. In some embodiments, an oxidation-resistant ApoA1 variant dimer is a construct comprising two oxidation-resistant ApoA1 variant polypeptide monomers covalently attached by a linker, such as a polypeptide linker comprising 2-50 amino acids in length. In other embodiments, the oxidation-resistant ApoA1 variant dimers are typically essentially free of the corresponding monomer, e.g., greater than 90% free by weight, greater than 95% free by weight and preferably greater than 99% free by weight. In some embodiments, the oxidation-resistant ApoA1 variant polypeptides in the dimer can be the same or they can be different. In some embodiments, the oxidation-resistant ApoA1 variant polypeptide monomers can be covalently linked, for example, by a disulfide bond or a short peptide linker.

In some embodiments, an exemplary oxidation-resistant ApoA1 variant dimer of the present invention comprises a dimer of two oxidation-resistant ApoA1 variant polypeptide monomers, each monomer having an amino acid sequence as provided in any one of SEQ ID NOs: 3 or 4 (respectively, based on the amino acid sequences of human ApoA1-Milano and human ApoA1-Paris) wherein each monomer of the dimer has from one to four tryptophan amino acid residues substituted with phenylalanine amino acid residues. In some embodiments, the monomers are dimerized by disulfide bridging between two cysteine residues, at least one cysteine being present in each monomer.

In one embodiment, the oxidation-resistant ApoA1 variant dimer comprises two oxidation-resistant ApoA1 variant polypeptide monomers, wherein each monomer has the same amino acid sequence, and wherein four tryptophan residues of the naturally occurring ApoA1-Milano polypeptide of SEQ ID NO:1 are substituted with phenylalanine residues as shown in SEQ ID NO:5. As used herein, the term “oxidation-resistant” at least when referring to dimeric ApoA1 variant polypeptides of the present invention, refers to a variant ApoA1 polypeptide, an ApoA1 polypeptide substitute or an ApoA1 variant polypeptide mimetic that retains biological activity in the presence of an oxidizing agent, for example, myeloperoxidase mediated oxidation, HOCl or other oxygen, nitrogen or chloride containing oxidants or free radicals.

In some embodiments, the inventor has targeted specific amino substitutions in the sequence of two exemplary variant ApoA1 polypeptides (Milano and Paris). In some embodiments, ApoA1 variant polypeptides include ApoA1-Milano (as exemplified by SEQ ID NO: 1); and ApoA1-Paris (as exemplified by SEQ ID NO:2). Included in the group of oxidation-resistant ApoA1 variant polypeptide monomers for the dimer constructs of the present invention include mutated ApoA1-Milano, ApoA1-Paris, functional fragments or orthologs thereof, wherein at least one of these oxidation-resistant ApoA1 variant polypeptide monomers in the dimer have a substitution of tryptophan with phenylalanine. In some embodiments, dimeric forms of ApoA1 variant polypeptides from other mammalian species such as mouse, rat, primate, monkey, hamster, dog, cat, pig and horse are included in the definition of ApoA1 variant polypeptides. In some embodiments, the ApoA1 variant polypeptide is ApoA1-Milano (SEQ ID NO:1) described in (Weisgraber et al., J. Biol. Chem. (1983) 258:2508-2513). Human ApoA1-Milano is naturally synthesized with a cysteine substituting an arginine at position 197 of the precursor protein or at position 173 of the mature form as shown in SEQ ID NO:1. In some embodiments, the ApoA1 variant polypeptide is ApoA1-Paris (SEQ ID NO:2) described in (Bielicki and Oda, Biochemistry (2002) 41:2089-2096). Human ApoA1-Paris contains a cysteine substituting an arginine at position 175 of the precursor protein or at position 151 of the mature form as shown in SEQ ID NO:2.

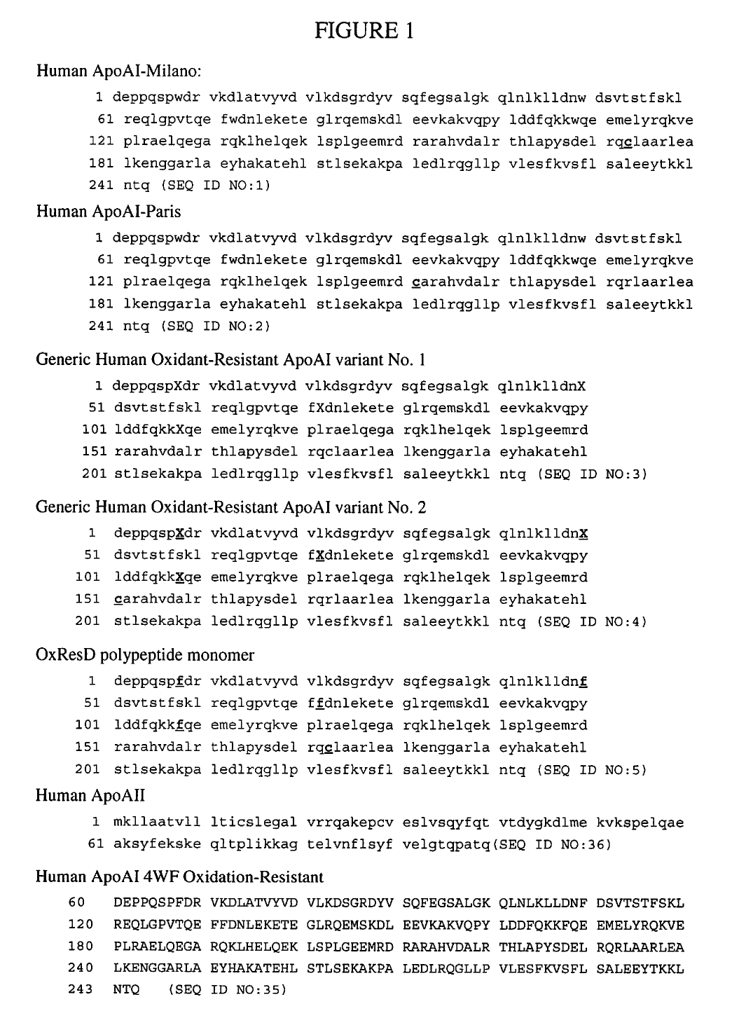

The oxidation-resistant ApoA1 variant dimers of the present invention include dimers comprising an ApoA1 variant polypeptide monomer that has an amino acid sequence wherein from one to four tryptophan amino acids are substituted with an oxidation-resistant amino acid. As used herein, “oxidation-resistant amino acids” can include: phenylalanine, leucine, alanine, valine and isoleucine. Each oxidation-resistant ApoA1 variant dimer has a pair of oxidation-resistant ApoA1 variant polypeptide monomers, wherein at least one monomer of the dimer is independently selected from the group of SEQ ID NOs:3-33, and wherein at least one monomer of the pair contains at least one tryptophan amino acid substitution with an oxidation resistant amino acid. In one embodiment, the oxidation resistant amino acid is phenylalanine. Representative oxidation-resistant polypeptide monomers in accordance with this example can include the following amino acid sequences: (SEQ ID NOs:3-4) wherein at least one X amino acid residue is an oxidation resistant amino acid, for example, phenylalanine. As shown in FIG. 1, SEQ ID NOs: 3 & 4, X indicates tryptophan or an oxidation-resistant amino acid. In some embodiments, X is tryptophan or phenylalanine. In some embodiments, at least one X amino acid is an oxidation-resistant amino acid, preferably phenylalanine. In other embodiments, the oxidation-resistant ApoA1 variant monomers contain four tryptophan amino acid residues substituted with four phenylalanine residues. In some embodiments, the oxidation-resistant ApoA1 variant dimer comprises two oxidation-resistant ApoA1 variant polypeptide monomers, each monomer having the amino acid sequence of SEQ ID NO:5 as shown in FIG. 1. In this embodiment, both oxidation-resistant ApoA1 variant polypeptide monomers comprise the amino acid sequence of SEQ ID NO:5 and are bound together via an intermolecular disulfide bridge formed at cysteine 173 in each monomer (highlighted as C in SEQ ID NO:5, shown in FIG. 1).

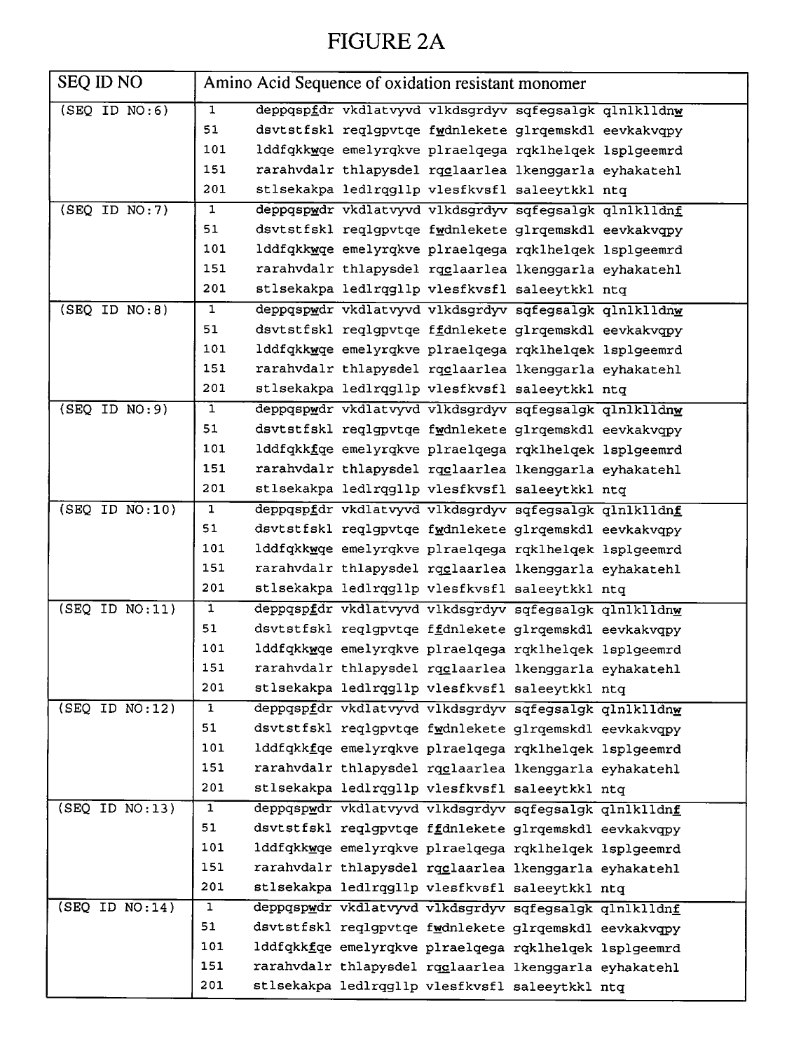

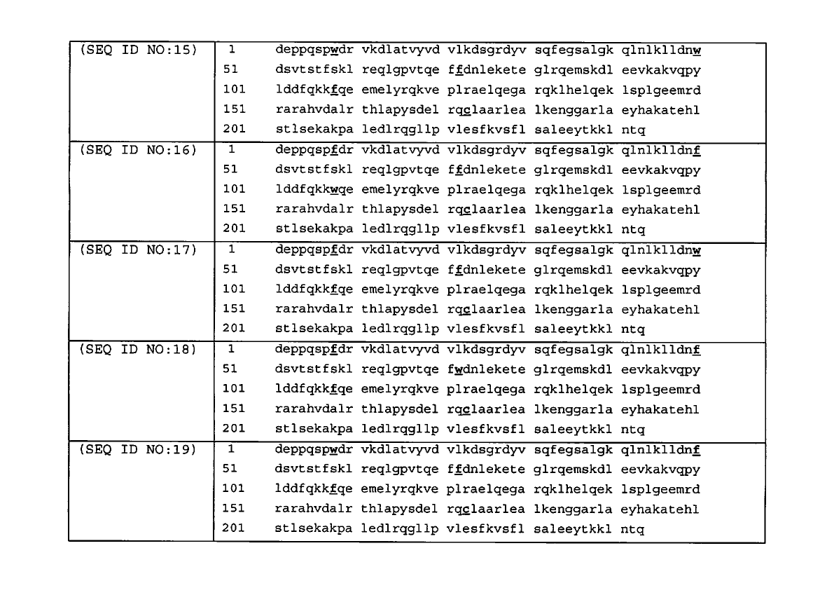

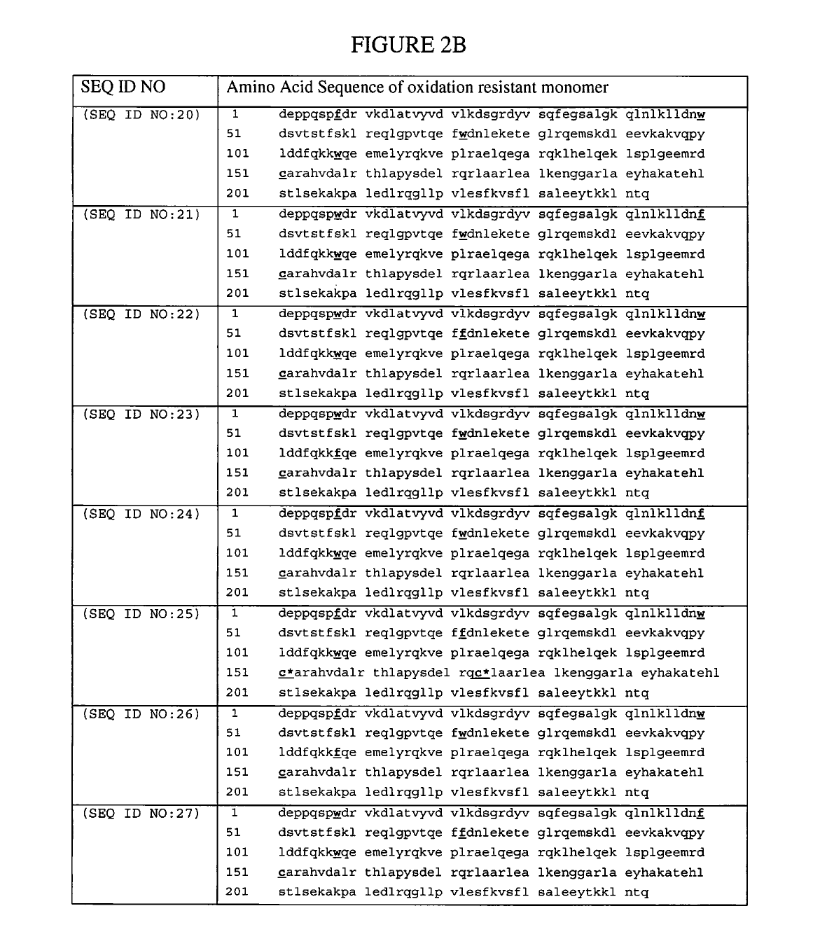

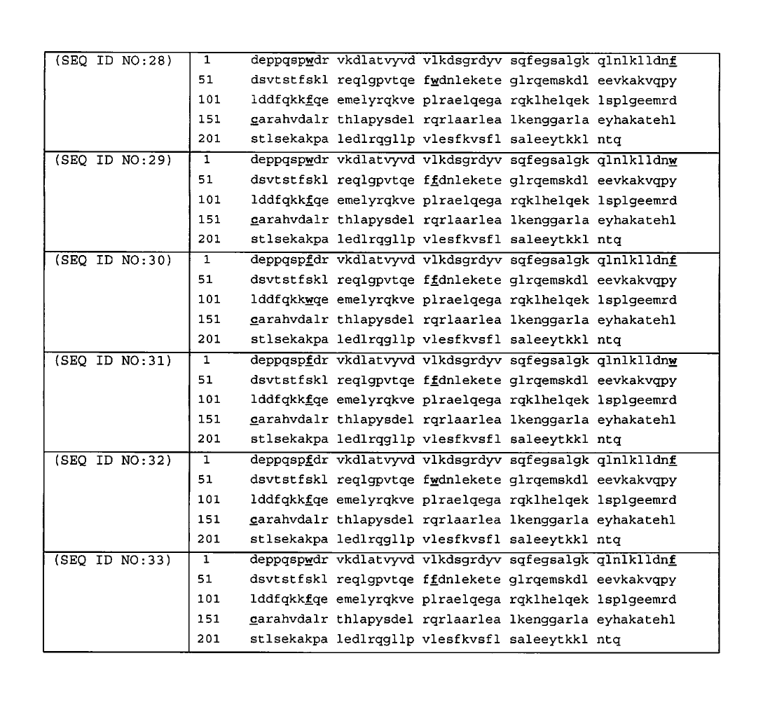

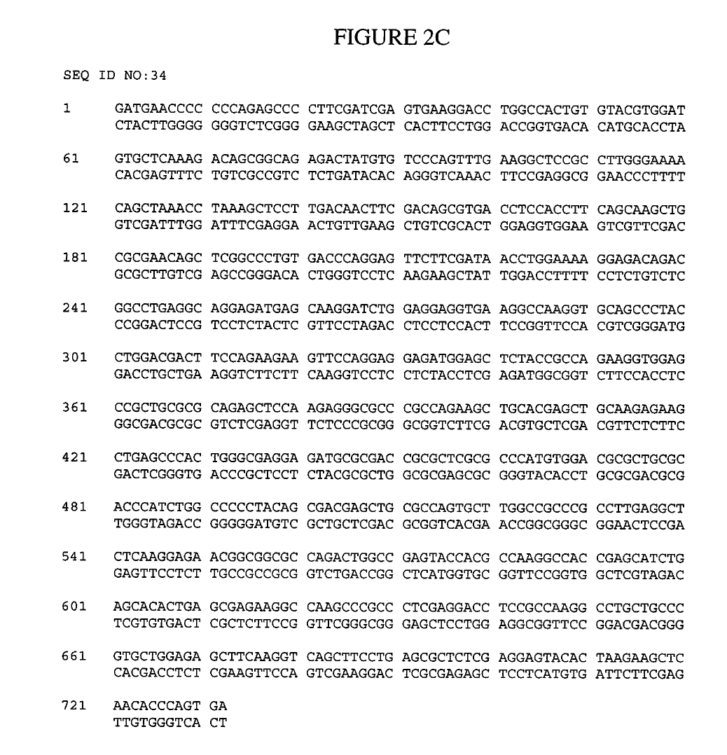

In some embodiments, the oxidation-resistant ApoA1 variant dimers can be constructed using any two oxidation-resistant ApoA1 variant polypeptide monomers having an amino acid sequence (SEQ ID NOs:5-33) as shown in FIGS. 1, 2A, and 2B. Each of the exemplified oxidation-resistant ApoA1 variant polypeptide monomers in FIGS. 2A, and 2B are based on the amino acid sequences provided in SEQ ID NOs:3-4 respectively, wherein at least one tryptophan amino acid is substituted with a phenylalanine.

In some embodiments, the oxidation-resistant ApoA1 variant dimers of the present invention can include a homodimer comprising two oxidation-resistant ApoA1 variant polypeptide monomers, for example, a first and a second monomer, wherein each monomer has the same amino acid sequence selected from SEQ ID NO:3-33. As used herein, a heterodimer can include a first monomer and a second monomer. In some embodiments, the first monomer of the heterodimer can comprise a monomer polypeptide having an amino acid sequence of SEQ ID NO:3-33. The second monomer of the heterodimer can be any polypeptide operable to form a linkage with the first monomer. In illustrative examples, the linkage can be a covalent linkage, a disulfide linkage, a linkage with a peptide linker or some other spacer or linker molecule known to those in the art. The heterodimer is also capable of reverse cholesterol efflux in vitro as measured using the method in Examples 3 and 4 herein, wherein the heterodimer has at least 20% cholesterol efflux activity of the wild-type human ApoA1 tested under the conditions of the in vitro assay in Example 4. In some embodiments, the second monomer or first monomer can be a cysteine containing protein. In some embodiments, one of the monomers of the heterodimer can include the amino acid sequence of human ApoA1, human ApoAII or human ApoA4, (for example, human ApoA4, NCBI Accession No. NM_{—}00482.3). Alternatively, in some embodiments, the oxidation-resistant ApoA1 variant dimers of the present invention can include a heterodimer, comprising two oxidation-resistant ApoA1 variant polypeptide monomers, for example, a first monomer and a second monomer, wherein each of the first and second monomers has a different amino acid sequence, each of the first and the second monomer amino acid sequence selected from SEQ ID NO:3-33. In still other embodiments, the oxidation-resistant ApoA1 variant dimers of the present invention can include a heterodimer, comprising two monomers, wherein the first monomer has an amino acid sequence selected from SEQ ID NO:3-33, and the second monomer comprises human ApoA1 (GenBank Accession No. CAA01253.1), or human ApoA2 (NCBI Reference Sequence NP_{—}001634.1 (or the mature form thereof), either one of the second monomers of human ApoA1 or ApoA2, having none or at least one oxidation resistant amino acid substitution as presently described herein.

In some embodiments, the oxidation-resistant ApoA1 variant dimers of the present invention can include a heterodimer, wherein the heterodimer includes a first monomer having an amino acid sequence selected from SEQ ID NO:3-33, and a second monomer comprising human ApoA1 (GenBank Accession No. CAA01253.1), or human ApoA2 (NCBI Reference Sequence NP_{—}001634.1 or the mature form thereof). In some embodiments, the second monomer can comprise human ApoA1 or ApoA2. In some embodiments, the second monomer can further have none or at least one oxidation resistant amino acid substitution as presently described herein. In another embodiment, the second monomer can optionally also have an amino acid substitution wherein at least one arginine in the amino acid sequence of the second monomer is substituted with a cysteine. In some embodiments, these two forms of amino acid substitutions may be present or only one of these substitutions may be present or none of the substitutions may be present in the second monomer.

When the first and second monomers of the heterodimer cannot form a disulfide bridge, the first and second monomers can be linked together by covalent attachment of the first and second monomers with a linker, for example, a small peptide linker. The linker can be attached to the first and second monomers at the N-terminal portion of each monomer, the C-terminal portion of each monomer, or any amino acid along the sequence of each monomer. Other methods for linking two polypeptides are well known in the art. Chemical cross-linking the two monomers of a heterodimer containing one cysteine can involve the use of a cross-linking reagent (also known as “cross-linker”), typically a bifunctional (two-armed) chemical linker that forms covalent linkages between two or more peptides, can be used to covalently link peptides to each other. Such bifunctional linkers can be homobifunctional (wherein both “arms” of the linker are the same chemical moiety) or heterobifunctional (wherein each of the two “arms” is a different chemical moiety than the other).

Hermanson (Bioconjugate Techniques, Academic Press, (1996)), herein incorporated by reference, summarizes many of the chemical methods used to link proteins and other molecules together using various reactive functional groups present on various cross-linking or derivatizing reagents. Cross-linking agents are based on reactive functional groups that modify and couple to amino acid side chains of proteins and peptides, as well as to other macromolecules. Cross-linking reagents incorporate two or more functional reactive groups. The functional reactive groups in a cross-linking reagent may be the same or different. Many different cross-linkers are available to cross-link various proteins, peptides, and macromolecules. The chemical modification may be done using cross-linking reagents containing selective groups that react with primary amines, sulfhydryl (thiol) groups, carbonyl, carboxyl groups, hydroxyl, or carbohydrates and other groups placed on a polypeptide, especially by posttranslational modifications within the cell. The selective groups include, but are not limited to, imidoester, N-hydroxysuccinimide ester or sulfosuccinimidyl ester, ester of 1-hydroxy-2-nitrobenzene-4-sulfonic, maleimide, pyridyl disulfide, carbodiimide, and haloacetyl groups.

Sulfhydryl reactive functional groups include maleimides, alkyl and aryl halides, haloacyls, haloacetyls and pyridyl disulfides. Maleimides, alkyl and aryl halides, haloacetyls and haloacyls react with thiols to form stable thioether bonds that are not reduced by reagents such as 2-mercaptoethanol and dithiothreitol. Pyridyl disulfides form mixed disulfides with thiol groups, mixed disulfides may be used as an intermediate for cross-linking two or more macromolecules. Cross-linkers that first react with a carboxyl group to form an activated intermediate and then reacts with an amino group, such as an amino group of lysine or an amino group of an amino terminal amino acid, may be used.

A spacer arm or “cross-bridge” region, consisting of a spacer group or a functional group, such as a disulfide bond or hindered disulfide bond, connects the two selective or functional groups. The length of the spacer arm may be varied. The distance between the functional groups establishes the length of the spacer arm. Longer spacer arms may be required to diminish or eliminate steric hindrance between two molecules that are cross-linked together. Intermolecular cross-linking is more efficient with longer spacer arms. Spacer arms may have reactive bonds within them that enable further modifications. For example, internal cleavable bonds may be placed within the spacer, such as disulfides or hindered disulfides, one or more ester bonds, or vicinal hydroxyl groups. Cleavage of internal disulfide bonds may be achieved using reduction with thiol containing reagents such as 2-mercaptoethanol and dithiothreitol. One or more metabolizable bonds may be inserted internally in the cross-linking reagent to provide the ability for the coupled entities to separate after the bond(s) is broken after the conjugate is transported into the cell and into the body. Homobifunctional cross-linkers contain at least two identical functional groups. Heterobifunctional cross-linkers contain two or more functional reactive groups that react with different specificity. Because heterobifunctional cross-linkers contain different reactive groups, each end can be individually directed towards different functional groups on proteins, peptides, and macromolecules. This feature results in linking, for example, amino groups on one molecular entity to carboxyl groups on another entity, or amino groups on one entity to sulfhydryl groups on another entity.

In some embodiments, the isolated oxidation-resistant ApoA1 variant dimer comprises an oxidation-resistant ApoA1 variant dimer wherein the first and the second monomers are the same. In some embodiments, the isolated oxidation-resistant ApoA1 variant dimer has two monomers, each monomer comprising an amino acid sequence of SEQ ID NO:5. In other embodiments, the isolated oxidation-resistant ApoA1 variant dimer can comprise two monomers, wherein one of the two monomers has an amino acid sequence of SEQ ID NO:6-33. Alternatively, the isolated oxidation-resistant ApoA1 variant dimer of the present invention can include a dimer in which the first and said second monomers comprise an amino acid sequence of SEQ ID NO:6-33.

In some embodiments, the isolated oxidation-resistant ApoA1 variant dimer can include a first monomer comprising an amino acid sequence of SEQ ID NOs:6-33 and a second monomer comprising an amino acid sequence of SEQ ID NO:35 or 36. In this particular embodiment, the second monomer can optionally include at least one amino acid substitution of a tryptophan residue for an oxidation resistant amino acid, for example, phenylalanine. Alternatively or in addition to the at least one amino acid substitution of a tryptophan residue for an oxidation resistant amino acid, the second monomer can optionally comprise a substitution of an arginine residue for a cysteine residue.

In some embodiments, the first and second monomers may not be linked with a disulfide bridge, but rather are linked a peptide linker.

In some embodiments, the isolated oxidation-resistant ApoA1 variant dimer can comprise a first monomer and a second monomer, wherein at least one of the first and second monomers comprises an amino acid sequence having at least 70%, at least 75%, at least 80%, at least 85%, at least 90%, at least 95%, at least 97%, at least 98% or at least 99% sequence identity to SEQ ID NOs:3-33, or a functional fragment or variant thereof. The oxidation-resistant ApoA1 variant dimers of the present invention can also include a dimeric construct comprising two oxidation-resistant ApoA1 variant polypeptide monomers, each monomer having at least 70%, at least 80%, at least 85%, at least 90%, at least 95%, at least 97%, at least 98% or at least 99% sequence identity to any one of SEQ ID NOs:3-33, or a fragment thereof. In some embodiments, the isolated oxidation-resistant ApoA1 variant dimer can comprise a first and second monomer, wherein the amino acid sequence of at least one of the first and the second monomer is substituted with conservative amino acid residues with the exception of phenylalanine substitution.

The conservatively substituted polypeptides used to form the oxidation-resistant ApoA1 variant dimer do not have a reduced ability to efflux cholesterol from a lipid laden cell using the methods provided in the examples section below, and in the presence of an oxidant as exemplified herein, when compared to a dimer of SEQ ID NO:5 by more than 20%. In some embodiments, illustrative oxidation-resistant ApoA1 variant dimers of the present invention include a dimeric construct comprising two oxidation-resistant ApoA1 variant polypeptide monomers, wherein at least one of the two monomers has at least 70%, at least 80%, at least 85%, at least 90%, at least 95%, at least 97%, at least 98% or at least 99% sequence identity to any one of SEQ ID NOs:3-33. In a preferred embodiment, an illustrative oxidation-resistant ApoA1 variant dimers of the present invention include a dimeric construct comprising two oxidation-resistant ApoA1 variant polypeptide monomers, each monomer having at least 70%, at least 80%, at least 85%, at least 90%, at least 95 percent, at least 97%, at least 98% or at least 99% sequence identity to any one of SEQ ID NO:5.

In some embodiments, functional fragments and orthologs of the oxidation-resistant ApoA1 variant dimers exemplified above, can include oxidation-resistant ApoA1 variant dimers comprising two oxidation-resistant ApoA1 variant polypeptide monomers, each monomer comprising an amino acid sequence as in SEQ ID NO:5-33, wherein, 1-30% of the amino acid sequence of each oxidation-resistant ApoA1 variant polypeptide monomer of the dimer can be substituted with a conservative amino acid substitution, with the exception of substitution of an oxidation-resistant amino acid, such that the functional fragment or ortholog dimer still retains biological activity when compared to the oxidation-resistant ApoA1 variant dimers of the present invention. Such biological activity can include cholesterol efflux, reduction in inflammatory cellular, chemokine or cytokine activity. Any substitution mutation is conservative in that it minimally disrupts the biochemical and/or biological properties of the oxidation-resistant ApoA1 variant dimer.

### Conjugated Forms of Oxidation-Resistant ApoA1 Variant Dimers

In some embodiments, the present invention also broadly includes chemically modified oxidation-resistant ApoA1 variant dimers. In one example, oxidation-resistant ApoA1 variant dimers can also be linked or conjugated with agents that provide desirable biochemical, pharmaceutical or pharmacodynamic properties. As used herein, the term “conjugated oxidation-resistant ApoA1 variant dimer” refers to an oxidation-resistant ApoA1 variant dimer having at least one monomer of that dimer covalently attached to a molecule, for example, polyethylene glycol, that provides a desirable pharmaceutical or pharmacodynamic property, or a molecule that permits improved purification of the oxidation-resistant ApoA1 variant polypeptide monomers and/or dimers. The oxidation-resistant ApoA1 variant polypeptide monomers can be stably linked to a molecule, for example, a polymer such as polyethylene glycol and pegylated to specific amino acid residues in the oxidation-resistant ApoA1 variant polypeptide monomer to obtain desirable properties of solubility, stability, half-life and other pharmaceutically advantageous properties. (See for example Davis et al. Enzyme Eng. (1978) 4:169-73; Burnham, Am. J. Hosp. Pharm. (1994) 51:210-218, which are incorporated by reference).

In general, the chemical modification contemplated is an oxidation-resistant ApoA1 variant dimer construct wherein at least one oxidation-resistant ApoA1 variant polypeptide monomer is linked to at least one polymer molecule, for example, polyethylene glycol molecule to form a pegylated oxidation-resistant ApoA1 variant dimer. Pegylation of oxidation-resistant ApoA1 variant polypeptide monomers or dimers may be carried out by any of the pegylation reactions known in the art. See, for example: Focus on Growth Factors (1992) 3(2):4-10; EP 0 154 316; EP 0 401 384; and the other publications cited herein that relate to pegylation. Examples are made in reference to pegylation of both polypeptide monomers that are subsequently covalently attached or dimers, if such pegylation permits dimeric conformation. In some embodiments, the pegylation can be carried out via an acylation reaction or an alkylation reaction with a reactive polyethylene glycol molecule (or an analogous reactive water-soluble polymer).

In some embodiments, polyethylene glycol is an example of a polymer molecule that can be covalently attached to an oxidation-resistant ApoA1 variant polypeptide monomer or dimer thereof. The polymer molecules used in both the acylation and alkylation approaches may be selected from among water soluble polymers or a mixture thereof. The polymer selected should be water soluble so that the protein to which it is attached does not precipitate in an aqueous environment, such as a physiological environment. The polymer selected should be modified to have a single reactive group, such as an active ester for acylation or an aldehyde for alkylation, preferably, so that the degree of polymerization may be controlled as provided for in the present methods. A preferred reactive PEG aldehyde is polyethylene glycol propionaldehyde, which is water stable, or mono C₁₋₁₀ alkoxy or aryloxy derivatives thereof (see, U.S. Pat. No. 5,252,714). The polymer may be branched or unbranched. Preferably, for therapeutic use of the end-product preparation, the polymer will be pharmaceutically acceptable. The water soluble polymer may be selected from the group consisting of, for example, polyethylene glycol, monomethoxy-polyethylene glycol, dextran, poly-(N-vinyl pyrrolidone) polyethylene glycol, propylene glycol homopolymers, a polypropylene oxide/ethylene oxide co-polymer, polyoxyethylated polyols (e.g., glycerol) and polyvinyl alcohol. For the acylation reactions, the polymer(s) selected should have a single reactive ester group. For the present reductive alkylation, the polymer(s) selected should have a single reactive aldehyde group. Generally, the water soluble polymer will not be selected from naturally-occurring glycosyl residues since these are usually made more conveniently by mammalian recombinant expression systems. The polymer may be of any molecular weight, and may be branched or unbranched.

Another consideration for selecting a conjugated polymer is the molecular weight of the polymer. In general, the higher the molecular weight of the polymer, the fewer number of polymer molecules which may be attached to the protein. Similarly, branching of the polymer should be taken into account when optimizing these parameters. Generally, the higher the molecular weight (or the more branches) the higher the polymer:protein ratio. In general, for the pegylation reactions contemplated herein, the preferred average molecular weight is about 2 kDa to about 100 kDa (the term “about” indicating ±1 kDa). The preferred average molecular weight of the PEG polymer can be from about 5 kDa to about 50 kDa, particularly preferably about 12 kDa to about 25 kDa, and most preferably 20 kDa. The ratio of water-soluble polymer to oxidation-resistant ApoA1 variant dimer will generally range from 1:1 to 100:1, preferably (for polypegylation) 1:1 to 20:1 and (for monopegylation) 1:1 to 5:1.

In some embodiments, pegylation by acylation generally involves reacting an active ester derivative of polyethylene glycol (PEG) with an oxidation-resistant ApoA1 variant polypeptide monomer or dimer. Any known or subsequently discovered reactive PEG molecule may be used to carry out the pegylation of an oxidation-resistant ApoA1 variant polypeptide monomer or dimer. A preferred activated PEG ester is PEG esterified to N-hydroxysuccinimide (“NHS”). As used herein, “acylation” is contemplated to include without limitation the following types of linkages between oxidation-resistant ApoA1 variant polypeptide monomer or dimer and a water soluble polymer such as PEG: amide, carbamate, urethane, and the like. See Bioconjugate Chem. (1994) 5:133-140. Reaction conditions can be selected from any of those known in the pegylation art or those subsequently developed, but should avoid conditions such as temperature, solvent, and pH that would inactivate the oxidation-resistant ApoA1 variant dimer species to be modified.

Pegylation by acylation will generally result in a poly-pegylated oxidation-resistant variant polypeptide, wherein the lysine ε-amino groups are pegylated via an acyl linking group. Preferably, the connecting linkage will be an amide. Also preferably, the resulting product will be substantially only (e.g., >95%) mono, di- or tri-pegylated. However, some species with higher degrees of pegylation (up to the maximum number of lysine ε-amino acid groups of an oxidation-resistant ApoA1 variant polypeptide monomer or dimer plus one α-amino group at the amino termini of an oxidation-resistant ApoA1 variant polypeptide monomers or dimer will normally be formed in amounts depending on the specific reaction conditions used. If desired, more purified pegylated species may be separated from the mixture, particularly unreacted species, by standard purification techniques, including, among others, dialysis, salting-out, ultrafiltration, ion-exchange chromatography, gel filtration chromatography and electrophoresis.

In other embodiments useful in the synthesis of pegylated oxidation-resistant ApoA1 variant polypeptide monomers or dimers, pegylation by alkylation generally involves reacting a terminal aldehyde derivative of PEG with an oxidation-resistant ApoA1 variant polypeptide monomer or dimer in the presence of a reducing agent. Pegylation by alkylation can also result in poly-pegylated oxidation-resistant ApoA1 variant polypeptide monomers or dimer. In addition, one can manipulate the reaction conditions as described herein to favor pegylation substantially only at the α-amino group of the N-terminus of the oxidation-resistant ApoA1 variant monomer or dimer species (i.e., a mono-pegylated species). In either case of monopegylation or polypegylation, the PEG groups are preferably attached to the polypeptide via a —CH₂—NH— group.

Derivatization via reductive alkylation to produce a monopegylated product exploits differential reactivity of different types of primary amino groups available for derivatization of an oxidation-resistant ApoA1 variant polypeptide monomers or dimer. The reaction can be performed at a pH which allows use of the pKₐ differences between the ε-amino groups of the lysine residues and that of the α-amino group of the N-terminal residues of the polypeptides in the dimer. By such selective derivatization, attachment of a water soluble polymer that contains a reactive group such as an aldehyde, to a protein is controlled: the conjugation with the polymer takes place predominantly at the N-terminus of the polypeptide and no significant modification of other reactive groups, such as the side chain amino groups, for example, those found in lysine.

In an illustrative embodiment, the present invention includes pegylated oxidation-resistant ApoA1 variant dimers, wherein the PEG group(s) is (are) attached via acyl or alkyl groups. As discussed above, such products may be mono-pegylated or poly-pegylated (e.g., containing 2-6, preferably 2-5, PEG groups). The PEG groups can be generally attached to the protein at the α or ε amino groups of amino acids, but it is also contemplated that the PEG groups could be attached to any amino group attached to the polypeptide, which is sufficiently reactive to become attached to a PEG group under suitable reaction conditions.

In some embodiments, the conjugated form of the oxidation-resistant ApoA1 variant dimer includes a composition comprising oxidation-resistant ApoA1 variant dimer conjugated to PEG or some other polymer molecule. The conjugated dimer preparation will preferably be greater than 90% conjugated oxidation-resistant ApoA1 variant dimer, and more preferably greater than 95% conjugated oxidation-resistant ApoA1 variant dimer when compared to non-conjugated oxidation-resistant ApoA1 variant dimers. The conjugated oxidation-resistant ApoA1 variant dimer has ApoA1 biological activity, i.e. retains the ability to directly or indirectly cause promotion of cholesterol efflux, (for example, to the extent of at least about 50% activity of non-conjugated oxidation-resistant ApoA1 variant dimers of the present invention, or at least about 60%, or at least about 70%, or at least about 80% or at least about 90% of the activity of the non-conjugated oxidation-resistant ApoA1 variant dimers of the present invention, or reduce the negative effects of inflammatory mediators, even in the presence of an oxidizing agent, for example, myeloperoxidase, or NADPH-oxidase mediated oxidation, HOCl or other oxygen, nitrogen or chloride oxidants or free radicals.

In other embodiments, the oxidation-resistant ApoA1 variant polypeptide monomers used in the making of the dimers of the present invention can also include a protein or non-protein tag or marker. Detection of the oxidation-resistant ApoA1 variant polypeptide monomers during the purification of the recombinantly expressed oxidation-resistant ApoA1 variant polypeptide monomers or for use during the biological assays to determine the activity and/or location of the oxidation-resistant ApoA1 variant dimers in vitro/in vivo by immunoassays, such as immunohistocytochemistry, may incorporate oxidation-resistant ApoA1 variant polypeptide monomers that are labeled or tagged. Epitope tagging is a technique widely employed for detecting proteins and peptides, and may be particularly useful for discriminating among similar proteins or peptides that are not distinguishable with conventional antibodies. Epitope tagging involves adding a unique epitope tag peptide sequence to the protein of interest by recombinant DNA techniques, thereby creating a fusion protein. The resulting tagged protein can then be detected with an antibody specific for the epitope tag.

Epitope tagging of a protein employs conventional methodologies known to the skilled artisan. This process can involve two DNA molecules: (1) a polynucleotide which is cloned in a plasmid vector and which includes a sequence of nucleotides encoding the protein as well as regulatory sequences (i.e. promoter, translations start, etc.) needed to express the protein; and (2) an oligonucleotide encoding the epitope with which the polypeptide is to be tagged. The oligonucleotide is designed to encode, in one of its reading frames, an epitope recognized by a known antibody or other purification media. A site in the polynucleotide protein coding sequence for insertion of the oligonucleotide is designated. This site may be located at or near the 3′ or the 5′ end of the coding sequence of the polypeptide, or somewhere in between the 3′ and 5′ ends. The insertion site for the oligonucleotide is typically a unique restriction site. The plasmid is then linearized with the restriction endonuclease, and the oligonucleotide is ligated into the site.

The small size of the epitope tag, which is usually 2-50, or preferably from 5-20 amino acids in length, generally has no effect on the biological function of the tagged protein. This contrasts with many larger fusion proteins, in which the activity or function of the fusion protein is affected by longer peptide label. Using conventional epitope tagging techniques, as is known to the skilled artisan, hundreds of different proteins have been epitope-tagged with numerous distinct peptides. These include: the c-myc epitope (Evans et al., (1985)); hexahistidine (His₆ₓ) epitope (Caspers et al., (1991)); Glutathione glutathione S-transferase (GST) epitope (Sato et al., (1983)); the HA-epitope (Niman et al., (1983)) the FLAG epitope (Hopp et al., (1988)); the epsilon-tag epitope (Olah et al., (1994)); the AU1 and AU5 epitopes (Lim et al., (1990)); the glu-glu epitope (Grussenmeyer et al., (1985)); the KT3 epitope (MacArthur et al., (1984)); the IRS epitope (Liang et al., (1996)); the BTag epitope (Wang et al., (1996)); and the vesicular stomatitis virus (VSV) epitope (Kreis et al., (1986)). Essentially any peptide can be used as an immunogen to raise antibodies that will recognize that same peptide when it is present within or at the termini of a polypeptide or protein.

In some embodiments, epitope tagging of the present oxidation-resistant ApoA1 variant dimers may be advantageous in the use of antibodies with known characteristics for purposes of facilitating purification and identification in tissues and in vitro methods, and makes extensive characterization of new antibodies unnecessary. Epitope tagging is far less time consuming than the traditional method of producing an antibody to the specific protein being studied. In some embodiments, the oxidation-resistant ApoA1 variant dimers of the present invention comprise at least one oxidation-resistant ApoA1 variant polypeptide monomer that has been tagged with an epitope, for example, polyhistidine (His₆ₓ), which enables the convenient purification of the polypeptide monomers using a Ni²⁺ column, which will bind the polyhistidine sequence and thereby the whole polypeptide. After elution from the column, the polyhistidine sequence may be cleaved off by a proteinase such as thrombin recognizing a specific sequence built into the construct between the polypeptide construct and the polyhistidine sequence attached thereto.

In other embodiments, the oxidation-resistant ApoA1 variant dimers of the present invention can also further incorporate a radiolabel covalently attached to the primary structure of one of the dimer's polypeptide monomers. Various amino acids can be candidate locations for radiolabelling. Methods for radiolabelling proteins with ³⁵S, for example, [³⁵S]methionine and [³⁵S]cysteine are well known in the art. Other radiolabels can also be employed and are considered within the scope of the present invention. As an example, the method of Chen, L-C, and Casadevall, A, Anal. Biochem. (1999) 269(1):179-188 can be used to label the oxidation-resistant ApoA1 variant dimers, either as a dimer or as their constitutive polypeptides.

### Production of Oxidation-Resistant ApoA1 Variant Dimers

In some embodiments, the oxidation-resistant ApoA1 variant dimers of the present invention comprise two oxidation-resistant ApoA1 variant monomers that are dimerized through intermolecular disulfide bonding between the cysteine amino acid residue present in each of the oxidation-resistant ApoA1 variant polypeptide monomers. In the first step in making or isolating the inventive dimers, involves the synthesis of the oxidation-resistant ApoA1 variant polypeptide monomers. In an illustrative example at least one of the oxidation-resistant ApoA1 variant polypeptide monomers which makes up the oxidation-resistant ApoA1 variant dimer has an amino acid sequence as set forth in SEQ ID NOs: 3-33, as shown in FIGS. 1-2B. In some embodiments, the oxidation-resistant ApoA1 variant polypeptide monomers are synthesized, preferably using recombinant techniques. The oxidation-resistant ApoA1 variant polypeptide monomers can be produced recombinantly by expressing a nucleic acid in an expression vector, using standard and well-established techniques known in the field of molecular biology. In this regard, the practice of the present invention will employ, unless otherwise indicated, conventional techniques of molecular biology, microbiology, recombinant DNA technology and immunology that are within the skill of those working in the art. Such techniques are explained fully in the literature. Examples of particularly suitable texts for consultation include the following: Sambrook and Russell, Molecular Cloning; A Laboratory Manual, Second Edition (1989); DNA Cloning, Volumes I and II (D. N Glover ed. 1985); Oligonucleotide Synthesis (M. J. Gait ed. (1984)): Nucleic Acid Hybridization (B. D. Hames & S. J. Higgins eds. (1984)); Transcription and Translation (B. D. Hames & S. J. Higgins eds. (1984)); Animal Cell Culture (R. I. Freshney ed. (1986)); Immobilized Cells and Enzymes (IRL Press, (1986)); B. Perbal, A Practical Guide to Molecular Cloning (1984); the Methods in Enzymology series (Academic Press, Inc.), especially volumes 154 & 155; Gene Transfer Vectors for Mammalian Cells (J. H. Miller and M. P. Calos eds. (1987), Cold Spring Harbor Laboratory); Immunochemical Methods in Cell and Molecular Biology (Mayer and Walker, eds. (1987), Academic Press, London); Scopes, (1987) Protein Purification: Principles and Practice, Second Edition (Springer Verlag, N.Y.); and Handbook of Experimental Immunology, Volumes I-IV (D. M. Weir and C. C. Blackwell eds. (1986)) which are all incorporated by reference herein in their entireties.

As described above, specific oxidation-resistant ApoA1 variant polypeptide monomers can be derived by mutating the sequence of ApoA1 variant proteins of SEQ ID NO:1-2 by substituting at least one and up to four tryptophan residues with phenylalanine amino acid residues.

In some embodiments, the oxidation-resistant ApoA1 variant polypeptide monomers can be prepared by site-specific mutagenesis of nucleotides in the cDNA encoding ApoA1 (for example, the mutated sequence of SEQ ID NO:34 as shown in FIG. 2C), or may be prepared by in vitro synthesis using established techniques. For example, the oxidation-resistant ApoA1 variant polypeptide monomers can be synthesized using standard direct peptide synthesizing techniques (Bodanszky, Principles of Peptide Synthesis, Springer-Verlag, Heidelberg, Germany, 1984), such as solid-phase synthesis (see, e.g., Merrifield, J. Am. Chem. Soc. (1993) 85:2149-54.

The nucleic acid sequences encoding the oxidation-resistant ApoA1 variant polypeptide monomers of the present invention can be inserted and ligated into a suitable episomal or non-homologously integrating vectors, e.g., bacterial or viral vectors, which can be introduced in the appropriate host cells by any suitable means (transformation, transfection, conjugation, protoplast fusion, electroporation, calcium phosphate-precipitation, direct microinjection, etc.). In some embodiments, certain well-known factors can be considered when selecting a particular plasmid or viral vector including: the ease of selection of the transformed host cells as opposed to non-transformed host cells; the tropism of the vector towards a desired host cell; the number of copies of the vector which are desired in a particular host; and whether it is desirable to be able to “shuttle” the vector between host cells of different species.

In some embodiments, a polynucleotide encoding an oxidation-resistant ApoA1 variant polypeptide monomer, such as SEQ ID NO:34, be subcloned into an appropriate expression vector using well known molecular genetic techniques. Site-specific mutagenesis may be performed using cassette or PCR mutagenesis or other techniques well known in the art, for example, to produce a DNA construct, such as a DNA vector suitable for expression of the oxidation-resistant ApoA1 variant polypeptide monomers of the present invention in a suitable host cell. For example, an oxidation-resistant ApoA1 variant polypeptide monomer can be produced as a recombinant protein expressed in yeast or E. coli as described in U.S. Pat. No. 5,721,114 and European Patents EP 0 469 017 and EP 0 267 703, incorporated herein by reference in their entireties. Any appropriate expression vector (see, e.g., Pouwels, et al., Cloning Vectors: A Laboratory Manual, Elsevier, N.Y., (1985)) and corresponding suitable host cells can be employed for production of oxidation-resistant ApoA1 variant polypeptide monomers of the present invention. Expression hosts include, but are not limited to, bacterial species, mammalian or plant, and insect host cell systems including baculovirus systems (see, e.g., Luckow, et al., Bio/Technology (1988) 6:47.

The vector or vectors useful in the present invention, should allow the expression of the oxidation-resistant ApoA1 variant polypeptide monomers, functional fragments thereof or fusion proteins thereof containing a prepro or leader polypeptide, or a tag or affinity label in the prokaryotic or eukaryotic host cells under the control of transcriptional initiation/termination regulatory sequences. A “fusion” polypeptide can include an oxidation-resistant ApoA1 variant polypeptide monomer operatively linked to a heterologous polypeptide. A heterologous polypeptide can be linked at either the N-terminus, C-terminus or at some location between the N and C-termini of the oxidation-resistant ApoA1 variant polypeptide monomer. The regulatory sequences, for example, promoters, can be chosen to be constitutively active or inducible in the chosen host cell. A cell line substantially enriched in such cells can be then isolated to provide a stable cell line. Selection of specific transcriptional initiation/termination regulatory sequences can be made using routine considerations, for example, the eventual host cell in which the expression of the transgene will occur, and other known requirements of the specific vector being employed.

The oxidation-resistant ApoA1 variant polypeptide monomers can be produced by a suitable host cell such as a bacterium, a yeast, an insect, a mammalian, an avian and in higher plant cells. Host cells can be either prokaryotic or eukaryotic. Illustrative examples of eukaryotic hosts, include yeast cells, insect cells, plant cells, intact plants, cultivars and mammalian cells, such as 293, COS-7, C127, NIH-3T3, CHO, HeLa, BHK, SF9, SF21, Saccharomyces, Picchia, chicken hepatoma cells (LMH2A), safflower, rice, carrot, cells. These eukaryotic cells can be advantageous, because they provide post-translational modifications to proteins, including correct folding and glycosylation. A number of recombinant DNA strategies exist that utilize strong promoter sequences and/or high-copy number plasmids to produce the desired proteins in yeast. Yeast-based production of the oxidation-resistant ApoA1 variant polypeptide monomers can be advantageous because in most cases, yeast cells can recognize leader sequences in cloned mammalian gene products and secrete peptides bearing leader sequences (i.e., pre-peptides).

For eukaryotic hosts (e.g., yeasts, insect, plant, or mammalian cells), different transcriptional and translational regulatory sequences may be employed, depending on the nature of the host. They may be derived from viral sources, such as adenovirus, bovine papilloma virus, tobacco mosaic virus, Maloney Murine Leukemia virus, Simian virus or the like, where the regulatory signals are associated with a particular gene that has a high level of expression. Examples are the TK promoter of the Herpes virus, the SV40 early promoter, the yeast gal4 gene promoter, etc. Transcriptional initiation regulatory signals may be selected which allow for repression and activation, so that expression of the genes can be modulated. The cells that are stably transformed by the introduced DNA can be selected by introducing one or more markers for example, an antibiotic resistance gene, hygromycin B phosphotransferase gene, neomycin phosphotransferase gene, blasticidin deaminase and the like, allowing the selection of host cells which contain the expression vector. The marker may also provide for prototrophy to an auxotropic host biocide resistance, e.g., antibiotics, or heavy metals such as copper, or the like. The selectable marker gene can either be directly linked to the DNA gene sequences to be expressed, or introduced into the same cell by co-transfection.

In some embodiments, yeast cells can carry out post-translational peptide modifications including glycosylation and are particularly suitable for large-scale production of the desired oxidation-resistant ApoA1 variant polypeptide monomers. In some embodiments, yeast expression plasmids can be constructed using high-copy plasmids with an alpha-mating factor sequence (for secretion). The cDNA for the oxidation-resistant ApoA1 variant polypeptide monomer can be codon-optimized for high-level expression in yeast. High-copy plasmid expression vectors optimized for use with proprietary yeast strains (for example in S. cerevisiae) are commercially available. The plasmid DNA can be transformed into super-secretory yeast strains that allows secretion of 90% of proteins into media during fermentation. The construct can illustratively also contain a purification or identity tag, as detailed above, to simply purification by affinity chromatography, for example, immobilized metal ion affinity chromatography (IMAC). This technique can provide protein of purity ranging from greater than 70%, for example, greater than 70%, or greater than 75%, or greater than 80%, or greater than 85%, or greater than 90%, or greater than 95%, to about 96-100% homogeneous, and if further purification is required, ion-exchange and size exclusion chromatography can be employed singly or in combination, methods which are routinely used in the protein purification field.

Several well-established methodologies for preparing specific polypeptides using recombinant DNA technology are available to those skilled in the art. For example, several established books and literature reviews provide teachings on how to clone and produce recombinant proteins using vectors and prokaryotic or eukaryotic host cells, such as some titles in the series “A Practical Approach” published by Oxford University Press (DNA Cloning 2: Expression Systems, (1995); DNA Cloning 4: Mammalian Systems, (1996); Protein Expression, (1999); Protein Purification Techniques, (2001)).

In some embodiments, a useful method for producing recombinant oxidation-resistant ApoA1 variant polypeptide monomers includes insertion of a polynucleotide sequence operable to encode an oxidation-resistant ApoA1 variant polypeptide monomer of SEQ ID NOs:3-33 or a polypeptide having at least 70%, or at least 75%, or at least 80%, or at least 85%, or at least 90%, or at least 95% sequence identity to any one of SEQ ID NOs:3-33, into a transposon-based vector employing a promoter and other regulatory sequences operably-linked to the transposase gene derived from a specific host. In some embodiments, the vector contains a polynucleotide comprising a polynucleotide sequence as provided in SEQ ID NO:34. In some embodiments, host specific regulatory sequences can be tissue specific or can be of a constitutive nature. Illustrative methods for (1) constructing the transposon-based vector containing a polynucleotide sequence operable to encode a polypeptide (for example, an oxidation-resistant ApoA1 variant polypeptide monomer of SEQ ID NO:3-33, or for example, the polynucleotide shown in SEQ ID NO:34); (2) methods for transfecting a host cell with the transposon-based vector to express a polypeptide of interest (for example, an oxidation-resistant ApoA1 variant polypeptide monomer of SEQ ID NO:3-33) in a host cell; and (3) methods for producing the polypeptide (for example, an oxidation-resistant ApoA1 variant polypeptide monomer of SEQ ID NO:3-33) in a host cell are described and exemplified in U.S. Patent Application Publication No. 2010/0261227 (Ser. No. 12/757,591) filed on Apr. 9, 2010, said patent reference is incorporated herein by reference in its entirety.

In some embodiments, methods for isolating or purifying oxidation-resistant ApoA1 variant polypeptide monomers, utilized by the invention (for example, polypeptides having an amino acid sequence of SEQ ID NO:3-33 or functional fragments thereof). For example, oxidation-resistant ApoA1 variant polypeptide monomers may be purified using ammonium sulfate or ethanol precipitation, gel filtration, hydrophobic interaction chromatography, size-exclusion chromatography, affinity chromatography, hydroxylapatite chromatography, and high performance liquid chromatography among others. Art-recognized techniques for the purification of proteins and peptides are set forth, for example, in Methods in Enzymology, “Guide to Protein Purification,” Murray P. Deutscher, ed. (1990) Vol. 182.

In some embodiments, the isolation and purification of an oxidation-resistant ApoA1 variant polypeptide monomer can include selecting clones expressing the oxidation-resistant ApoA1 variant polypeptide monomer, for example, a polypeptide having an amino acid sequence of SEQ ID NO:5 in an avian hepatoma line and growing the selected avian hepatoma cell line expressing the recombinant oxidation-resistant ApoA1 variant polypeptide monomer of interest in a hollow fiber perfusion culture system. An advantage of this system is the long-term production of the protein. Cells expressing an oxidation-resistant ApoA1 variant polypeptide monomer can be maintained in culture and produced for 10-200 days, with weekly harvest that allows weekly purification of the oxidation-resistant ApoA1 variant polypeptide monomer of interest.

In some embodiments, recombinant oxidation-resistant ApoA1 variant polypeptide monomers can be separated from a culture medium (e.g. bacterial, cell, plant or insect), for example, by density gradient centrifugation followed by any down-stream processing steps, such as gel-filtration chromatography, ion-exchanging chromatography, hydrophobic, e.g., phenyl sepharose, interaction chromatography or immunoaffinity chromatography known to those skilled in the art and subsequent purification familiar to those skilled in the art. (See, e.g., U.S. Pat. Nos. 6,107,467; 6,559,284; 6,423,830; 6,090,921; 5,834,596; 5,990,081; 6,506,879, Mulugeta et al., J. Chromatogr. (1998) 798(1-2):83-90; Chung et al., J. Lipid Res. (1980) 21(3):284-91; Cheung et al., J. Lipid Res. (1987) 28(8):913-29; Persson, et al., J. Chromatogr. (1998) 711:97-109; U.S. Pat. Nos. 5,059,528, 5,834,596, 5,876,968 and 5,721,114; and PCT Publications WO 86/04920 and WO 87/02062).

The oxidation-resistant ApoA1 variant polypeptide monomers can be purified and isolated as described above and known in the art of protein purification of apolipoproteins. The purified oxidation-resistant ApoA1 variant polypeptide monomers can then be resuspended in physiological buffers, to enable dimerization of the monomer polypeptide subunits into dimers useful in the treatment of dyslipidemia and cardiovascular diseases in a subject in need thereof. In some embodiments, dimerization of the purified oxidation-resistant ApoA1 variant polypeptide monomers can occur by the natural association of two monomers under favorable conditions. The dimerization may also be induced by chemical oxidation processes known in the art, including metal catalyzed oxidation of cysteines to form a disulfide cystine bridge. In other embodiments, oxidation of the purified oxidation-resistant ApoA1 variant polypeptide monomers into dimers can be accomplished by incubating the monomers in tris buffer pH 9.0 containing 1.5-6 M guanidine-HCl in the presence of reduced glutathione/oxidized glutathione with and without thioredoxin as described in Pigiet V P et al., Proc. Natl. Acad. Sci., USA, (1989) 83:7643-7647). Dimer formation can be monitored using reducing and non-reducing PAGE and analytical high performance size exclusion chromatography (HPSEC). In some embodiments, methods for inducing dimerization of the purified oxidation-resistant ApoA1 variant polypeptide monomers can occur using copper(II) catalysis of the singe cysteine residue in each monomer as described in Cavallini, D., et al., Arch. Biochem. and Biophys. (1969) 130:354-361, and is incorporated herein in its entirety

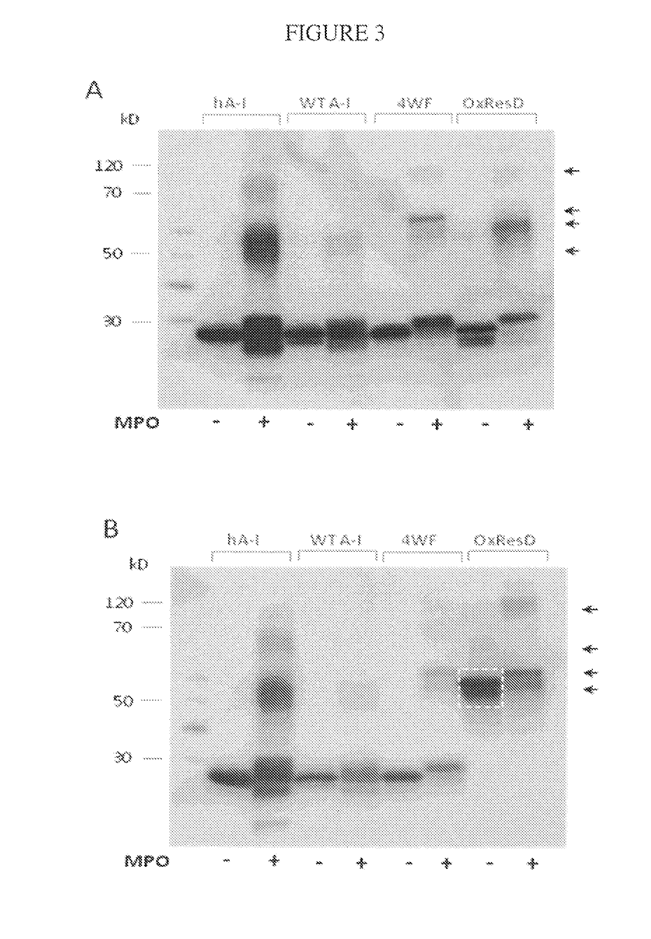

As provided herein, the oxidation-resistant ApoA1 variant polypeptide monomer subunits naturally form covalent bonds between the cysteine residues present in each oxidation-resistant ApoA1 variant polypeptide monomer subunit. As shown in FIG. 3, the oxidation-resistant ApoA1 variant polypeptide monomer subunits form stable dimers that are resistant to degradation by oxidation by oxidizing agents.

### Lipid Complexes Comprising Oxidation-Resistant ApoA1 Variant Monomers, Dimers or Oxidation-Resistant ApoA1 Monomers

In certain embodiments, the compositions and methods of the invention comprise administration of lipid complexes having at least one of: oxidation-resistant ApoA1 variant polypeptide monomers, oxidation-resistant ApoA1 variant dimers. In some embodiments, the compositions and methods of the invention comprise administration of lipid complexes containing an oxidation-resistant ApoA1 monomer (SEQ ID NO:35). In some embodiments, the invention provides formulations of oxidation-resistant ApoA1 variant monomer or dimer: lipid complexes. In some embodiments, the invention provides formulations comprising lipid complexes of oxidation-resistant ApoA1 monomers. Efficacy can be enhanced by the complexing of lipids to oxidation-resistant ApoA1 variant monomers or dimers of the present invention. In some embodiments, efficacy can be enhanced by the complexing of lipids to oxidation-resistant ApoA1 monomers of the present invention. Typically, the lipid is mixed with the oxidation-resistant ApoA1 variant monomers or dimers. In some embodiments, the lipid is mixed with an oxidation-resistant ApoA1 monomer prior to administration. Oxidation-resistant ApoA1 variant monomers or dimers, and lipids can be mixed in an aqueous solution in appropriate ratios and can be complexed by methods known in the art including freeze-drying, detergent solubilization followed by dialysis, microfluidization, sonication, and homogenization. In further embodiments, oxidation-resistant ApoA1 monomers and lipids can be mixed in an aqueous solution in appropriate ratios and can be complexed by methods known in the art including freeze-drying, detergent solubilization followed by dialysis, microfluidization, sonication, and homogenization. Complex efficiency can be optimized, for example, by varying pressure, ultrasonic frequency, or detergent concentration. An example of a detergent commonly used to prepare lipid complexes of oxidation-resistant ApoA1 variant monomers or dimers is sodium cholate. In another embodiment, an example of a detergent commonly used to prepare lipid complexes of oxidation-resistant ApoA1 monomer is sodium cholate.

In some embodiments, the oxidation-resistant ApoA1 variant monomer or dimer: lipid complex is administered to a subject in need thereof in therapeutically effective quantities and dosing schedules readily ascertainable by a medical professional. In some embodiments, the oxidation-resistant ApoA1 monomer: lipid complex is administered to a subject in need thereof in therapeutically effective quantities and dosing schedules readily ascertainable by a medical professional.

In one embodiment, the oxidation-resistant ApoA1 variant monomer or dimers: lipid complex can be in solution with an appropriate pharmaceutical diluent. In another embodiment, freeze-dried or lyophilized preparations of oxidation-resistant ApoA1 variant monomer or dimers or oxidation-resistant ApoA1 monomers: lipid complexes can be reconstituted or rehydrated with phosphate buffered saline, a physiological saline solution or some other appropriate pharmaceutical diluent prior to administration. In another embodiment, the oxidation-resistant ApoA1 monomer: lipid complex can be in solution with an appropriate pharmaceutical diluent. In another embodiment, freeze-dried or lyophilized preparations of oxidation-resistant ApoA1 variant monomer or dimers: lipid complexes can be reconstituted or rehydrated with phosphate buffered saline, a physiological saline solution or some other appropriate pharmaceutical diluent prior to administration. In another embodiment, freeze-dried or lyophilized preparations of oxidation-resistant ApoA1 monomers: lipid complexes can be reconstituted or rehydrated with phosphate buffered saline, a physiological saline solution or some other appropriate pharmaceutical diluent prior to administration.

In another embodiment, the oxidation-resistant ApoA1 variant monomer or dimer: lipid complexes can be frozen preparations that are thawed until a homogenous solution is achieved prior to administration to a subject in need thereof. In another embodiment, the oxidation-resistant ApoA1 monomer: lipid complexes can be frozen preparations that are thawed until a homogenous solution is achieved prior to administration to a subject in need thereof. The lipid can be any suitable lipid known to those of skill in the art. Non-phosphorus containing lipids can be used, including stearylamine, dodecylamine, acetyl palmitate, (1,3)-D-mannosyl-(1,3)diglyceride, aminophenylglycoside, 3-cholesteryl-6′-(glycosylthio)hexyl ether glycolipids, N-(2,3-di(9-(Z)-octadecenyloxy))-prop-1-yl-N,N,N-trimethylammonium chloride and fatty acid amides.

In some embodiments, the lipid used in the oxidation-resistant ApoA1 variant monomer or dimer: lipid complex can include a phospholipid. In other embodiments, the lipid used in the oxidation-resistant ApoA1 monomer: lipid complex can include a phospholipid. Useful phospholipids for preparing the oxidation-resistant ApoA1 variant monomer or dimer: lipid complexes can include any one or more of: phosphatidylcholine, phosphatidylserine, phosphatidylethanolamine, phosphatidic acid, phosphatidylinositol, and sphingomyelin. Useful phospholipids for preparing the oxidation-resistant ApoA1 monomer: lipid complex can include any one or more of: phosphatidylcholine, phosphatidylserine, phosphatidylethanolamine, phosphatidic acid, phosphatidylinositol, and sphingomyelin.

In some embodiments, the phospholipid can be any phospholipid known to those of skill in the art. For example, the phospholipid can be a small alkyl chain phospholipid, phosphatidylcholine, egg phosphatidylcholine, soybean phosphatidylcholine, dipalmitoylphosphatidylcholine, soy phosphatidylglycerol, egg phosphatidylglycerol, distearoylphosphatidylglycerol, dimyristoylphosphatidylcholine, distearoylphosphatidylcholine, dilaurylphosphatidylcholine, 1-myristoyl-2-palmitoylphosphatidylcholine, 1-palmitoyl-2-myristoylphosphatidylcholine, 1-palmitoyl-2-stearoylphosphatidylcholine, 1-stearoyl-2-palmitoylphosphatidylcholine, dioleoylphosphatidylcholine, 1-palmitoyl-2-oleoylphosphatidylcholine, 1-oleoyl-2-palmitylphosphatidylcholine, dioleoylphosphatidylethanolamine, dilauroylphosphatidylglycerol, phosphatidylserine, phosphatidylethanolamine, phosphatidylinositol, phosphatidylglycerol, diphosphatidylglycerol, dimyristoylphosphatidylglycerol, dipalmitoylphosphatidylglycerol, distearoylphosphatidylglycerol, dioleoylphosphatidylglycerol, phosphatidic acid, dimyristoylphosphatidic acid, dipalmitoylphosphatidic acid, dimyristoylphosphatidylethanolamine, dipalmitoylphosphatidylethanolamine, dimyristoylphosphatidylserine, dipalmitoylphosphatidylserine, brain phosphatidylserine, sphingomyelin, sphingolipids, brain sphingomyelin, dipalmitoylsphingomyelin, distearoylsphingomyelin, galactocerebroside, gangliosides, cerebrosides, phosphatidylglycerol, phosphatidic acid, lysolecithin, lysophosphatidylethanolamine, cephalin, cardiolipin, dicetylphosphate, distearoyl-phosphatidylethanolamine and cholesterol and its derivatives.

The phospholipid can also be a derivative or analog of any of the above phospholipids. In certain embodiments, the oxidation-resistant ApoA1 variant dimer: phospholipid complex can comprise combinations of two or more phospholipids. In certain embodiments, the oxidation-resistant ApoA1 variant polypeptide monomer: phospholipid complex can comprise combinations of two or more phospholipids.

In certain embodiments, the oxidation-resistant ApoA1 monomer: phospholipid complex can comprise combinations of two or more phospholipids.

To further enhance the stability of the polypeptide: phospholipid complex, homogeneity of the polypeptide: phospholipid complex, and/or increase the antioxidant and other favorable properties, additional agents, such as APOA2, lutein, beta-carotene, lycopene, paraoxonase, flavones, flavonoids, hydrophobic agents, and hydrophobic anti-oxidant agents may be added to the polypeptide: phospholipid complex.

In one embodiment, the lipid is a phospholipid, preferably, 1-palmitoyl-2-oleoyl phosphatidylcholine (“POPC”) or (“1-palmitoyl-2-oleoyl-sn-glycero-3-phosphocholine”). In another embodiment, the lipid is a phospholipid, preferably, dipalmitoylphosphatidylcholine (“DPPC”). In some embodiments, the present invention provides an oxidation-resistant ApoA1 variant monomer: phospholipid complex. In other embodiments, the present invention provides an oxidation-resistant ApoA1 variant dimer: phospholipid complex. In one embodiment, the lipid is a phospholipid, preferably, 1-palmitoyl-2-oleoyl phosphatidylcholine (“POPC”) or (“1-palmitoyl-2-oleoyl-sn-glycero-3-phosphocholine”). In another embodiment, the lipid is a phospholipid, preferably, dipalmitoylphosphatidylcholine (“DPPC”).

In other embodiments, the present invention provides an oxidation-resistant ApoA1 monomer: phospholipid complex. In one embodiment, the lipid is a phospholipid, preferably, 1-palmitoyl-2-oleoyl phosphatidylcholine (“POPC”) or (“1-palmitoyl-2-oleoyl-sn-glycero-3-phosphocholine”). In another embodiment, the lipid is a phospholipid, preferably, dipalmitoylphosphatidylcholine (“DPPC”).

In another embodiment, the oxidation-resistant ApoA1 variant monomer or dimer: POPC complex comprises from about a 1 to 1 to about a 1 to 300 molar ratio of oxidation-resistant ApoA1 variant monomer or dimer: POPC. In another embodiment, the oxidation-resistant ApoA1 monomer:POPC complex comprises from about a 1 to 1 to about a 1 to 300 molar ratio of oxidation-resistant ApoA1 monomer:POPC.

In other embodiments, the oxidation-resistant ApoA1 variant monomer or dimer: POPC complex comprises from about 1 to 3 to about 1 to 200 molar ratio of oxidation-resistant ApoA1 variant monomer or dimer or oxidation-resistant ApoA1 monomer:POPC. In other embodiments, the oxidation-resistant ApoA1 monomer:POPC complex comprises from about 1 to 3 to about 1 to 200 molar ratio of oxidation-resistant ApoA1 monomer:POPC.

In further embodiments, the molar ratio of oxidation-resistant ApoA1 variant monomer or dimer: POPC comprises 1 to 37, 1 to 110-120, or from 1 to 120-150 ratio of polypeptide to phospholipid. In further embodiments, the molar ratio of oxidation-resistant ApoA1 monomer:POPC comprises 1 to 37, 1 to 110-120, or from 1 to 120-150 ratio of polypeptide to phospholipid. In some embodiments, a preferred ratio of oxidation-resistant ApoA1 variant monomer or dimer: POPC comprises 1 to 110 selected to produce a homogenous, stable and reproducible population of polypeptide: phospholipid complexes. In some embodiments, a preferred ratio of oxidation-resistant ApoA1 monomer:POPC comprises 1 to 110 selected to produce a homogenous, stable and reproducible population of polypeptide: phospholipid complexes. In an even more preferred embodiment, the molar ratio of oxidation-resistant ApoA1 variant monomer or dimer to phospholipid is 1:111 (moles of protein/moles of lipid). In an even more preferred embodiment, the molar ratio of oxidation-resistant ApoA1 monomer to phospholipid is 1:111 (moles of protein/moles of lipid).

In another embodiment, the oxidation-resistant ApoA1 variant monomer or dimer: DPPC complex comprises from about 1 to 1 to about a 1 to 300 molar ratio of oxidation-resistant ApoA1 variant monomer or dimer: DPPC. In another embodiment, the oxidation-resistant ApoA1 monomer:DPPC complex comprises from about 1 to 1 to about a 1 to 300 molar ratio of oxidation-resistant ApoA1 monomer:DPPC. In other embodiments, the oxidation-resistant ApoA1 variant monomer or dimer: DPPC complex comprises from about 1 to 3 to about a 1 to 200 molar ratio by weight of oxidation-resistant ApoA1 variant monomer or dimer: DPPC. In other embodiments, the oxidation-resistant ApoA1 monomer:DPPC complex comprises from about 1 to 3 to about a 1 to 200 molar ratio by weight of oxidation-resistant ApoA1 monomer:DPPC.

In further embodiments, the molar ratio of oxidation-resistant ApoA1 variant monomer or dimer: DPPC comprises 1 to 37, 1 to 110-120, or from 1 to 120-150. In one embodiment, the oxidation-resistant ApoA1 variant monomer or dimer: POPC complex is a pharmaceutical formulation. In further embodiments, the molar ratio of oxidation-resistant ApoA1 monomer:DPPC comprises 1 to 37, 1 to 110-120, or from 1 to 120-150. In one embodiment, the oxidation-resistant ApoA1 monomer:POPC complex is a pharmaceutical formulation.

In other embodiments, the oxidation-resistant ApoA1 monomer:POPC complex comprises from about 1 to 3 to about 1 to 200 molar ratio of oxidation-resistant ApoA1 monomer:POPC. In other embodiments, the oxidation-resistant ApoA1 monomer:POPC complex comprises from about 1 to 3 to about 1 to 200 molar ratio of oxidation-resistant ApoA1 monomer:POPC.

Additional lipids suitable for use in the methods of the invention are well known to persons of skill in the art and are cited in a variety of well known sources, e.g., McCutcheon's Detergents and Emulsifiers and McCutcheon's Functional Materials, Allured Publishing Co., Ridgewood, N.J., both of which are incorporated herein by reference. Generally, it is desirable that the lipids are liquid-crystalline at 37° C., 35° C., or 32° C. Lipids in the liquid-crystalline state typically accept cholesterol more efficiently than lipids in the gel state. As subjects typically have a core temperature of about 37° C., lipids that are liquid-crystalline at 37° C. are generally in a liquid-crystalline state during treatment.

Methods for preparing apolipoprotein-phospholipid complexes are well known in the art, such that the oxidation-resistant ApoA1 variant monomer or dimer: lipid complexes can be made by any method known to one of skill in the art. Other illustrative methods for preparing apolipoprotein-phospholipid complexes are well known in the art, such that the oxidation-resistant ApoA1 monomer: lipid complexes can be made by any method known to one of skill in the art. In some cases it is desirable to mix the lipid and oxidation-resistant ApoA1 variant monomer or dimer: lipid complex prior to administration. In some cases it is desirable to mix the lipid and oxidation-resistant ApoA1 monomer: lipid complex prior to administration.

Protein-lipid complexes can be formed using standard techniques such as high pressure homogenization, microfluidization sonication or extrusion. For example, oxidation-resistant ApoA1 variant monomer or dimer can be co-sonicated (using a bath or probe sonicator) with the appropriate lipid to form protein lipid complexes. In other embodiments, oxidation-resistant ApoA1 monomer can be co-sonicated (using a bath or probe sonicator) with the appropriate lipid to form protein lipid complexes.

In another embodiment, the oxidation-resistant ApoA1 variant monomer or dimer can also be made by a detergent dialysis method; e.g., a mixture of oxidation-resistant ApoA1 variant monomer or dimer, lipid and a detergent such as cholate can be dialyzed to remove the detergent and reconstituted to make the lipid complexes. In another embodiment, the oxidation-resistant ApoA1 monomer lipid complex can also be made by a detergent dialysis method; e.g., a mixture of oxidation-resistant ApoA1 monomer, lipid and a detergent such as cholate can be dialyzed to remove the detergent and reconstituted to make the lipid complexes. (See, e.g., Jonas et al., Methods Enzymol. (1986) 128:553-82). In another embodiment, the oxidation-resistant ApoA1 variant monomer or dimer-lipid complexes can be made by co-lyophilization, as described in U.S. Pat. Nos. 6,287,590 and 6,455,088, the contents of which are hereby incorporated by reference in their entirety. In another embodiment, the oxidation-resistant ApoA1 monomer—lipid complexes can be made by co-lyophilization, as described in U.S. Pat. Nos. 6,287,590 and 6,455,088, the contents of which are hereby incorporated by reference in their entireties. Other methods are disclosed, for example, in U.S. Pat. Nos. 6,004,925, 6,037,323, 6,046,166, and 6,306,433 which are all incorporated herein by reference in their entireties. Other methods of preparing oxidation-resistant ApoA1 variant monomer or dimer: phospholipid complexes will be apparent to those of skill in the art. In some embodiments, other methods of preparing oxidation-resistant ApoA1 monomer: phospholipid complexes will be apparent to those of skill in the art.

In one embodiment, a method of preparing phospholipid complexes containing oxidation-resistant ApoA1 variant monomer or dimer, or oxidation-resistant ApoA1 monomer provides useful phospholipid complexes that are not liposomal or proteoliposomal in nature.

In an illustrative embodiment, a method for making an oxidation-resistant ApoA1 variant monomer, or dimer, or ApoA1 lipid complexes can include: adding a phospholipid for example, POPC or DPPC to a solution of recombinantly produced oxidation-resistant ApoA1 variant monomer, or dimer, or ApoA1 lipid, in 10 mM sodium phosphate (pH 7.5). The material can be pre-mixed for 10 minutes at 50° C. with an Ystral X 20 D-mix equipment. The material can then be homogenized at 42° C. and 900 bar in a high pressure homogenization of the type Lab. Rannie 12.51-H. The homogenization can be performed for 10-50 passages where each passage has duration of 1-5 minutes. After the homogenization sucrose and mannitol can be added and dissolved, whereupon the solution is subsequently sterile filtered, aseptically filled and freeze-dried. Subsequent electrophoretic and chromatographic analyzes of the product after its reconstitution showed that the protein was in compliance with the protein material not subjected to homogenization. Analyses demonstrated with the IEF data of a sample of the homogenized, final protein preparation according to this example in comparison to an untreated protein. The efficacy of the process is shown by n-PAGE tests where 99% of the protein was incorporated into lipoprotein particles in the size range 6 to 25 nm.

In one embodiment, the lipid complexes of the present invention can be made by homogenization. In other embodiments, the making of oxidation-resistant ApoA1 variant monomer or dimer: lipid complexes begin when recombinant oxidation-resistant ApoA1 variant monomer or dimer or oxidation-resistant ApoA1 monomer is diluted to a concentration of 10 mg/ml in solution with water for injection. In one embodiment, the lipid complexes can be made by homogenization. In other embodiments, the making of oxidation-resistant ApoA1 monomer: lipid complexes begin when recombinant oxidation-resistant ApoA1 monomer is diluted to a concentration of 10 mg/ml in solution with water for injection. Sodium phosphate is added to a final concentration of 9-15 mM phosphate and to adjust the pH to between about 7.0 and about 7.4. Mannitol is added to achieve a concentration of about 0.8% to about 1% mannitol (w/v). Then POPC is added to achieve a mixture of about 1:118 (molar ratio of protein/wt lipid) of oxidation-resistant ApoA1 variant monomer or dimer to POPC. In other embodiments, POPC is added to achieve a mixture of about 1:118 (molar ratio of protein/wt lipid) of oxidation-resistant ApoA1 monomer to POPC. The mixture is stirred at 1500-2000 rpm for about 10 minutes using an overhead propeller and an Ultra Turrax while maintaining the temperature between about 37° C. to about 40° C. The feed vessel is stirred continuously at 1500 rpm while the temperature is maintained between 32° to 40°. Homogenization for the first 5 minutes can be carried out at 0.5 MPa (7,700 psi) keeping the temperature between 45-55°, and thereafter, the pressure is maintained at 1 MPa (14,500 psi) until in-process testing by size-exclusion chromatography/native PAGE demonstrates the % AUC of >70% between protein standards. The complexes may also be made as 2.5 mg/mL, 5.0 mg/mL, 10.0 mg/mL, 13 mg/mL, 14 mg/mL, 15 mg/mL, 16 mg/mL, 17 mg/mL, 18 mg/mL, 19 mg/mL, 20 mg/mL, or from about 21 mg/mL to about 50 mg/mL (w/v) formulations wherein the weight is that of protein.

### Pharmaceutical Compositions and Formulations

Monomers or dimers of oxidation-resistant ApoA1 variant polypeptides, or lipid complexed monomers or dimers of oxidation-resistant ApoA1 variant polypeptides or functional fragments thereof and/or pharmaceutically acceptable salts thereof, can be used in pharmaceutical compositions of the present invention. In some embodiments, lipid complexed monomers of oxidation resistant ApoA1 comprising the amino acid sequence of SEQ ID NO:35), or functional fragments thereof and/or pharmaceutically acceptable salts thereof, can be used in pharmaceutical compositions of the present invention. In some embodiments, monomers or dimers of oxidation-resistant ApoA1 variant polypeptides, or lipid complexed monomers or dimers of oxidation-resistant ApoA1 variant polypeptides can be incorporated into a pharmaceutical composition suitable for administration to cure, treat or prevent any disease or disorder that would benefit from enhanced cholesterol efflux from lipid laden cells or reduce inflammatory activity.

In some embodiments, lipid complexed monomers of oxidation resistant ApoA1 can be incorporated into a pharmaceutical composition suitable for administration to cure, treat or prevent any disease or disorder that would benefit from enhanced cholesterol efflux from lipid laden cells or reduce inflammatory activity. In some embodiments, monomers or dimers of oxidation-resistant ApoA1 variant polypeptides, or lipid complexed monomers or dimers of oxidation-resistant ApoA1 variant polypeptides or lipid complexed monomers of oxidation resistant ApoA1 can be incorporated into a pharmaceutical composition suitable for administration to cure, treat or prevent any disease or disorder that would benefit from enhanced cholesterol efflux from lipid laden cells or reduce inflammatory activity in blood vessels. In some embodiments, lipid complexed monomers of oxidation resistant ApoA1 can be incorporated into a pharmaceutical composition suitable for administration to cure, treat or prevent any disease or disorder that would benefit from enhanced cholesterol efflux from lipid laden cells or reduce inflammatory activity in blood vessels. In some embodiments, the disease or disorder can be a cardiovascular or dyslipidemic disorder, or disease or related symptoms. Such compositions typically comprise purified monomers or dimers of oxidation-resistant ApoA1 variant polypeptides, or lipid complexed monomers or dimers of oxidation-resistant ApoA1 variant polypeptides and a pharmaceutically acceptable carrier, diluent or excipient. In some embodiments, the disease or disorder can be a cardiovascular or dyslipidemic disorder, or disease or related symptoms. Such compositions typically comprise purified lipid complexed monomers of oxidation resistant ApoA1 and a pharmaceutically acceptable carrier, diluent or excipient. As used herein, “pharmaceutically acceptable carrier, diluent or excipient” is intended to include any and all solvents, dispersion media, coatings, antibacterial and anti-fungal agents, isotonic and absorption-delaying agents, and the like, that are compatible with pharmaceutical administration. The use of such media and agents for pharmaceutically active substances is well known in the art. Except insofar as any conventional media or agent is incompatible with the active monomers or dimers of oxidation-resistant ApoA1 variant polypeptides, or lipid complexed monomers or dimers of oxidation-resistant ApoA1 variant polypeptides or lipid complexed monomers of oxidation resistant ApoA1, the carrier, diluent or excipient, use thereof of such media or agents in the pharmaceutical composition is contemplated. Except insofar as any conventional media or agent is incompatible with the lipid complexed monomers of oxidation resistant ApoA1, the carrier, diluent or excipient, use thereof of such media or agents in the pharmaceutical composition is contemplated.

In some embodiments, pharmaceutical compositions and pharmaceutical formulations will comprise, at least one pharmaceutically acceptable excipient, carrier or vehicle, and monomers or dimers of oxidation-resistant ApoA1 variant polypeptides, or lipid complexed monomers or dimers of oxidation-resistant ApoA1 variant polypeptides or lipid complexed monomers of oxidation resistant ApoA1 of the present invention complexed with a phospholipid, for example, POPC and/or DPPC as exemplified above. In some embodiments, pharmaceutical compositions and pharmaceutical formulations will comprise, at least one pharmaceutically acceptable excipient, carrier or vehicle, and lipid complexed monomers of oxidation resistant ApoA1 of the present invention complexed with a phospholipid, for example, POPC and/or DPPC as exemplified above.

A pharmaceutical composition of the present invention can be formulated to be compatible with its intended route of administration, as determined by those of skill in the art, and optionally, formulated under FDA-approved methods. Exemplary routes of administration of monomers or dimers of oxidation-resistant ApoA1 variant polypeptides, or lipid complexed monomers or dimers of oxidation-resistant ApoA1 variant polypeptides or lipid complexed monomers of oxidation resistant ApoA1 include: parenteral, oral (e.g., ingestion or inhalation); transdermal (topical), subcutaneous, and transmucosal administration. In some embodiments, exemplary routes of administration of lipid complexed monomers of oxidation resistant ApoA1 include: parenteral, oral (e.g., ingestion or inhalation); transdermal (topical), subcutaneous, and transmucosal administration. In one embodiment, subcutaneous administration of naked or non-complexed monomers or dimers of oxidation-resistant ApoA1 variant polypeptides are intended for administration to a subject having a disease or disorder. In such an embodiment, it is intended that the route of administration will enable the naked or non-complexed monomers or dimers of oxidation-resistant ApoA1 variant polypeptides to travel to the lymph system where it can be further distributed to the vasculature. The diseases and disorders contemplated can include: Alzheimer's disease, cancer, for example, prostate cancer, breast cancer or colon cancer, cardiovascular diseases, diabetic nephropathy, diabetic retinopathy, disorders of glucose metabolism, dyslipidemia, dyslipoproteinemia, hypertension, impotence, inflammation, rheumatoid arthritis, insulin resistance disorders, lipid elimination in bile, obesity, oxysterol elimination in bile, pancreatitis (pancreatitus) Parkinson's disease, peroxisome proliferator activated receptor (PPAR)-associated disorders, phospholipid elimination in bile, renal diseases, septicemia, metabolic syndrome disorders, thrombotic disorders, C-reactive protein imbalance and insufficient bile production. Solutions or suspensions used for parenteral, intradermal, or subcutaneous application can include one or more of the following components: a sterile diluent such as water for injection, saline solution (e.g., phosphate buffered saline (PBS)), fixed oils, a phospholipid, a polyol (for example, glycerol, propylene glycol, and liquid polyethylene glycol, and the like), glycerine, or other synthetic solvents; antibacterial and antifungal agents such as parabens, chlorobutanol, phenol, ascorbic acid, thimerosal, and the like; antioxidants such as ascorbic acid or sodium bisulfite; chelating agents such as ethylenediaminetetraacetic acid (EDTA); buffers such as acetates, citrates or phosphates and agents for the adjustment of tonicity such as sodium chloride or dextrose. The proper fluidity can be maintained, for example, by the use of a coating such as phospholipid, by the maintenance of the required particle size in the case of dispersion and by the use of surfactants. In many cases, it will be preferable to include isotonic agents, for example, sugars, polyalcohols such as mannitol or sorbitol, and sodium chloride in the composition. Prolonged administration of the injectable compositions can be brought about by including an agent that delays absorption. Such agents include, for example, aluminum monostearate and gelatin. The parenteral preparation can be enclosed in ampoules, disposable syringes or multiple dose vials made of glass or plastic.

Oral compositions generally include an inert diluent or an edible carrier. Oral compositions can be liquid, or can be enclosed in gelatin capsules or compressed into tablets. Generally, oxidation-resistant ApoA1 variant monomers or dimers of the present invention can be administered naked or preferably in lipid complexes, for example, in phospholipid complexes. In some embodiments, monomers or dimers of oxidation-resistant ApoA1 variant polypeptides, or lipid complexed monomers or dimers of oxidation-resistant ApoA1 variant polypeptides can be formulated for oral compositions. In some embodiments, the lipid can be a phospholipid. In some embodiments, lipid complexed monomers of oxidation resistant ApoA1 can be formulated for oral compositions. In some embodiments, the lipid can be a phospholipid. As such, oral administration, whether by ingestion, buccal or sublingual routes, generally requires appropriate formulations, such as enteric coatings that are operable to minimize degradation of the active agents as a result of passage through the oral mucosa, stomach and small intestine. Pharmaceutically compatible binding agents, and/or adjuvant materials can be included as part of an oral composition. Tablets, pills, capsules, troches and the like can contain any of the following ingredients, or compounds of a similar nature: a binder such as microcrystalline cellulose, gum tragacanth or gelatin; an excipient such as starch or lactose; a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring. Transmucosal administration can be accomplished through the use of nasal sprays or suppositories. For transdermal administration, the active compounds are formulated into ointments, salves, gels, or creams as generally known in the art.

It is especially advantageous to formulate oral, parenteral, transmucosal or transdermal compositions in dosage unit form for ease of administration and uniformity of dosage. A “dosage unit form” as used herein refers to physically discrete units suited as unitary dosages for an individual to be treated; each unit containing a predetermined quantity of active compound calculated to produce the desired therapeutic effect in association with the required pharmaceutical carrier. The dosage unit forms of the present invention are dependent upon the amount of a compound necessary to therapeutically treat the individual. The amount of a compound necessary can be formulated in a single dose, or can be formulated in multiple dosage units. Treatment of an individual may require a one-time dose, or may require repeated doses.

The compositions are usually employed in the form of pharmaceutical preparations. Such preparations are made in a manner well known in the pharmaceutical art. One preferred preparation utilizes a vehicle of physiological saline solution, but it is contemplated that other pharmaceutically acceptable carriers such as physiological concentrations of other non-toxic salts, five percent aqueous glucose solution, sterile water or the like may also be used. As used herein “pharmaceutically acceptable carrier” includes any and all solvents, dispersion media, coatings, antibacterial and antifungal agents, isotonic and absorption delaying agents, and the like. The use of such media and agents for pharmaceutically active substances is well known in the art. Except insofar as any conventional media or agent is incompatible with the active monomers or dimers of oxidation-resistant ApoA1 variant polypeptides, or lipid complexed monomers or dimers of oxidation-resistant ApoA1 variant polypeptides, use thereof in the therapeutic compositions is contemplated. In some embodiments, except insofar as any conventional media or agent is incompatible with the lipid complexed monomers of oxidation resistant ApoA1, use thereof in the therapeutic compositions is contemplated. Supplementary or additional active agents can also be incorporated into the compositions, for example, cardiovascular active agents, anti-inflammatory agents, anti-hypertension agents, immunosuppressive agents and are described in greater detail below. It may also be desirable that a suitable buffer be present in the composition. Such solutions can, if desired, be lyophilized and stored in a sterile ampoule ready for reconstitution by the addition of sterile water for ready injection. The primary solvent can be aqueous or alternatively non-aqueous. Oxidation-resistant ApoA1 variant monomers or dimers can also be incorporated into a solid or semi-solid biologically compatible matrix which can be implanted into tissues requiring treatment.

The carrier can also contain other pharmaceutically-acceptable excipients for modifying or maintaining the pH, osmolarity, viscosity, clarity, color, sterility, stability, rate of dissolution, or odor of the formulation. Similarly, the carrier may contain still other pharmaceutically-acceptable excipients for modifying or maintaining release or absorption or penetration across the blood-brain barrier. Such excipients are those substances usually and customarily employed to formulate dosages for parenteral administration in either unit dosage or multi-dose form or for direct infusion by continuous or periodic infusion.

The compositions may be formulated so as to provide rapid, sustained, or delayed release of the active monomers or dimers of oxidation-resistant ApoA1 variant polypeptides, or lipid complexed monomers or dimers of oxidation-resistant ApoA1 variant polypeptides or lipid complexed monomers of oxidation resistant ApoA1 after administration to the patient by employing procedures well known in the art. In further embodiments, the compositions may be formulated so as to provide rapid, sustained, or delayed release of the active lipid complexed monomers of oxidation resistant ApoA1 after administration to the patient by employing procedures well known in the art. The formulations can also contain substances that diminish proteolytic degradation and/or substances, which promote absorption such as, for example, surface-active agents.

### Methods of Administration

In some embodiments, pharmaceutical compositions and formulations comprise monomers or dimers of oxidation-resistant ApoA1 variant polypeptides, or lipid complexed monomers or dimers of oxidation-resistant ApoA1 variant polypeptides. In one embodiment, pharmaceutical compositions and formulations comprise lipid complexed monomers of oxidation resistant ApoA1. In some cases it may be preferable to administer the oxidation-resistant ApoA1 variant monomers or dimers alone (naked), essentially lipid-free, to treat, ameliorate or prevent the diseases or disorders and conditions described herein. When specific formulations comprising monomers or dimers of oxidation-resistant ApoA1 variant polypeptides, or lipid complexed monomers or dimers of oxidation-resistant ApoA1 variant polypeptides the formulation can be administered to a subject prophylactically, or as a means to ameliorate, treat or diminish the risk of recurrence of a variety of diseases, disorders and conditions associated thereto, for example, Alzheimer's disease, cancer, for example, prostate cancer, breast cancer or colon cancer, cardiovascular diseases, diabetic nephropathy, diabetic retinopathy, disorders of glucose metabolism, dyslipidemia, dyslipoproteinemia, hypertension, impotence, inflammation, rheumatoid arthritis, insulin resistance disorders, lipid elimination in bile, obesity, oxysterol elimination in bile, pancreatitis (pancreatitus) Parkinson's disease, peroxisome proliferator activated receptor (PPAR)-associated disorders, phospholipid elimination in bile, renal diseases, septicemia, metabolic syndrome disorders, thrombotic disorders, C-reactive protein imbalance and insufficient bile production. In some embodiments, the diseases, disorders and conditions associated thereto treatable with the pharmaceutical compositions herein can relate to dyslipidemia related diseases. As used herein, the terms “dyslipidemia” or “dyslipidemic” refer to an abnormally elevated or decreased level of lipid in the blood plasma, including, but not limited to, the altered level of lipid associated with the following conditions: coronary heart disease; coronary artery disease; cardiovascular disease, hypertension, restenosis, vascular or perivascular diseases; dyslipidemic disorders; dyslipoproteinemia; high levels of low density lipoprotein cholesterol; high levels of very low density lipoprotein cholesterol; low levels of high density lipoproteins; high levels of lipoprotein Lp(a) cholesterol; high levels of apolipoprotein B; atherosclerosis (including treatment and prevention of atherosclerosis); hyperlipidemia; hypercholesterolemia; familial hypercholesterolemia (FH); familial combined hyperlipidemia (FCH); lipoprotein lipase deficiencies, such as hypertriglyceridemia, hypoalphalipoproteinemia, and hypercholesterolemialipoprotein. In other embodiments, specific formulations comprising lipid complexed monomers of oxidation resistant ApoA1 can be used to treat the diseases and disorders provided above.

In some embodiments, the monomers or dimers of oxidation-resistant ApoA1 variant polypeptides, or lipid complexed monomers or dimers of oxidation-resistant ApoA1 variant polypeptides of the present invention are administered to subjects in a biologically compatible form suitable for pharmaceutical administration in vivo to effect cholesterol efflux from lipid containing cells, and/or to reduce the proinflammatory effects of immunomodulators that may be involved in cardiovascular disease. In certain embodiments, the lipid complexed monomers of oxidation resistant ApoA1 of the present invention are administered to subjects in a biologically compatible form suitable for pharmaceutical administration in vivo to effect cholesterol efflux from lipid containing cells, and/or to reduce the proinflammatory effects of immunomodulators that may be involved in cardiovascular disease. By “biologically compatible form suitable for administration in vivo” is meant a form of the monomers or dimers of oxidation-resistant ApoA1 variant polypeptides, or lipid complexed monomers or dimers of oxidation-resistant ApoA1 variant polypeptides to be administered in which any toxic effects are outweighed by the therapeutic effects of the protein. In some embodiments, “biologically compatible form suitable for administration in vivo” is meant a form of the lipid complexed monomers of oxidation resistant ApoA1 to be administered in which any toxic effects are outweighed by the therapeutic effects of the protein. The term “subject” or “patient” is intended to include living organisms in which a therapeutic response can be initiated with administration of the monomers or dimers of oxidation-resistant ApoA1 variant polypeptides, or lipid complexed monomers or dimers of oxidation-resistant ApoA1 variant polypeptides of the present invention, e.g., mammals. The term “subject” can include, mammals, for example, humans, primates, monkeys, dogs, cats, mice, rats, and transgenic species thereof. In some embodiments, the administration to the subject or patient comprises administering the lipid complexed monomers of oxidation resistant ApoA1.

Administration of an agent as described herein can be in any pharmacological form including a therapeutically active amount of an agent alone or in combination with a pharmaceutically acceptable excipient, carrier or vehicle. As used herein, administration of a “therapeutically active” amount of the pharmaceutical compositions and formulations of the present invention is defined as an amount of a monomer or dimer of an oxidation-resistant ApoA1 variant polypeptide, or lipid complexed monomer or dimer of an oxidation-resistant ApoA1 variant polypeptide used in dosages and for periods of time necessary to achieve the desired result, for example, to treat, ameliorate or alleviate to some extent one or more of the symptoms associated with the disease or disorder for which the administration was intended to be treated. In still further embodiments, the therapeutically active amount of the pharmaceutical compositions and formulations of the present invention is the amount of lipid complexed monomer of an oxidation resistant ApoA1 which is used in dosages and for periods of time necessary to achieve the desired result, for example, to treat, ameliorate or alleviate to some extent one or more of the symptoms associated with the disease or disorder for which the administration was intended to be treated. For example, a therapeutically effective amount of monomers or dimers of oxidation-resistant ApoA1 variant polypeptides, or lipid complexed monomers or dimers of oxidation-resistant ApoA1 variant polypeptides, may vary according to factors such as the disease state, age, sex, and weight of the subject, and the ability of the oxidation-resistant ApoA1 variant dimer being administered to a subject in need thereof to elicit a desired response in the subject. In other embodiments, a therapeutically effective amount of lipid complexed monomers of oxidation resistant ApoA1, may vary according to factors such as the disease state, age, sex, and weight of the subject, and the ability of the oxidation-resistant ApoA1 variant dimer being administered to a subject in need thereof to elicit a desired response in the subject. Dosage regimes may be adjusted to provide the optimum therapeutic response. Doses may be titrated to achieve the optimal therapeutic index which takes into account the therapeutic benefit and the existence of deleterious side-effects, such as allergies to the administered pharmaceutical composition, development of antibodies against the monomers or dimers of oxidation-resistant ApoA1 variant polypeptides, or lipid complexed monomers or dimers of oxidation-resistant ApoA1 variant polypeptides. In some embodiments, doses may be titrated to achieve the optimal therapeutic index which takes into account the therapeutic benefit and the existence of deleterious side-effects, such as allergies to the administered pharmaceutical composition, development of antibodies against lipid complexed monomers of oxidation resistant ApoA1. In other embodiments, doses may be titrated to achieve the optimal therapeutic index which takes into account the therapeutic benefit and the existence of deleterious side-effects, such as allergies to the administered pharmaceutical composition, development of antibodies against the lipid complexed monomers of oxidation resistant ApoA1 individually. For example, several divided doses may be administered daily, weekly, and monthly or the dose may be proportionally reduced as indicated by the exigencies of the therapeutic situation.

The monomers or dimers of oxidation-resistant ApoA1 variant polypeptides, or lipid complexed monomers or dimers of oxidation-resistant ApoA1 variant polypeptides or pharmaceutical formulations thereof can be administered by any suitable route known to those of skill in the art that ensures bioavailability in the circulation. In some embodiments, the lipid complexed monomers of oxidation resistant ApoA1 or pharmaceutical formulations thereof can be administered by any suitable route known to those of skill in the art that ensures bioavailability in the circulation. Any route of administration that provides a therapeutically effective amount of the compositions or formulations of the invention can be used. The route of administration can be indicated by the type of pharmaceutical formulation. For example, injectable formulations can be administered parenterally, including, but not limited to, intravenous (IV), intramuscular (IM), intradermal (ID), subcutaneous (SC), intracoronary (IC), intraarterially, pericardially, intraarticular, transdermal, and intraperitoneal (IP) injections. (See, e.g., Robinson et al., 1989, In: Pharmacotherapy: A Pathophysiologic Approach, Ch. 2, pp. 15-34, incorporated herein by reference in its entirety.)

In one embodiment, the monomers or dimers of oxidation-resistant ApoA1 variant polypeptides, or lipid complexed monomers or dimers of oxidation-resistant ApoA1 variant polypeptides or pharmaceutical formulations thereof can be administered parenterally. In some embodiments, lipid complexed monomers of oxidation resistant ApoA1 or pharmaceutical formulations thereof can be administered parenterally. In a more preferred embodiment, the parenteral administration is intravenous. An intravenous administration can be as a bolus, for example, administered over about 2-3 minutes or by continuous infusion, for example, by means of a pump over about 0.5-2 hours or continuously infused, over about 24 hours. In a preferred embodiment, the infusion can be over about 1 to about 3 hours.

In one embodiment, the monomers or dimers of oxidation-resistant ApoA1 variant polypeptides, or lipid complexed monomers or dimers of oxidation-resistant ApoA1 variant polypeptides or pharmaceutical formulations thereof can be administered by intravenous infusion. In some embodiments, lipid complexed monomers of oxidation resistant ApoA1 or pharmaceutical formulations thereof can be administered by intravenous infusion. Any suitable blood vessel can be used as the infusion site. In some embodiments, the pharmaceutical formulation can be infused into the cephalic or median cubital vessel at the antecubital fossa in the arm of a subject. Administration can be either rapid as by injection or over a period of time as by slow infusion or administration of slow release formulation. For treating tissues in the vasculature, administration can be by injection or infusion into regions in which an atherosclerotic plaque exists or may form. Similarly, the monomers or dimers of oxidation-resistant ApoA1 variant polypeptides, or lipid complexed monomers or dimers of oxidation-resistant ApoA1 variant polypeptides of the present invention may be delivered prophylactically to prevent plaque formation in subjects with high total cholesterol or prevent recurrence of cardiovascular morbidity, for example, after a myocardial infarction or an episode of thrombosis in which a plaque has ruptured. In some embodiments, lipid complexed monomers of oxidation resistant ApoA1 of the present invention may be delivered prophylactically to prevent plaque formation in subjects with high total cholesterol or prevent recurrence of cardiovascular morbidity, for example, after a myocardial infarction or an episode of thrombosis in which a plaque has ruptured. When it is intended that monomers or dimers of oxidation-resistant ApoA1 variant polypeptides, or lipid complexed monomers or dimers of oxidation-resistant ApoA1 variant polypeptides be administered to blood vessels of the vasculature, administration can be with one or more agents capable of identifying regions of stenosis and atherosclerotic plaque development. In other embodiments, when it is intended that lipid complexed monomers of oxidation resistant ApoA1 be administered to blood vessels of the vasculature, administration can be with one or more agents capable of identifying regions of stenosis and atherosclerotic plaque development.

Dose administration can be repeated depending upon the pharmacokinetic parameters of the dosage formulation and the route of administration used. It is especially advantageous to formulate parenteral compositions in dosage unit form for ease of administration and uniformity of dosage. Dosage unit form as used herein refers to physically discrete units suited as unitary dosages for the mammalian subjects to be treated; each unit containing a predetermined quantity of active compound calculated to produce the desired therapeutic effect in association with the required pharmaceutical carrier. The specification for the dosage unit forms of the invention are dictated by and directly dependent on (a) the unique characteristics of the active compound and the particular therapeutic effect to be achieved, and (b) the limitations inherent in the art of compounding such an active compound for the treatment of sensitivity in individuals. The specific dose can be readily calculated by one of ordinary skill in the art, e.g., according to the approximate body weight or body surface area of the patient or the volume of body space to be occupied. The dose will also be calculated dependent upon the particular route of administration selected. Further refinement of the calculations necessary to determine the appropriate dosage for treatment is routinely made by those of ordinary skill in the art. Such calculations can be made without undue experimentation by one skilled in the art in light of the activity disclosed herein in assay preparations of target cells. Exact dosages are determined in conjunction with standard dose-response studies. It will be understood that the amount of the composition actually administered will be determined by a practitioner, in the light of the relevant circumstances including the condition or conditions to be treated, the choice of composition to be administered, the age, weight, and response of the individual patient, the severity of the patient's symptoms, and the chosen route of administration.

For the monomers or dimers of oxidation-resistant ApoA1 variant polypeptides, or lipid complexed monomers or dimers of oxidation-resistant ApoA1 variant polypeptides or composition or formulation thereof used in the methods of the invention, the therapeutically effective dose can be estimated initially from cell culture assays. In other embodiments, for lipid complexed monomers of oxidation resistant ApoA1, or composition or formulation thereof used in the methods of the invention, the therapeutically effective dose can be estimated initially from cell culture assays. A dose may be formulated in animal models to achieve a circulating plasma concentration range that includes the IC₅₀ (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography.

In one embodiment of this invention, a monomer or dimer of an oxidation-resistant ApoA1 variant polypeptide, may be therapeutically administered by implanting into patients vectors or cells capable of producing a biologically-active form of the oxidation-resistant ApoA1 variant polypeptide monomer or a precursor of an oxidation-resistant ApoA1 variant polypeptide monomer of the present invention, i.e. a molecule e.g. a polynucleotide that can be readily converted to a biological-active form of oxidation-resistant ApoA1 variant monomer or dimer, for example, by implanting the polynucloeotide of SEQ ID NO:34 in the body, preferably, to be expressed by endothelial cells intimately associated with the lumen of coronary or other blood vessels.

### Combinations of Active Agents

In some embodiments, the monomers or dimers of oxidation-resistant ApoA1 variant polypeptides, or lipid complexed monomers or dimers of oxidation-resistant ApoA1 variant polypeptides, or pharmaceutical formulations thereof can be used alone or in combination therapy with other therapies in the methods of the present invention. In other embodiments, the lipid complexed monomers of oxidation resistant ApoA1 or pharmaceutical formulations thereof can be used alone or in combination therapy with other therapies in the methods of the present invention. Such therapies include, but are not limited to simultaneous or sequential administration of other drugs. In certain embodiments, the methods and pharmaceutical compositions and formulations of the invention can be used in combination with other drugs to achieve the methods provided herein. The co-administration of another drug can be to treat, prevent or ameliorate accompanying diseases, conditions, disorders or symptoms, or to reduce unwanted side-effects, for example, cholesterol reducing statin drugs administered treat a co-existing dyslipidemia or angina. In certain embodiments, the methods provide for co-administration of drugs and or other active agents to treat or prevent pain accompanying acute coronary syndromes.

As described above, conventional therapy for ischemic events, including myocardial infarction, angina and acute coronary syndromes have not targeted the underlying pathology of atherosclerotic plaque formation and plaque rupture. For example, a subject presenting with cardiac chest discomfort or other ischemic symptoms can be prepared for emergent percutaneous coronary intervention (PCI) with anti-clotting or antithrombotic drugs such as aspirin, clopidogrel, heparin, eptifibatide or abciximab. For subjects that have been resuscitated from sudden cardiac death, the administration of amiodarone is recommended. Oxygen is often supplied to the subject, generally by mask or nasal cannula and vital signs closely monitored, including, oxygen saturation via pulse oximetry. For long-term treatment, statins of HMGCoA reductase inhibitor are often administered to the subject to reduce cholesterol levels. However, no established therapy is approved for diminishing cholesterol mediated vessel occlusion due to plaque build-up or the stabilization or reduction of plaque in immunologically inflamed vessel sites.

In some embodiments, monomers or dimers of oxidation-resistant ApoA1 variant polypeptides, or lipid complexed monomers or dimers of oxidation-resistant ApoA1 variant polypeptides or pharmaceutical formulations thereof can be administered with other pharmaceutically active drugs including, but not limited to, alpha/beta adrenergic antagonists, antiadrenergic agents, alpha-1 adrenergic antagonists, beta adrenergic antagonists, AMP kinase activators, angiotensin converting enzyme (ACE) inhibitors, angiotensin 11 receptor antagonists, calcium channel blockers, antiarrhythmic agents, vasodilators, nitrates, vasopressors, inotropic agents, diuretics, anticoagulation agents, antiplatelet aggregation agents, thrombolytic agents, antidiabetic agents, antioxidants, anti-inflammatory agents, bile acid sequestrants, statins, cholesterol ester transfer protein (CETP) inhibitors, cholesterol reducing agents/lipid regulators, drugs that block arachidonic acid conversion, estrogen replacement therapy, fatty acid analogues, fatty acid synthesis inhibitors, fibrates, histidine, nicotinic acid derivatives, peroxisome proliferator activator receptor agonists or antagonists, fatty acid oxidation inhibitors, thalidomide or thiazolidinediones (Drug Facts and Comparisons, updated monthly, January 2003, Wolters Kluwer Company, St. Louis, Mo.; Physicians Desk Reference (56.sup.th edition, 2002) Medical Economics). In other embodiments, the lipid complexed monomers of oxidation resistant ApoA1 or pharmaceutical formulations thereof, can be administered with pharmaceutically active drugs described above.

Other drugs singly or in combination, that can add to or can synergize the beneficial properties of the monomers or dimers of oxidation-resistant ApoA1 variant polypeptides, or lipid complexed monomers or dimers of oxidation-resistant ApoA1 variant polypeptides or pharmaceutical formulations thereof include but are not limited to: Alpha/Beta Adrenergic Antagonists (“β-blockers”) such as, carvediol, (Coreg®); labetalol HCl, (Nonmodyne®); Antiadrenergic Agents such as guanadrel, (Hylorel®); guanethidine, (Ismelin®); reserpine, clonidine, (Catapres® and Catapres-TTS®); guanfacine, (Tenex®); guanabenz, (Wytensin®); methyldopa and methyldopate, (Aldomet®); Alpha-1 Adrenergic Antagonist such as doxazosin, (Cardura®); prazosin, (Minipress®); terazosin, (Hytrin®); and phentolamine, (Regitine®); Beta Andrenergic Antagonists such as sotalol, (Betapace AF® and Betapace®); timolol, (Blocadren®); propranolol, (InderalLA® and Inderal®); betaxolol, (Kerlone®); acebutolol, (Sectral®); atenolol, (Tenormin®); metoprolol, (Lopressor® and Toprol-XL®); bisoprolol, (Zebata®); carteolol, (Cartrol®); esmolol, (Brevibloc®); naldolol, (Corgard®); penbutolol, (Levatol®); and pindolol, (Visken®); AMP kinase activators such as ESP 31015, (ETC-1001); ESP 31084, ESP 31085, ESP 15228, ESP 55016 and ESP 24232; gemcabene (PD 72953 and CI-1027); and MEDICA 16; Angiotensin Converting Enzyme (ACE) Inhibitors such as quinapril, (Accupril®); benazepril, (Lotensin®); captopril, (Capoten®); enalapril, (Vasotec®); ramipril, (Altace®); fosinopril (Monopril®); moexipril, (Univasc®); lisinopril, (Prinivil® and Zestril®); trandolapril, (Mavik®), perindopril, (Aceon®); and Angiotension II Receptor Antagonists such as candesaartan, (Atacand®); irbesartan, (Avapro®); losartan, (Cozaar®); valsartan, (Diovan®); telmisartan, (Micardis®); eprosartan, (Tevetan®); and olmesartan, (Benicar®); Calcium Channel Blockers such as nifedipine, (Adalat®, Adalat CC®, Procardia® and Procardia XL®)); verapamil, (Calan®, CalanSR®, Covera-HS®, IsoptinSR®, Verelan® and VerelanPM®); diltiazem, (Cardizem®, CardizemCD® and Tiazac®); nimodipine, (Nimotop®); amlodipine, (Norvasc®); felodipine, (Plendil®); nisoldipine, (Sular®); bepridil, (Vascor®); isradipine, (DynaCirc®); and nicardipine, (Cardene®); Antiarrhythmics such as various quinidines; procainamide, (Pronestyl® and Procan®); lidocaine, (Xylocalne®); mexilitine, (Mexitil®); tocamide, (Tonocard®); flecamide, (Tambocor®); propafenone (Rythmol®), moricizine, (Ethmozine®); ibutilide, (Covert®); disopyramide, (Norpace®); bretylium, (Bretylol®); amiodarone, (Cordarone®); adenosine, (Adenocard®); dofetilide (Tikosyn®); and digoxin, (Lanoxin®); Vasodilators such as diazoxide, (Hyperstat IV®); hydralazine, (Apresoline®); fenoldopam, (Corolpam®); minoxidil, (Loniten®); and nitroprusside, (Nipride®); Nitrates such as isosorbide dinitrate; (Isordil® and Sorbitrate®); isosorbide mononitrate, (Imdur®, Ismo® and Monoket®); Nitroglycerin paste, (Nitrol®); various nitroglycerin patches; nitroglycerin SL, (Nitrostat®), Nitrolingual spray; and nitroglycerin IV, (Tridil®); Vassopressors such as norepinephrine, (Levophed®); and phenylephrine, (Neo-Synephrine®); Inotrophic Agents such as aminone; (Inocor®); dopamine, (Intropine®); dobutamine, (Dobutrex®); epinephrine, (Adrenalin®); isoproternol, (Isuprel®), milrinone, (Primacor®); Diuretics such as spironolactone, (Aldactone®); torsemide, (Demadex®); hydroflumethiazide, (Diucardin®); chlorothiazide, (Diuril®); ethacrynic acid, (Edecrin®); hydrochlorothiazide, (hydroDIURIL® and Microzide®); amiloride, (Midamor®); chlorthalidone, (Thalitone® and Hygroton®); bumetanide, (Bumex®); furosemide, (Lasix®); indapamide, (Lozol®); metolazone, (Zaroxolyn®); triamterene, (Dyrenium®); and combinations of triamterene and hydrochlorothiazide (Dyazide® and Maxzide®); Antithrombotics/Anticoagulants/Antiplatelet such as bivalirudin, (Angiomax®); lepirudin, (Refludan®); various heparins; danaparoid, (Ogaran®); various low molecular weight heparins; dalteparin, (Fragamin®) enoxaparin, (Lovenox®); tinzaparin, (Innohep®); warfarin, (Coumadin®); dicumarol, (Dicoumarol®); anisindione, (Miradone®); aspirin; argatroban, (Argatroban®); abciximab, (Reopro®); eptifibatide, (Integrilin®); tirofiban, (Aggrastat®); clopidogrel, (Plavix®); ticlopidine, (Ticlid®); and dipyridamole, (Persantine®); Thrombolytics such as alteplase, (Activase®); tissue plasminogen activator (TPA), (Activase®); anistreplase, APSAC, (Eminase®); reteplase, rPA, (Retavasae®); steptokinase, SK, (Streptase®); urokinase, (Abbokinase®); Antidiabetic agents such as metformin, (Glucophage®); glipizide, (Glucotrol®); chlorpropamide, (Diabinese®); acetohexamide, (Dymelor®); tolazamide, (Tolinase®); glimepride, (Amaryl®); glyburide, (DiaBeta® and Micronase®); acarbose, (Precose®); miglitol, (Glyset®); repaflinide, (Prandin®); nateglinide, (Starlix®); rosiglitazone, (Avandia®); and pioglitazone (Actos®); Antioxidants and anti-inflammatory agents; Bile Acid Sequestrants such as cholestyramine, (LoCholest®, Prevalite® and Questran®); colestipol, (Colestid®); and colesevelam, (Welchol®); Statins (drugs that inhibit HMGCoA reductase) such as rovastatin, (Crestor®); fluvastatin, (Lescol®); atorvastatin, (Lipitor®); lovastatin, (Mevacor®); pravastatin, (Pravachol®); and simvastatin, (Zocor®); CETP inhibitors; drugs that block arachidonic acid conversion: Estrogen replacement therapy; Fatty acid analogues such as PD 72953, MEDICA 16, ESP 24232, and ESP 31015; Fatty acid synthesis inhibitors; fatty acid synthesis inhibitors; fatty acid oxidation inhibitors, ranolazine, (Ranexa®); Fibrates such as clofibrate, (Atromid-S®); gemfibrozil, (Lopid®); micronized fenofibrate capsules, (Tricor®); bezafibrate and ciprofibrate; histidine; Nicotinic Acid derivatives such as niacin extended-release tablets, (Niaspan®); Peroxisome proliferator activator receptor agonists and antagonists; thalidomide, (Thalomid®) and compounds described in U.S. Pat. Nos. 6,459,003, 6,506,799 and U.S. Application Publication Nos. 20030022865, 20030018013, 20020077316, and 20030078239 the contents of which are incorporated herein by reference in their entireties. In other embodiments, these drugs can add to or can synergize with lipid complexed monomers of oxidation resistant ApoA1.

When co-administered with other agents, e.g., when co-administered with active agents described above, an “effective amount” of the second or additional agent will depend on the type of drug used. Suitable dosages are known for approved agents and can be adjusted by the skilled artisan according to the condition of the subject, the type of condition(s) being treated and the amount of a compound described herein being used. In cases where no amount is expressly noted, an effective amount should be assumed. For example, pharmaceutical compositions of monomers or dimers of oxidation-resistant ApoA1 variant polypeptides, or lipid complexed monomers or dimers of oxidation-resistant ApoA1 variant polypeptides, described herein can be administered to a subject in need thereof in a therapeutically effective dosage range (i.e. amount of the oxidation-resistant ApoA1 variant dimer or monomer) from between about 0.01 to about 100 mg/kg of body weight/day, from about 0.01 to about 50 mg/kg body weight/day, from about 0.01 to about 30 mg/kg body weight/day, from about 0.01 to about 10 mg/kg body weight/day, and preferably, from about 0.5 mg/kg to about 5 mg/kg body weight. In still other embodiments, pharmaceutical compositions of lipid complexed monomers of oxidation resistant ApoA1 described herein can be administered to a subject in need thereof in a therapeutically effective dosage range (i.e. amount of the oxidation-resistant ApoA1 variant dimer or monomer) from between about 0.01 to about 100 mg/kg of body weight/day, from about 0.01 to about 50 mg/kg body weight/day, from about 0.01 to about 30 mg/kg body weight/day, from about 0.01 to about 10 mg/kg body weight/day, and preferably, from about 0.5 mg/kg to about 5 mg/kg body weight. For therapeutically effective compositions, the dose of the monomers or dimers of oxidation-resistant ApoA1 variant polypeptides, or lipid complexed monomers or dimers of oxidation-resistant ApoA1 variant polypeptides, for example, a lipid complex comprising POPC and/or DPPC, can range from about 0.01 to about 100 mg/kg body weight/day, from about 0.01 to about 50 mg/kg body weight/day, from about 0.01 to about 30 mg/kg body weight/day, from about 0.01 to about 10 mg/kg body weight/day, (generally, from about 0.5 mg/kg to about 5 mg/kg body weight). In other embodiments, therapeutically effective compositions includes a dose of the lipid complexed monomers of oxidation resistant ApoA1, for example, a lipid complex comprising POPC and/or DPPC, can range from about 0.01 to about 100 mg/kg body weight/day, from about 0.01 to about 50 mg/kg body weight/day, from about 0.01 to about 30 mg/kg body weight/day, from about 0.01 to about 10 mg/kg body weight/day, (generally, from about 0.5 mg/kg to about 5 mg/kg body weight). As used herein, the amount of the dosage refers to the amount of the oxidation-resistant ApoA1 variant monomer or dimer or oxidation-resistant ApoA1 monomer, i.e. the protein, and not of the oxidation-resistant ApoA1 variant monomer or dimer lipid complex or lipid complexed oxidation-resistant ApoA1 monomers administered on a weight per weight of the patient per day basis.

When a “combination therapy” is employed, an effective amount can be achieved using a first amount of a monomer or a dimer of an oxidation-resistant ApoA1 variant polypeptide, or a lipid complexed monomer or a dimer of an oxidation-resistant ApoA1 variant polypeptide, or functional fragments thereof described herein or a pharmaceutically acceptable salt, solvate (e.g., hydrate), thereof and a second amount of an additional suitable therapeutic agent (e.g., an agent to treat an associated comorbidity, e.g. hypertension, arrhythmia, allergy, infection, ischemia, pain, or inflammation of the tissue, organ or blood vessel leading to the diseases and disorders described herein). In other embodiments, when a “combination therapy” is employed, an effective amount can be achieved using a first amount of a lipid complexed monomer of oxidation resistant ApoA1 or functional fragments thereof described herein or a pharmaceutically acceptable salt, solvate (e.g., hydrate), thereof and a second amount of an additional suitable therapeutic agent (e.g., an agent to treat an associated comorbidity, e.g. hypertension, arrhythmia, allergy, infection, ischemia, pain, or inflammation of the tissue, organ or blood vessel leading to the diseases and disorders described herein).

In some embodiments of the present invention, the monomer or a dimer of an oxidation-resistant ApoA1 variant polypeptide, or a lipid complexed monomer or a dimer of an oxidation-resistant ApoA1 variant polypeptide, and, in some embodiments, one or more additional therapeutic agents, are each administered in an effective amount (i.e., each in an amount which would be therapeutically effective if administered alone). In some embodiments of the present invention, the lipid complexed monomer of oxidation resistant ApoA1, and, in some embodiments, one or more additional therapeutic agents, are each administered in an effective amount (i.e., each in an amount which would be therapeutically effective if administered alone). In other embodiments, the monomer or a dimer of an oxidation-resistant ApoA1 variant polypeptide, or a lipid complexed monomer or a dimer of an oxidation-resistant ApoA1 variant polypeptide and optionally, the one or more additional therapeutic agents, are each administered in an amount which alone does not provide a therapeutic effect (a sub-therapeutic dose). In other embodiments, the lipid complexed monomer of oxidation resistant ApoA1 and optionally, the one or more additional therapeutic agents, are each administered in an amount which alone does not provide a therapeutic effect (a sub-therapeutic dose). In yet other embodiments, the monomer or a dimer of an oxidation-resistant ApoA1 variant polypeptide, or a lipid complexed monomer or a dimer of an oxidation-resistant ApoA1 variant polypeptide can be administered in a therapeutically effective amount, while the one or more additional therapeutic agents are administered in a sub-therapeutic dose. In other embodiments, the lipid complexed monomer of oxidation resistant ApoA1 can be administered in a therapeutically effective amount, while the one or more additional therapeutic agents are administered in a sub-therapeutic dose. In still other embodiments, the monomer or a dimer of an oxidation-resistant ApoA1 variant polypeptide, or a lipid complexed monomer or a dimer of an oxidation-resistant ApoA1 variant polypeptide or a lipid complexed monomer of oxidation resistant ApoA1 can be administered in a sub-therapeutic dose, while the one or more additional therapeutic agents, for example, a suitable dyslipidemia or cardiovascular therapeutic agent is administered in a therapeutically effective amount. In still further embodiments, the lipid complexed monomer of oxidation resistant ApoA1 can be administered in a sub-therapeutic dose, while the one or more additional therapeutic agents, for example, a suitable dyslipidemia or cardiovascular therapeutic agent is administered in a therapeutically effective amount.

As used herein, the terms “in combination” or “co-administration” can be used interchangeably to refer to the use of more than one therapy (e.g., one or more prophylactic and/or therapeutic agents). The use of the terms does not restrict the order in which therapies (e.g., prophylactic and/or therapeutic agents) are administered to a subject.

Co-administration encompasses administration of the first and second amounts of the active agents (i.e. a monomer or a dimer of an oxidation-resistant ApoA1 variant polypeptide, or a lipid complexed monomer or a dimer of an oxidation-resistant ApoA1 variant polypeptide, and an additional active agent) in an essentially simultaneous manner, such as in a single pharmaceutical composition, for example, a capsule, tablet or injectable solution having a fixed ratio of first and second amounts, or in multiple, separate capsules solutions for injections or tablets for each. In some embodiments, co-administration encompasses administration of the first and second amounts of the active agents (i.e. a lipid complexed monomer of oxidation resistant ApoA1 and an additional active agent) in an essentially simultaneous manner, such as in a single pharmaceutical composition, for example, a capsule, tablet or injectable solution having a fixed ratio of first and second amounts, or in multiple, separate capsules solutions for injections or tablets for each. In addition, such co-administration also encompasses use of one or more monomers or dimers of an oxidation-resistant ApoA1 variant polypeptide, or a lipid complexed monomer or a dimer of an oxidation-resistant ApoA1 variant polypeptide in a sequential manner in either order. In some embodiments, such co-administration also encompasses use of a lipid complexed monomer of oxidation resistant ApoA1 in a sequential manner in either order. When co-administration involves the separate administration of the first amount of a monomer or a dimer of an oxidation-resistant ApoA1 variant polypeptide, or a lipid complexed monomer or a dimer of an oxidation-resistant ApoA1 variant polypeptide and a second amount of an additional therapeutic agent, the active agents are administered sufficiently close in time to have the desired therapeutic effect. In some embodiments, when co-administration involves the separate administration of the first amount of a lipid complexed monomer of oxidation resistant ApoA1 and a second amount of an additional therapeutic agent, the active agents are administered sufficiently close in time to have the desired therapeutic effect. For example, the period of time between each administration that can result in the desired therapeutic effect, can range from minutes to hours to days and can be determined taking into account the properties of each active agent such as potency, solubility, bioavailability, plasma half-life and kinetic profile. For example, an monomer or a dimer of an oxidation-resistant ApoA1 variant polypeptide, or a lipid complexed monomer or a dimer of an oxidation-resistant ApoA1 variant polypeptide or a lipid complexed monomer of oxidation resistant ApoA1 described herein and a second therapeutic agent can be administered in any order within about 24 hours of each other, within about 16 hours of each other, within about 8 hours of each other, within about 4 hours of each other, within about 1 hour of each other or within about 30 minutes of each other. In other embodiments, a lipid complexed monomer of oxidation resistant ApoA1 described herein and a second therapeutic agent can be administered in any order within about 24 hours of each other, within about 16 hours of each other, within about 8 hours of each other, within about 4 hours of each other, within about 1 hour of each other or within about 30 minutes of each other.

More, specifically, a first therapy (e.g., a prophylactic or therapeutic agent such as a therapy comprising one or more monomers or a dimers of an oxidation-resistant ApoA1 variant polypeptide, or a lipid complexed monomer or a dimer of an oxidation-resistant ApoA1 variant polypeptide or a lipid complexed monomer of oxidation resistant ApoA1 can be administered prior to (e.g., 1 minute, 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks before), concomitantly with, or subsequent to (e.g., 1 minute, 5 minutes, 15 minutes, minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks after) the administration of a second therapy (e.g., a second active agent) to a subject. In still other embodiments, a first therapy (e.g., a prophylactic or therapeutic agent such as a therapy comprising lipid complexed monomers of oxidation resistant ApoA1 can be administered prior to (e.g., 1 minute, 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks before), concomitantly with, or subsequent to (e.g., 1 minute, 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks after) the administration of a second therapy (e.g., a second active agent) to a subject.

### Drug Eluting Stents & Balloon Depots of Drug

In some embodiments, the pharmaceutical compositions of the present invention can be administered to patients in advance or during a surgical procedure for prophylactic effects or to minimize the risk of complications, or to reduce the side effects of the surgical procedure. In some embodiments, a patient can be treated from a few minutes, a few hours, a few days to several weeks before a medical act (e.g., preventive treatment), or during or after a medical act. Administration can be concomitant to or contemporaneous with another invasive therapy, such as, angioplasty, carotid ablation, rotoblader or organ transplant (e.g., heart, kidney, liver, etc.). In one embodiment, intravascular delivery of the pharmaceutical compositions of the present invention can be administered to areas of suspected or known atherosclerosis. Intravascular administration can occur in a number of ways depending on the procedures being performed on the cardiovascularly challenged patient. In one embodiment, a drug eluting stent can be coated, prefilled or connected to depositories containing the present pharmaceutical compositions for administration in situ during or immediately after a catheterization to open a blocked cardiac vessel, for example a coronary artery. The term “stent” is used to refer to small tube used to mechanically open an artery. The stent is collapsed to a small diameter, put over a balloon catheter, inserted through a main artery in the groin (femoral artery) or arm (brachial artery) and threaded up to the narrowed/blocked section of the coronary artery. When it reaches the right location, the balloon is inflated slightly to push any plaque out of the way and to expand the artery (balloon angioplasty). When the balloon is inflated, the stent expands, locks in place and forms a scaffold to hold the artery open. The stent stays in the artery permanently. In certain subjects, a stent reduces the renarrowing that occurs after balloon angioplasty or other procedures that use catheters. A stent also may help restore normal blood flow and keep an artery open if it has been torn or injured by the balloon catheter. Reclosure (restenosis) is a problem with the stent procedure. Drug-eluting stents include stents coated with drugs that are slowly released, but can also include stents which release the oxidation-resistant ApoA1 variant polypeptide monomers or dimers and/or additional active agents of the present invention stored as part of the stent or the stent is operatively connected to a reservoir containing the oxidation-resistant ApoA1 variant polypeptide monomers or dimers of the present invention. In other embodiments, drug-eluting stents include stents coated with drugs that are slowly released, but can also include stents which release a lipid complexed monomer of oxidation resistant ApoA1 and/or additional active agents of the present invention stored as part of the stent or the stent is operatively connected to a reservoir containing the a lipid complexed monomer of oxidation resistant ApoA1 of the present invention. Pharmaceutical compositions of the present invention can be used to help keep a blood vessel from reforming an atherosclerotic plaque, stabilize a preexisting plaque or prevent an existing plaque from rupturing, causing a potential embolism, cerebral or myocardial infarct, or vascular blockage.

### Drug Delivery Systems

In some embodiments, methods for administering a desired drug or medicament in vivo can comprise formulating a lipid complex comprising a drug, for example, a hydrophobic, amphipathic, or hydrophilic cation drug and an oxidation-resistant ApoA1 variant monomer or dimer or oxidation-resistant ApoA1 monomer. In some embodiments, the lipid complex used for the drug delivery can also include ApoA2, for example, human ApoA2 as described herein. In some embodiments of the invention, the lipid complex can be formed by mixing of an amphipathic lipid and a drug of interest in a suitable organic solvent. Chloroform, methylene chloride or methanol are suitable organic solvents, but any highly volatile solvent capable of solubilizing the amphipathic lipid and the drug to be formulated may also be used, providing the solvent has no adverse effects on either the drug or the amphipathic lipid. In an illustrative example, the amphipathic lipid and the drug may be individually solubilized in chloroform and subsequently dried in a sonication vessel, to avoid the need for premixing. If other lipid-soluble components are to be added to the lipid complex these may be added to and dissolved in the lipid/solvent mixture.

Solvent can be removed according to any conventional solvent-removal technique. Solvent evaporation may be effected under reduced pressure for instance, with or without the presence of drying agents or alternatively the solvent may be evaporated under the steady stream of air, nitrogen, or argon. Following solvent removal, the dried lipid mixture is hydrated using an appropriate aqueous buffer, for example, physiological saline or phosphate buffered saline (PBS).

Following addition of the aqueous buffer, an oxidation-resistant ApoA1 variant monomer or dimer or oxidation-resistant ApoA1 monomer of the present invention is added to the mixture and the resulting mixture, is vigorously mixed using an appropriate method, for example by sonication, trituration, or homogenization, to achieve particles of adequately small size. Trituration is known in the art as meaning to pulverize and comminute thoroughly by rubbing or grinding. The particles formed as a result may range in average diameter from about 7 nm to about 25 nm, and are spherical in shape. Size exclusion chromatography can be incorporated to purify particles of a preferred size. The lipid complex containing a hydrophobic drug may be administered in a composition comprising the lipid complex and a pharmaceutically acceptable medium in which the lipid complex are suspended. The preferred route of administration of the composition is systemic, for example, by injection, either intravenously, intramuscularly or subcutaneously or by parenteral infusion. However, the composition may be delivered by other routes, such as topical, interocular, oral, transdermal intranasal or rectal administration.

In one embodiment, the lipid complex can include one or more drugs and lyophilized and packaged into tablets or capsules with or without pharmaceutically acceptable filler materials for oral delivery. Similarly, lipid complexes comprising drugs prepared according to the present invention can be administered through the skin using a patch as is known in the art, or to the lungs via aspiration or spray. The lipid complexes can be suspended in a liquid medium consisting of the aqueous buffer in which the lipid complexes can be formed to synthesize a composition according to an embodiment of the invention. The lipid complexes of a desired size may be isolated from the buffer in which they were formed by size exclusion chromatography, and re-suspended in any pharmaceutically acceptable medium. The composition may be filtered, diluted, or sterilized, as desired. The lipid complexes may be included in a composition comprising a semi-solid medium, for example a cream, if the composition is to be administered topically, or rectally.

Drugs delivered with the lipid complexes according to the invention can be targeted to specific locations in the body. These locations may include individual cells or tissues in specific organs. For example, lipid complexes comprising drugs may be effectively targeted to cells and tissues which contain high levels of ApoA1 receptors, for example but not limited to, cubulin (Kozyraki R. et al., Nat. Med. (1999) 5:656-661) or the scavenger receptor, SR-B1 (Kozarsky K. F. et al., Nature (1997) 387:414-417). Similarly, other formulations comprising additional components which target lipid complexes comprising drugs to other target tissues may be incorporated into the particle carriers. Differently charged particles are taken up at different rates by different tissues.

It is known that the lipid content of a reconstituted HDL particle affects the conformation of the ApoA1 contained therein. The neutral lipid content and the cholesterol ester:triglyceride ratio of a reconstituted HDL particle can effect the stability of the particle. Because the in vitro metabolism of reconstituted HDL is influenced by ApoA1 charge and conformation (J. Biol. Chem. (1996) 271:25145-25151), metabolism of the lipid complexes of the present invention are also effected by lipid composition. Plasma half-life of the lipid complexes and uptake of the drug by particular tissues can be controlled by changing the electrostatic properties of the lipid complex by varying the nature of the charged lipids, for example, charged phospholipid composition present in the lipid complexes. Similarly, due to the amphipathic nature of the present lipid complexes, hydrophilic drugs can be strategically placed in the core of the lipid complex, while hydrophobic drugs can be positioned on the surface of the lipid complex. Without wishing to be bound by theory, the amphipathic, cationic or hydrophilic drug may associate with an the present lipid complex via electrostatic, hydrophobic, covalent interactions, hydrogen bonding, or a combination thereof, or through Van der Waals forces, or a combination of any of the above associations. Certain advantages readily attributable to the lipid complexes of the present invention, comprising oxidation-resistant ApoA1 variant monomer or dimer or oxidation-resistant ApoA1 monomer include increased stability and half-life in the oxidation-resistant form of the ApoA1 variant monomer or dimer, or oxidation-resistant ApoA1 monomer due to resistance to the oxidation effects of myeloperoxidase, NAPDH-oxidase and other oxidant producing enzymes present in the diseased vasculature.

### Methods of Treatment

The pharmaceutical compositions and formulation of the present invention are useful in the treatment of a variety of diseases, disorders and conditions. In a non-limiting fashion, the present dimeric forms of oxidation-resistant ApoA1 polypeptides, lipid complexes containing the oxidation-resistant ApoA1 variant monomers or dimers, and pharmaceutical compositions and formulation based thereon can be used to treat Alzheimer's disease, cancer, cardiovascular disease, diabetic nephropathy, diabetic retinopathy, a disorder of glucose metabolism, dyslipidemia, dyslipoproteinemia, hypertension, impotence, inflammation, rheumatoid arthritis, insulin resistance, lipid elimination in bile, obesity, oxysterol elimination in bile, pancreatitis (pancreatitus) Parkinson's disease, a peroxisome proliferator activated receptor (PPAR)-associated disorder, phospholipid elimination in bile, renal disease, septicemia, metabolic syndrome disorders, thrombotic disorder, C-reactive protein imbalance and insufficient bile production. In other embodiments, the present lipid complexed monomers of oxidation resistant ApoA1 and pharmaceutical compositions and formulation based thereon can be used to treat Alzheimer's disease, cancer, cardiovascular disease, diabetic nephropathy, diabetic retinopathy, a disorder of glucose metabolism, dyslipidemia, dyslipoproteinemia, hypertension, impotence, inflammation, rheumatoid arthritis, insulin resistance, lipid elimination in bile, obesity, oxysterol elimination in bile, pancreatitis (pancreatitus) Parkinson's disease, a peroxisome proliferator activated receptor (PPAR)-associated disorder, phospholipid elimination in bile, renal disease, septicemia, metabolic syndrome disorders, thrombotic disorder, C-reactive protein imbalance and insufficient bile production.

Methods for treating any one or more of: Alzheimer's disease, cancer, cardiovascular disease, diabetic nephropathy, diabetic retinopathy, a disorder of glucose metabolism, dyslipidemia, dyslipoproteinemia, hypertension, impotence, inflammation, rheumatoid arthritis, insulin resistance, lipid elimination in bile, obesity, oxysterol elimination in bile, pancreatitis (pancreatitus) Parkinson's disease, a peroxisome proliferator activated receptor (PPAR)-associated disorder, phospholipid elimination in bile, renal disease, septicemia, metabolic syndrome disorders, thrombotic disorder, C-reactive protein imbalance and insufficient bile production in a subject in need thereof comprises, administering at least one of an oxidation-resistant ApoA1 variant monomer or dimer, a pharmaceutical composition comprising an oxidation-resistant ApoA1 variant monomer or dimer to the subject. In still further embodiments, methods for treating the above diseases and disorders in a subject in need thereof comprises, administering at least one lipid complexed monomer of oxidation resistant ApoA1, or a pharmaceutical composition comprising a lipid complexed monomer of oxidation resistant ApoA1 to the subject.

In some embodiments, the present invention provides a method for treating a disease or disorder associated with dyslipidemia, the method comprising administering to a subject in need of such treatment, a therapeutically effective composition comprising an oxidation-resistant ApoA1 variant monomer or dimer-phospholipid complex or pharmaceutical formulation comprising same. In some embodiments, the present invention provides a method for treating a disease or disorder associated with dyslipidemia, the method comprising administering to a subject in need of such treatment, a therapeutically effective composition comprising a lipid complexed monomer of oxidation resistant ApoA1. In these embodiments, the therapeutically effective composition provides at least one of: (1) improved cholesterol efflux from a biological cell containing cholesterol, (for example a macrophage foam cell) in the presence of at least one oxidant selected from the group of myeloperoxidase (MPO), MPO generated oxidant, an oxidant generated by the MPO/H₂O₂/Cl⁻ system, HOCl/OCl⁻, MPO generated reactive nitrogen species, oxidants generated by the MPO/H₂O₂/NO₂⁻ system, NO₂⁻, ONOO⁻, ONOOCO₂⁻, an oxidant created by the action of ONOO⁻ in the presence of CO₂ or HCO₃⁻ in a buffer, an oxidant created by a glutathione synthase deficiency, a monoaminooxidase deficiency, an oxidant generated by NADPH oxidase, hydrogen peroxide, hypochlorite ion, hypochlorous acid, singlet oxygen, ozone, NO, hydroxyl radical, superoxide anion, peroxyl radical, peroxides of alkali and alkaline earth metals, organic peroxy compounds, peroxy acids, peroxynitriles and mixtures thereof, or (2) the inhibition of a deleterious immune response associated with a coronary or vascular disease (e.g. dyslipidemia) when the oxidation-resistant ApoA1 variant dimer(s) is or are compared to wild-type ApoA1 or naturally occurring ApoA1 variants ApoA1-Milano, or ApoA1-Paris. As used herein, a deleterious immune response can refer to any one of: expression and/or release of a proinflammatory chemokine or cytokine from an immunological cell including but not limited to TNF-α, IL-1β, IL-6, IL-8, IL-12, MIF, MCP-1, CXCL10, lipoprotein uptake by the immunological cell, scavenger receptor A mRNA and protein expression, CD36 expression, CD40/CD40L expression, iNOS expression, expression of dendritic cell markers CD11c, MYOF and FAM62A, superoxide, hydrogen peroxide and myeloperoxidase production and release by an immunological cell, direct macrophage-T-cells interaction and activation, differentiation of macrophage cell type to foam cell type, expression of iPLA2-β by an immunological cell, production of proinflammatory eicosanoids, arachidonic acid, PGE₂, PGD₂, 15-HETE, 12-HETE, and 13-HODE and endothelin-1 expression by an immune cell.

In some embodiments, pharmaceutical compositions of the present invention are also useful in promoting the mobilization and efflux of stored cholesterol located in atherosclerotic plaques and/or sites of inflammation. In a preferred embodiment, the pharmaceutical compositions are used to promote the mobilization and efflux of stored cholesterol from macrophages and other tissues located in atherosclerotic plaques or sites of inflammation in vivo. More preferably, the pharmaceutical compositions are used to promoting the mobilization and efflux of stored cholesterol from macrophages and other tissues located in atherosclerotic plaques or sites of inflammation in mammals and in particular humans.

In some embodiments, methods for determining the efficacy of the oxidation-resistant ApoA1 variant monomers or dimers of the present invention in reverse cholesterol transport (cholesterol efflux from cholesterol or lipid laden cells) are well known. In other embodiments, methods for determining the efficacy of the lipid complexed monomers of oxidation resistant ApoA1 of the present invention in reverse cholesterol transport (cholesterol efflux from cholesterol or lipid laden cells) are well known. Cholesterol efflux can be measured using any known method to those skilled in the art. In one embodiment, cholesterol efflux can be measured using cultured cells, for example, RAW 264.7 murine macrophage cells using methods known in the art.

In some embodiments, a generic method capable of measuring cholesterol efflux in RAW 264.7 murine macrophage cells and other cultured cells can illustratively include the steps: seed cells in complete medium in six-well plates at a density of 2×10⁴ cells/well. After 48 h, the medium is replaced with DMEM supplemented with 5% DCS and 5 μCi/ml [³H]cholesterol is dispersed in 0.1% ethanol (% final volume of media) for 48 h. Before each efflux experiment, cells are washed three times with DMEM and then incubated with DMEM containing HDL3 (50 μg/ml) and 0.2% BSA. Media aliquots are taken at different times of incubation and counted for radioactivity. At the end of the experiment, cells are solubilized in 0.5 N NaOH to determine protein and [³H]cholesterol content. Results determined can be expressed as the percentage of labeled cholesterol remaining in the cells as a function of time.

In some embodiments, a method for treating dyslipidemia or a disease associated with dyslipidemia to a subject in need thereof comprises administering a therapeutically effective dose of an oxidation-resistant ApoA1 variant monomer or dimer, or a phospholipid complex comprising same to the subject. In some embodiments, the method for treating dyslipedemia or a disease associated with dyslipidemia comprises administering a therapeutically effective dose of a lipid complexed monomer of oxidation resistant ApoA1. The method may further include administering an oxidation-resistant ApoA1 variant dimer in an amount from about 0.01 mg/kg of body weight to about 100 mg/kg of body weight of the subject per day, or from about 0.05 mg/kg of body weight to about 75 mg/kg of body weight per day, or from about 0.1 mg/kg of body weight to about 10 mg/kg of body weight of the subject per day.

In some embodiments, the method to treat the diseases and disorders exemplified herein can utilize the oxidation-resistant ApoA1 variant monomer or dimer formulated as an oxidation-resistant ApoA1 variant monomer or dimer: phospholipid complex. In some embodiments, the method to treat the diseases and disorders exemplified herein can utilize a lipid complexed monomer of oxidation resistant ApoA1. In some embodiments, the lipid is a phospholipid, for example, POPC. In some embodiments, the oxidation-resistant ApoA1 variant monomer or dimer: phospholipid complex contains a molar ratio of oxidation-resistant ApoA1 variant dimer to phospholipid ranging from 1 to 1 to 1 to 200. In some embodiments, the monomer of oxidation resistant ApoA1: phospholipid complex contains a molar ratio of the monomer of oxidation resistant ApoA1 to phospholipid ranging from 1 to 1 to 1 to 200. In some embodiments, the method further provides for administration of the oxidation-resistant ApoA1 variant monomer or dimer or phospholipid complexes containing the same via intravenous (IV), intramuscular (IM), intradermal, subcutaneous (SC), intracoronary, intraarterially, pericardially, intraarticular and intraperitoneal (IP) injections or given orally. In other embodiments, the method further provides for administration of the monomer of oxidation resistant ApoA1: phospholipid complex via intravenous (IV), intramuscular (IM), intradermal, subcutaneous (SC), intracoronary, intraarterially, pericardially, intraarticular and intraperitoneal (IP) injections or given orally. In some embodiments, methods for treating dyslipidemia disease comprises inserting a drug eluting stent into the lumen of a blood vessel at or near the site of a plaque or at a site likely to develop a plaque, and delivering a therapeutically effective amount of an oxidation-resistant ApoA1 variant monomer or dimer into the lumen of the blood vessel from the inserted drug eluting stent. In other embodiments, methods for treating dyslipidemia disease comprises inserting a drug eluting stent into the lumen of a blood vessel at or near the site of a plaque or at a site likely to develop a plaque, and delivering a therapeutically effective amount of a lipid complexed monomer of oxidation resistant ApoA1 into the lumen of the blood vessel from the inserted drug eluting stent. In some embodiments, the drug eluting stent delivers a therapeutically effective amount of an oxidation-resistant ApoA1 variant monomer or dimer: phospholipid complex, wherein the molar ratio of the oxidation-resistant ApoA1 variant monomer or dimer to phospholipid ranges from about 1 to 1 to about 1 to 200. In still further embodiments, the drug eluting stent delivers a therapeutically effective amount of a monomer of oxidation resistant ApoA1: phospholipid complex, wherein the molar ratio of the monomer of oxidation resistant ApoA1 to phospholipid ranges from about 1 to 1 to about 1 to 200.

### EXAMPLES

### Example 1

### ApoA1 Variant Polypeptide Modifications

Human purified ApoA1 (hAI) and recombinant wild type ApoA1 (WT AI), WT AI monomer having four tryptophans substituted with phenylalanine (4WF) and oxidation-resistant ApoA1 variant dimer (SEQ ID NO:5) (OxResD) were diluted to the final concentration of 7 μM in PBS (pH 7.2) containing 100 μM EDTA. The reaction mixture was supplemented with purified human myeloperoxidase (MPO) at a concentration of 50 nM (Calbiochem) and HOCl⁻ production was triggered with 100 μM of H₂O₂ (Sigma) to achieve H₂O₂: ApoA1 protein molar ratios 14:1. Oxidative modification of ApoA1 variants was allowed to continue at 37° C. for 60 min at which time reaction was stopped by quenching HOCl⁻ with 2 mmol/L L-methionine. Samples of native and modified proteins were stored at −20° C. until further use.

### Example 2

### Detection of Structural Modifications of ApoA1 Variants

Two g of protein per lane were denatured in SDS-containing sample buffer under reduced (FIG. 3A) vs. non-reducing conditions (FIG. 3B). Samples were electrophoresed on a NuPAGE 4-12% Bis-Tris gel with MOPS running buffer (Invitrogen). Proteins were transferred to a polyvinylidene fluoride (PVDF) membrane and probed sequentially with biotinylated goat anti-human ApoA1 primary antibody (1:1000 dilution, R&D Systems), streptavidin-peroxidase polymer (1:1000 dilution, Sigma), and ApoA1 variants were visualized with an enhanced chemiluminescent substrate (GE Healthcare).

### Example 3

### ATP-Binding Cassette Transporter A1 (ABCA1)-Dependent Cholesterol Efflux

RAW 264.7 murine macrophage cells were labeled with 1% FBS containing 0.3 μCi/mL [3H]-cholesterol and treated with 0.3 mmol/L 8Br-cAMP to induce ABCA1 activity. The cells were washed and chased for 4 hours in serum-free medium containing 5 g/mL of the variously modified vs. non-modified ApoA1 preparations. The radioactivity in the chase media was determined after brief centrifugation to pellet debris. Radioactivity in wells designated “Total” was determined by aspirating the media from wells prior the chase period followed by cell extraction in hexane:isopropanol (3:2) with the solvent evaporated in a scintillation vial prior to counting. The percent cholesterol efflux was calculated as 100 (medium dpm)/(Total dpm) where “Total” represents [3H]-cholesterol present in the cells prior to the chase period.

### Example 4

### ApoA1 Variant Polypeptide Resistance To Oxidation

MPO-mediated damage to ApoA1 protein and its variants was assessed by Western blotting. As shown on FIG. 3A, samples of reduced, non-modified recombinant WT ApoA1, 4WF and OxResD proteins co-migrated with purified hA-I standard and were detected predominantly as a single bands species at ˜26 kD. Likewise, under non-reduced conditions purified hA-I standard as well as recombinant WT ApoA1 and 4WF all demonstrated very similar migration pattern (FIG. 3B). In contrast, non-reduced OxResD variant was present as a higher (˜55 kD) molecular weight immunoreactive band suggesting the dimeric nature of non-modified protein (FIGS. 3A & 3B). MPO-mediated oxidation of ApoA1 and all of its variants resulted in extensive protein modifications and appearance of multiple higher molecular weight immunoreactive bands at ˜50 kD, ˜70 kD and ˜120 kD indicating extensive inter- and intra-molecular cross-linking followed by the formation of protein dimers, trimers and multimers (FIG. 3A).

Cellular lipid efflux to ApoA1, as well as HDL biogenesis, is mediated by the ABC transporters, including ABCA1. Recent in vitro and in vivo studies have shown that ApoA1 can stimulate the removal of preloaded cholesterol from macrophages. ApoA1 in plasma and in atherosclerotic lesions has also been shown to be a selective target for MPO modification. MPO modification of ApoA1 results in decreased cholesterol acceptor and lipid binding activities of ApoA1. To determine whether tryptophan to phenylalanine substitution in 4WF and OxResD variants will afford advantage to their lipid efflux capacity after MPO-mediated damage, in vitro reverse cholesterol transport (RCT) studies were performed using the RAW 264.7 murine macrophage cell line.

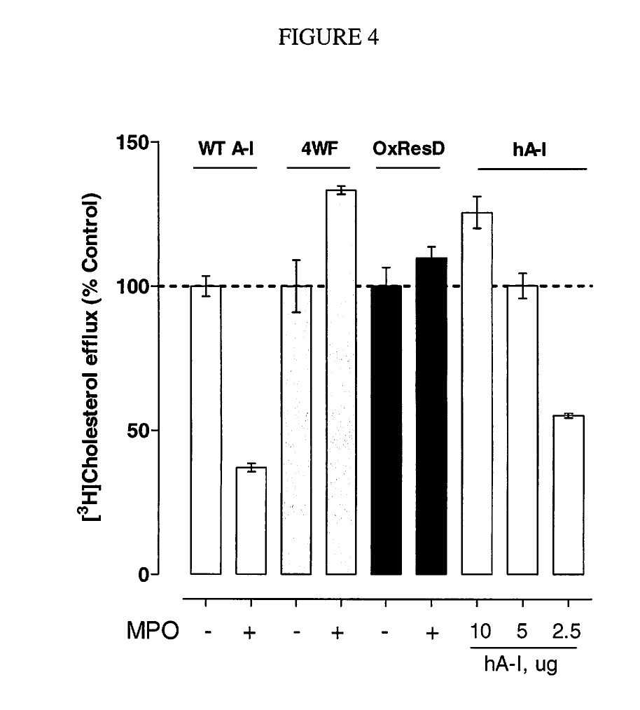

As expected, the ABCA-1-dependent cholesterol efflux ability of the recombinant ApoA1 was markedly reduced by 75-80% upon MPO/HOCl-mediated oxidative damage. In sharp contrast, and in spite of the significant structural modifications, both 4WF and OxResD variants of ApoA1 demonstrated preserved ability to efflux intracellular cholesterol in an ABCA1-dependent fashion (FIG. 4).

The embodiments and the examples described herein are exemplary and not intended to be limiting in describing the full scope of compositions and methods of the present invention. Equivalent changes, modifications and variations of some embodiments, materials, compositions and methods can be made within the scope of the present invention, with substantially similar results.

### EMBODIMENTS

An isolated oxidation-resistant ApoA1 variant dimer comprising a first oxidation-resistant ApoA1 variant polypeptide monomer and a second oxidation-resistant ApoA1 variant polypeptide monomer, at least one of said first and said second monomers comprises at least one amino acid substitution of a tryptophan residue for an oxidation resistant amino acid, or a functional fragment or variant thereof.

The isolated oxidation-resistant ApoA1 variant dimer of embodiments 1, wherein said first and said second monomers are the same.

The isolated oxidation-resistant ApoA1 variant dimer of embodiments 2, wherein said monomers comprise an amino acid sequence of SEQ ID NO:5.

The isolated oxidation-resistant ApoA1 variant dimer of embodiments 2, wherein said monomers comprise an amino acid sequence of SEQ ID NO:6-33.

The isolated oxidation-resistant ApoA1 variant dimer of embodiments 1, wherein each of said first and said second monomers comprise an amino acid sequence of SEQ ID NO:6-33.

The isolated oxidation-resistant ApoA1 variant dimer of embodiments 1, wherein said first monomer comprises an amino acid sequence of SEQ ID NOs:6-33 and said second monomer comprises an amino acid sequence of SEQ ID NO:35 and/or 36.

The isolated oxidation-resistant ApoA1 variant dimer of embodiments 6, wherein said second monomer comprises at least one amino acid substitution of a tryptophan residue for an oxidation resistant amino acid.

The isolated oxidation-resistant ApoA1 variant dimer of embodiments 6, wherein said second monomer further comprises a substitution of an arginine residue for a cysteine.

The isolated oxidation-resistant ApoA1 variant dimer of embodiments 6, wherein said second monomer and said first monomer are linked with a peptide linker.

The isolated oxidation-resistant ApoA1 variant dimer of embodiments 1, wherein said first and said second monomers comprise an amino acid sequence having at least 70% sequence identity to SEQ ID NOs:3-33 or a functional fragment or variant thereof.

The isolated oxidation-resistant ApoA1 variant dimer of embodiments 1, wherein said first and said second monomers comprise an amino acid sequence having at least 80% sequence identity to SEQ ID NOs:3-33 or a functional fragment or variant thereof.

The isolated oxidation-resistant ApoA1 variant dimer of embodiments 1, wherein said first and said second monomers comprise an amino acid sequence having at least 90% sequence identity to SEQ ID NOs:3-33 or a functional fragment or variant thereof.

The isolated oxidation-resistant ApoA1 variant dimer of embodiments 1, wherein said first and said second monomers comprise an amino acid sequence having at least 95% sequence identity to SEQ ID NOs:3-33 or a functional fragment or variant thereof.

The isolated oxidation-resistant ApoA1 variant dimer of embodiments 1, wherein said first and said second monomers comprise an amino acid sequence having at least 98% sequence identity to SEQ ID NOs:3-33 or a functional fragment or variant thereof.

The isolated oxidation-resistant ApoA1 variant dimer of embodiments 1, wherein said first and said second monomers comprise an amino acid sequence having at least 99% sequence identity to SEQ ID NOs:3-33 or a functional fragment or variant thereof.

The isolated oxidation-resistant ApoA1 variant dimer of any one of embodiments 1-15, said dimer further comprising a label operatively attached to at least one of said first and said second monomers of said dimer.

The isolated oxidation-resistant ApoA1 variant dimer of any one of embodiments 1-16, further comprising a radiolabel affixed to at least one of said first and said second monomers of said dimer.

The isolated oxidation-resistant ApoA1 variant dimer of any one of embodiments 1-17, said dimer further comprising a conjugated polyethylene glycol polymer.

The isolated oxidation-resistant ApoA1 variant dimer of embodiments 18, wherein the conjugated polyethylene glycol dimer comprises a mono-conjugated polyethylene glycol polymer.

An oxidation-resistant ApoA1-lipid complex comprising an oxidation-resistant ApoA1 variant dimer, an oxidation resistant ApoA1 variant monomer, or an oxidation resistant ApoA1 monomer and a lipid.

The oxidation-resistant ApoA1-lipid complex of embodiments 20, wherein said oxidation-resistant ApoA1-lipid complex comprising an oxidation-resistant ApoA1 variant dimer, said dimer.

The oxidation-resistant ApoA1-lipid complex of embodiments 21, wherein said oxidation-resistant ApoA1 variant dimer comprises a first oxidation-resistant ApoA1 variant polypeptide monomer and a second oxidation-resistant ApoA1 variant polypeptide monomer, at least one of said first and said second monomers comprises at least one amino acid substitution of a tryptophan residue for an oxidation resistant amino acid, or a functional fragment or variant thereof.

The oxidation-resistant ApoA1-lipid complex of embodiments 22, wherein said first and said second monomers are the same.

The oxidation-resistant ApoA1-lipid complex of embodiments 22, wherein said first and said second monomers comprise an amino acid sequence of SEQ ID NO:5.

The oxidation-resistant ApoA1-lipid complex of embodiments 22, wherein said first or said second monomers comprise an amino acid sequence of SEQ ID NO:6-33.

The oxidation-resistant ApoA1-lipid complex of embodiments 22, wherein each of said first and said second monomers comprise an amino acid sequence of SEQ ID NO:6-33.

The oxidation-resistant ApoA1-lipid complex of embodiments 22, wherein said first monomer comprises an amino acid sequence of SEQ ID NOs:6-33 and said second monomer comprises an amino acid sequence of SEQ ID NO:35 and/or 36.

The oxidation-resistant ApoA1-lipid complex of embodiments 27, wherein said second monomer comprises at least one amino acid substitution of a tryptophan residue for an oxidation resistant amino acid.

The oxidation-resistant ApoA1-lipid complex of embodiments 27, wherein said second monomer further comprises a substitution of an arginine residue for a cysteine.

The oxidation-resistant ApoA1-lipid complex of embodiments 27, wherein said second monomer and said first monomer are linked with a peptide linker.

The oxidation-resistant ApoA1-lipid complex of embodiments 22, wherein said first and said second monomers comprise an amino acid sequence having at least 70% sequence identity to SEQ ID NOs:3-33 or a functional fragment or variant thereof.

The oxidation-resistant ApoA1-lipid complex of embodiments 22, wherein said first and said second monomers comprise an amino acid sequence having at least 80% sequence identity to SEQ ID NOs:3-33 or a functional fragment or variant thereof.

The oxidation-resistant ApoA1-lipid complex of embodiments 22, wherein said first and said second monomers comprise an amino acid sequence having at least 90% sequence identity to SEQ ID NOs:3-33 or a functional fragment or variant thereof.

The oxidation-resistant ApoA1-lipid complex of embodiments 22, wherein said first and said second monomers comprise an amino acid sequence having at least 95% sequence identity to SEQ ID NOs:3-33 or a functional fragment or variant thereof.

The oxidation-resistant ApoA1-lipid complex of embodiments 22, wherein said first and said second monomers comprise an amino acid sequence having at least 98% sequence identity to SEQ ID NOs:3-33 or a functional fragment or variant thereof.

The oxidation-resistant ApoA1-lipid complex of embodiments 22, wherein said first and said second monomers comprise an amino acid sequence having at least 99% sequence identity to SEQ ID NOs:3-33 or a functional fragment or variant thereof.

The oxidation-resistant ApoA1-lipid complex of any one of embodiments 20-36, wherein said lipid comprises a phospholipid.

The oxidation-resistant ApoA1-lipid complex of embodiments 37, wherein said phospholipid comprises small alkyl chain phospholipid, phosphatidylcholine, egg phosphatidylcholine, soybean phosphatidylcholine, dipalmitoylphosphatidylcholine, soy phosphatidylglycerol, egg phosphatidylglycerol, distearoylphosphatidylglycerol, dimyristoylphosphatidylcholine, distearoylphosphatidylcholine, dilaurylphosphatidylcholine, 1-myristoyl-2-palmitoylphosphatidylcholine, 1-palmitoyl-2-myristoylphosphatidylcholine, 1-palmitoyl-2-stearoylphosphatidylcholine, 1-stearoyl-2-palmitoylphosphatidylcholine, dioleoylphosphatidylcholine, 1-palmitoyl-2-oleoylphosphatidylcholine, 1-oleoyl-2-palmitylphosphatidylcholine, dioleoylphosphatidylethanolamine, dilauroylphosphatidylglycerol, phosphatidylserine, phosphatidylethanolamine, phosphatidylinositol, phosphatidylglycerol, diphosphatidylglycerol, dimyristoylphosphatidylglycerol, dipalmitoylphosphatidylglycerol, distearoylphosphatidylglycerol, dioleoylphosphatidylglycerol, phosphatidic acid, dimyristoylphosphatidic acid, dipalmitoylphosphatidic acid, dimyristoylphosphatidylethanolamine, dipalmitoylphosphatidylethanolamine, dimyristoylphosphatidylserine, dipalmitoylphosphatidylserine, brain phosphatidylserine, sphingomyelin, sphingolipids, brain sphingomyelin, dipalmitoylsphingomyelin, distearoylsphingomyelin, galactocerebroside, gangliosides, cerebrosides, phosphatidylglycerol, phosphatidic acid, lysolecithin, lysophosphatidylethanolamine, cephalin, cardiolipin, dicetylphosphate, distearoyl-phosphatidylethanolamine and cholesterol and its derivatives.

The oxidation-resistant ApoA1-lipid complex of embodiments 38, wherein said phospholipid complex comprises POPC, DPPC or combinations thereof.

The oxidation-resistant ApoA1-lipid complex of embodiments 39, wherein said phospholipid comprises POPC.

The oxidation-resistant ApoA1-lipid complex of any one of embodiments 20-40, wherein said oxidation-resistant ApoA1-lipid complex comprises a molar ratio of oxidation-resistant ApoA1 to lipid from about 1:1 to about 1:300.

The oxidation-resistant ApoA1-lipid complex of embodiments 41, wherein said oxidation-resistant ApoA1-lipid complex molar ratio is from 1 to 120 to 1 to 130-150.

The oxidation-resistant ApoA1-lipid complex of any one of embodiments 20-42, wherein said oxidation-resistant ApoA1-lipid complex further comprises APOA2, paraoxonase lutein, beta-carotene, lycopene, flavones, flavinoids, hydrophobic agents, or combinations thereof.

A pharmaceutical composition comprising an oxidation-resistant ApoA1 variant dimer according to any one of embodiments 1-19, an oxidation-resistant ApoA1 variant monomer, an oxidation-resistant ApoA1 monomer of SEQ ID NO:35-lipid complex, a lipid complexed oxidation-resistant ApoA1 variant monomer, a lipid complexed oxidation-resistant ApoA1 variant dimer, or combinations thereof and a pharmaceutically acceptable excipient, carrier or vehicle.

The pharmaceutical composition according to embodiments 44, comprising an oxidation-resistant ApoA1 variant dimer according to any one of embodiments 20-43, an oxidation-resistant ApoA1 variant monomer and a pharmaceutically acceptable excipient, carrier or vehicle.

The pharmaceutical composition comprising an ApoA1-lipid complex according to any one of embodiments 20-43 and a pharmaceutically acceptable excipient, carrier or vehicle.

The pharmaceutical composition according to embodiments 45, wherein said ApoA1-lipid complex comprises POPC, DPPC, or combinations thereof.

The pharmaceutical composition according to embodiments 46, wherein said ApoA1-lipid complex comprises POPC.

The pharmaceutical composition of any one of embodiments 45-47, wherein the molar ratio between said oxidation-resistant ApoA1 variant dimer, oxidation resistant ApoA1 variant monomer, or oxidation resistant ApoA1 monomer and said lipid is from about 1:1 to about 1:300.

A method for treating a disease or disorder in a subject in need thereof, the method comprising: administering a therapeutically effective amount of an isolated oxidation-resistant ApoA1 variant dimer, an oxidation-resistant ApoA1 variant monomer, an oxidation-resistant ApoA1 monomer of SEQ ID NO:35-lipid complex, a lipid complexed oxidation-resistant ApoA1 variant monomer, a lipid complexed oxidation-resistant ApoA1 variant dimer, or combinations thereof to the subject.

The method of embodiments 49, wherein administering the therapeutically effective amount of said isolated oxidation-resistant ApoA1 variant dimer, an oxidation-resistant ApoA1 variant monomer, an oxidation-resistant ApoA1 monomer of SEQ ID NO:35-lipid complex, a lipid complexed oxidation-resistant ApoA1 variant monomer, a lipid complexed oxidation-resistant ApoA1 variant dimer orally, parentally, intravenous (IV), intramuscular (IM), intradermal, subcutaneous (SC), intracoronary, transdermally, intraarterially, pericardially, intraarticular and intraperitoneal (IP) injection or combinations thereof.

The method of embodiments 50, wherein the therapeutically effective amount of the oxidation-resistant ApoA1 variant dimer is administered parentally.

The method of embodiments 50, wherein administering said oxidation-resistant ApoA1 variant dimer comprises administering said dimer intravenously.

The method of any one of embodiments 49-52, wherein the disease or disorder comprises: Alzheimer's disease, cancer, for example, prostate cancer, breast cancer or colon cancer, cardiovascular diseases, diabetic nephropathy, diabetic retinopathy, disorders of glucose metabolism, dyslipidemia, dyslipoproteinemia, hypertension, impotence, inflammation, rheumatoid arthritis, insulin resistance disorders, lipid elimination in bile, obesity, oxysterol elimination in bile, pancreatitis (pancreatitus) Parkinson's disease, peroxisome proliferator activated receptor (PPAR)-associated disorders, phospholipid elimination in bile, renal diseases, septicemia, metabolic syndrome disorders, thrombotic disorders, C-reactive protein imbalance and insufficient bile production or combinations thereof.

The method of embodiments 53, wherein the disease or disorder is dyslipidemia or dyslipoproteinemia.

The method of embodiments 54, wherein the dyslipidemia or dyslipoproteinemia disease or disorder comprises: coronary heart disease; coronary artery disease; cardiovascular disease, hypertension, restenosis, vascular or perivascular diseases; dyslipidemic disorders; dyslipoproteinemia; high levels of low density lipoprotein cholesterol; high levels of very low density lipoprotein cholesterol; low levels of high density lipoproteins; high levels of lipoprotein Lp(a) cholesterol; high levels of apolipoprotein B; atherosclerosis (including treatment and prevention of atherosclerosis); hyperlipidemia; hypercholesterolemia; familial hypercholesterolemia (FH); familial combined hyperlipidemia (FCH); lipoprotein lipase deficiencies, hypertriglyceridemia, hypoalphalipoproteinemia, hypercholesterolemialipoprotein, or combinations thereof.

The method of any one of embodiments 49-55, wherein the therapeutically effective amount of the isolated oxidation-resistant ApoA1 variant dimer, an oxidation-resistant ApoA1 variant monomer, an oxidation-resistant ApoA1 monomer of SEQ ID NO:35-lipid complex, administered to a subject is administered in an amount ranging from about 0.01 to about 100 mg/kg body weight/day.

The method of embodiments 56, wherein the therapeutically effective amount of the isolated oxidation-resistant ApoA1 variant dimer, oxidation-resistant ApoA1 variant monomer, or oxidation-resistant ApoA1 monomer of SEQ ID NO:35-lipid complex administered to a subject is administered in an amount ranging from about 0.1 to about 10 mg protein/kg body weight/day.

The method of any one of embodiments 49-57, wherein said isolated oxidation-resistant ApoA1 variant dimer, oxidation-resistant ApoA1 variant monomer, or oxidation-resistant ApoA1 monomer of SEQ ID NO:35-lipid complex is formulated and administered as a phospholipid complex.

The method of any one of embodiments 58, wherein said phospholipid complex comprises POPC, DPPC, or combinations thereof.

The method of embodiments 59, wherein said phospholipid complex comprises POPC.

The method of embodiments 60 wherein said phospholipid complex comprises a molar ratio of isolated oxidation-resistant ApoA1 variant dimer, oxidation-resistant ApoA1 variant monomer, or oxidation-resistant ApoA1 monomer of SEQ ID NO:35 to POPC from about 1:1 to about 1:300.

A method for inducing cholesterol efflux in a cell in the presence of a myleoperoxidase mediated oxidant, said method comprises:

a. providing a lipid containing cell;

b. adding a therapeutically effective amount of an oxidation-resistant ApoA1 variant dimer to said cell in the presence of said myleoperoxidase mediated oxidant; and

c. measuring cholesterol efflux from said cell.

1. An isolated oxidation-resistant ApoA1 variant dimer comprising a first oxidation-resistant ApoA1 variant polypeptide monomer and a second oxidation-resistant ApoA1 variant polypeptide monomer, at least one of said first and said second monomers comprises at least one amino acid substitution of a tryptophan residue for an oxidation resistant amino acid, or a functional fragment or variant thereof.

2. The isolated oxidation-resistant ApoA1 variant dimer of claim 1, wherein said first and said second monomers are the same.

3. The isolated oxidation-resistant ApoA1 variant dimer of claim 2, wherein said monomers comprise an amino acid sequence of SEQ ID NO:5.

4. The isolated oxidation-resistant ApoA1 variant dimer of claim 2, wherein said monomers comprise an amino acid sequence of SEQ ID NO:6-33.

5. The isolated oxidation-resistant ApoA1 variant dimer of claim 1, wherein each of said first and said second monomers comprise an amino acid sequence of SEQ ID NO:6-33.

6. The isolated oxidation-resistant ApoA1 variant dimer of claim 1, wherein said first monomer comprises an amino acid sequence of SEQ ID NOs:6-33 and said second monomer comprises an amino acid sequence of SEQ ID NO:35 or 36.

7. The isolated oxidation-resistant ApoA1 variant dimer of claim 6, wherein said second monomer comprises at least one amino acid substitution of a tryptophan residue for an oxidation resistant amino acid.

8. The isolated oxidation-resistant ApoA1 variant dimer of claim 6, wherein said second monomer further comprises a substitution of an arginine residue for a cysteine.

9. The isolated oxidation-resistant ApoA1 variant dimer of claim 6, wherein said second monomer and said first monomer are linked with a peptide linker.

10. The isolated oxidation-resistant ApoA1 variant dimer of claim 1, wherein said first and said second monomers comprise an amino acid sequence having at least 70% sequence identity to SEQ ID NOs:3-33 or a functional fragment or variant thereof.

11. The isolated oxidation-resistant ApoA1 variant dimer of claim 1, wherein said first and said second monomers comprise an amino acid sequence having at least 80% sequence identity to SEQ ID NOs:3-33 or a functional fragment or variant thereof.

12. The isolated oxidation-resistant ApoA1 variant dimer of claim 1, wherein said first and said second monomers comprise an amino acid sequence having at least 90% sequence identity to SEQ ID NOs:3-33 or a functional fragment or variant thereof.

13. The isolated oxidation-resistant ApoA1 variant dimer of claim 1, wherein said first and said second monomers comprise an amino acid sequence having at least 95% sequence identity to SEQ ID NOs:3-33 or a functional fragment or variant thereof.

14. The isolated oxidation-resistant ApoA1 variant dimer of claim 1, wherein said first and said second monomers comprise an amino acid sequence having at least 98% sequence identity to SEQ ID NOs:3-33 or a functional fragment or variant thereof.

15. The isolated oxidation-resistant ApoA1 variant dimer of claim 1, wherein said first and said second monomers comprise an amino acid sequence having at least 99% sequence identity to SEQ ID NOs:3-33 or a functional fragment or variant thereof.

16. The isolated oxidation-resistant ApoA1 variant dimer of claim 1, said dimer further comprising a label operatively attached to at least one of said first and said second monomers of said dimer.

17. The isolated oxidation-resistant ApoA1 variant dimer of claim 1, said dimer further comprising a radiolabel affixed to at least one of said first and said second monomers of said dimer.

18. The isolated oxidation-resistant ApoA1 variant dimer of claim 1, said dimer further comprising a conjugated polyethylene glycol polymer.

19. The isolated oxidation-resistant ApoA1 variant dimer of claim 18, wherein the conjugated polyethylene glycol dimer comprises a mono-conjugated polyethylene glycol polymer.

20. An oxidation-resistant ApoA1-lipid complex comprising an oxidation-resistant ApoA1 variant dimer, an oxidation resistant ApoA1 variant monomer, or an oxidation resistant ApoA1 monomer and a lipid.

21. The oxidation-resistant ApoA1-lipid complex of claim 20, wherein said oxidation-resistant ApoA1-lipid complex comprising an oxidation-resistant ApoA1 variant dimer, said dimer.

22. The oxidation-resistant ApoA1-lipid complex of claim 21, wherein said oxidation-resistant ApoA1 variant dimer comprises a first oxidation-resistant ApoA1 variant polypeptide monomer and a second oxidation-resistant ApoA1 variant polypeptide monomer, at least one of said first and said second monomers comprises at least one amino acid substitution of a tryptophan residue for an oxidation resistant amino acid, or a functional fragment or variant thereof.

23. The oxidation-resistant ApoA1-lipid complex of claim 22, wherein said first and said second monomers are the same.

24. The oxidation-resistant ApoA1-lipid complex of claim 22, wherein said first and said second monomers comprise an amino acid sequence of SEQ ID NO:5.

25. The oxidation-resistant ApoA1-lipid complex of claim 22, wherein said first or said second monomers comprise an amino acid sequence of SEQ ID NO:6-33.

26. The oxidation-resistant ApoA1-lipid complex of claim 22, wherein each of said first and said second monomers comprise an amino acid sequence of SEQ ID NO:6-33.

27. The oxidation-resistant ApoA1-lipid complex of claim 22, wherein said first monomer comprises an amino acid sequence of SEQ ID NOs:6-33 and said second monomer comprises an amino acid sequence of SEQ ID NO:35 or 36.

28. The oxidation-resistant ApoA1-lipid complex of claim 27, wherein said second monomer comprises at least one amino acid substitution of a tryptophan residue for an oxidation resistant amino acid.

29. The oxidation-resistant ApoA1-lipid complex of claim 27, wherein said second monomer further comprises a substitution of an arginine residue for a cysteine.

30. The oxidation-resistant ApoA1-lipid complex of claim 27, wherein said second monomer and said first monomer are linked with a peptide linker.

31. The oxidation-resistant ApoA1-lipid complex of claim 22, wherein said first and said second monomers comprise an amino acid sequence having at least 70% sequence identity to SEQ ID NOs:3-33 or a functional fragment or variant thereof.

32. The oxidation-resistant ApoA1-lipid complex of claim 22, wherein said first and said second monomers comprise an amino acid sequence having at least 80% sequence identity to SEQ ID NOs:3-33 or a functional fragment or variant thereof.

33. The oxidation-resistant ApoA1-lipid complex of claim 22, wherein said first and said second monomers comprise an amino acid sequence having at least 90% sequence identity to SEQ ID NOs:3-33 or a functional fragment or variant thereof.

34. The oxidation-resistant ApoA1-lipid complex of claim 22, wherein said first and said second monomers comprise an amino acid sequence having at least 95% sequence identity to SEQ ID NOs:3-33 or a functional fragment or variant thereof.

35. The oxidation-resistant ApoA1-lipid complex of claim 22, wherein said first and said second monomers comprise an amino acid sequence having at least 98% sequence identity to SEQ ID NOs:3-33 or a functional fragment or variant thereof.

36. The oxidation-resistant ApoA1-lipid complex of claim 22, wherein said first and said second monomers comprise an amino acid sequence having at least 99% sequence identity to SEQ ID NOs:3-33 or a functional fragment or variant thereof.

37. The oxidation-resistant ApoA1-lipid complex of claim 20, wherein said lipid comprises a phospholipid.

38. The oxidation-resistant ApoA1-lipid complex of claim 37, wherein said phospholipid comprises small alkyl chain phospholipid, phosphatidylcholine, egg phosphatidylcholine, soybean phosphatidylcholine, dipalmitoylphosphatidylcholine, soy phosphatidylglycerol, egg phosphatidylglycerol, distearoylphosphatidylglycerol, dimyristoylphosphatidylcholine, distearoylphosphatidylcholine, dilaurylphosphatidylcholine, 1-myristoyl-2-palmitoylphosphatidylcholine, 1-palmitoyl-2-myristoylphosphatidylcholine, 1-palmitoyl-2-stearoylphosphatidylcholine, 1-stearoyl-2-palmitoylphosphatidylcholine, dioleoylphosphatidylcholine, 1-palmitoyl-2-oleoylphosphatidylcholine, 1-oleoyl-2-palmitylphosphatidylcholine, dioleoylphosphatidylethanolamine, dilauroylphosphatidylglycerol, phosphatidylserine, phosphatidylethanolamine, phosphatidylinositol, phosphatidylglycerol, diphosphatidylglycerol, dimyristoylphosphatidylglycerol, dipalmitoylphosphatidylglycerol, distearoylphosphatidylglycerol, dioleoylphosphatidylglycerol, phosphatidic acid, dimyristoylphosphatidic acid, dipalmitoylphosphatidic acid, dimyristoylphosphatidylethanolamine, dipalmitoylphosphatidylethanolamine, dimyristoylphosphatidylserine, dipalmitoylphosphatidylserine, brain phosphatidylserine, sphingomyelin, sphingolipids, brain sphingomyelin, dipalmitoylsphingomyelin, distearoylsphingomyelin, galactocerebroside, gangliosides, cerebrosides, phosphatidylglycerol, phosphatidic acid, lysolecithin, lysophosphatidylethanolamine, cephalin, cardiolipin, dicetylphosphate, distearoyl-phosphatidylethanolamine and cholesterol and its derivatives.

39. The oxidation-resistant ApoA1-lipid complex of claim 38, wherein said phospholipid complex comprises POPC, DPPC or combinations thereof.

40. The oxidation-resistant ApoA1-lipid complex of claim 39, wherein said phospholipid comprises POPC.

41. The oxidation-resistant ApoA1-lipid complex of claim 20, wherein said oxidation-resistant ApoA1-lipid complex comprises a molar ratio of oxidation-resistant ApoA1 to lipid from about 1:1 to about 1:300.

42. The oxidation-resistant ApoA1-lipid complex of claim 41, wherein said oxidation-resistant ApoA1-lipid complex molar ratio is from 1 to 120 to 1:130-150.

43. The oxidation-resistant ApoA1-lipid complex of claim 20, wherein said oxidation-resistant ApoA1-lipid complex further comprises ApoA2, paraoxonase lutein, beta-carotene, lycopene, flavones, flavinoids, hydrophobic agents, or combinations thereof.

44. A pharmaceutical composition comprising an oxidation-resistant ApoA1 variant dimer according to claim 1, an oxidation-resistant ApoA1 variant monomer, an oxidation-resistant ApoA1 monomer of SEQ ID NO:35-lipid complex, a lipid complexed oxidation-resistant ApoA1 variant monomer, a lipid complexed oxidation-resistant ApoA1 variant dimer, or combinations thereof and a pharmaceutically acceptable excipient, carrier or vehicle.

45. The pharmaceutical composition according to claim 44, comprising an oxidation-resistant ApoA1 variant dimer according to claim 20, an oxidation-resistant ApoA1 variant monomer and a pharmaceutically acceptable excipient, carrier or vehicle.

46. The pharmaceutical composition comprising an ApoA1-lipid complex according to claim 20, and a pharmaceutically acceptable excipient, carrier or vehicle.

47. The pharmaceutical composition according to claim 46, wherein said ApoA1-lipid complex comprises POPC, DPPC, or combinations thereof.

48. The pharmaceutical composition according to claim 47, wherein said ApoA1-lipid complex comprises POPC.

49. The pharmaceutical composition of claim 46, wherein the molar ratio between said oxidation-resistant ApoA1 variant dimer, oxidation resistant ApoA1 variant monomer, or oxidation resistant ApoA1 monomer and said lipid is from about 1:1 to about 1:300.

50. A method for treating a disease or disorder in a subject in need thereof, the method comprising: administering a therapeutically effective amount of an isolated oxidation-resistant ApoA1 variant dimer, an oxidation-resistant ApoA1 variant monomer, an oxidation-resistant ApoA1 monomer of SEQ ID NO:35-lipid complex, a lipid complexed oxidation-resistant ApoA1 variant monomer, a lipid complexed oxidation-resistant ApoA1 variant dimer, or combinations thereof to the subject.

51. The method of claim 50, wherein administering the therapeutically effective amount of said isolated oxidation-resistant ApoA1 variant dimer, an oxidation-resistant ApoA1 variant monomer, an oxidation-resistant ApoA1 monomer of SEQ ID NO:35-lipid complex, a lipid complexed oxidation-resistant ApoA1 variant monomer, a lipid complexed oxidation-resistant ApoA1 variant dimer orally, parentally, intravenous (IV), intramuscular (IM), intradermal, subcutaneous (SC), intracoronary, transdermal, intraarterially, pericardially, intraarticular and intraperitoneal (IP) injection or combinations thereof.

52. The method of claim 51, wherein the therapeutically effective amount of the oxidation-resistant ApoA1 variant dimer is administered parentally.

53. The method of claim 51, wherein administering said oxidation-resistant ApoA1 variant dimer comprises administering said dimer intravenously.

54. The method of claim 50, wherein the disease or disorder comprises: Alzheimer's disease, cancer, for example, prostate cancer, breast cancer or colon cancer, cardiovascular diseases, diabetic nephropathy, diabetic retinopathy, disorders of glucose metabolism, dyslipidemia, dyslipoproteinemia, hypertension, impotence, inflammation, rheumatoid arthritis, insulin resistance disorders, lipid elimination in bile, obesity, oxysterol elimination in bile, pancreatitis (pancreatitus) Parkinson's disease, peroxisome proliferator activated receptor (PPAR)-associated disorders, phospholipid elimination in bile, renal diseases, septicemia, metabolic syndrome disorders, thrombotic disorders, C-reactive protein imbalance and insufficient bile production or combinations thereof.

55. The method of claim 54, wherein the disease or disorder is dyslipidemia or dyslipoproteinemia.

56. The method of claim 55, wherein the dyslipidemia or dyslipoproteinemia disease or disorder comprises: coronary heart disease; coronary artery disease; cardiovascular disease, hypertension, restenosis, vascular or perivascular diseases; dyslipidemic disorders; dyslipoproteinemia; high levels of low density lipoprotein cholesterol; high levels of very low density lipoprotein cholesterol; low levels of high density lipoproteins; high levels of lipoprotein Lp(a) cholesterol; high levels of apolipoprotein B; atherosclerosis (including treatment and prevention of atherosclerosis); hyperlipidemia; hypercholesterolemia; familial hypercholesterolemia (FH); familial combined hyperlipidemia (FCH); lipoprotein lipase deficiencies, hypertriglyceridemia, hypoalphalipoproteinemia, hypercholesterolemialipoprotein, or combinations thereof.

57. The method of claim 50, wherein the therapeutically effective amount of the isolated oxidation-resistant ApoA1 variant dimer, an oxidation-resistant ApoA1 variant monomer, an oxidation-resistant ApoA1 monomer of SEQ ID NO:35-lipid complex, administered to a subject is administered in an amount ranging from about 0.01 to about 100 mg/kg body weight/day.

58. The method of claim 57, wherein the therapeutically effective amount of the isolated oxidation-resistant ApoA1 variant dimer, oxidation-resistant ApoA1 variant monomer, or oxidation-resistant ApoA1 monomer of SEQ ID NO:35-lipid complex administered to a subject is administered in an amount ranging from about 0.1 to about 10 mg protein/kg body weight/day.

59. The method of claim 50, wherein said isolated oxidation-resistant ApoA1 variant dimer, oxidation-resistant ApoA1 variant monomer, or oxidation-resistant ApoA1 monomer of SEQ ID NO:35-lipid complex is formulated and administered as a phospholipid complex.

60. The method of claim 59, wherein said phospholipid complex comprises POPC, DPPC, or combinations thereof.

61. The method of claim 60, wherein said phospholipid complex comprises POPC.

62. The method of claim 61, wherein said phospholipid complex comprises a molar ratio of isolated oxidation-resistant ApoA1 variant dimer, oxidation-resistant ApoA1 variant monomer, or oxidation-resistant ApoA1 monomer of SEQ ID NO:35 to POPC from about 1:1 to about 1:300.

63. A method for inducing cholesterol efflux in a cell in the presence of a myleoperoxidase mediated oxidant, said method comprises:
a. providing a lipid containing cell; and
b. adding a therapeutically effective amount of an oxidation-resistant ApoA1 variant dimer to said cell in the presence of said myleoperoxidase mediated oxidant.


SEQUENCE LISTING (36 sequences)

SEQ ID NO: 1 - protein, 243 aa, Human
DEPPQSPWDRVKDLATVYVDVLKDSGRDYVSQFEGSALGKQLNLKLLDNWDSVTSTFSKLREQLGPVTQEFWDNLEKETEGLRQEMSKDLEEVKAKVQPYLDDFQKKWQEEMELYRQKVEPLRAELQEGARQKLHELQEKLSPLGEEMRDRARAHVDALRTHLAPYSDELRQCLAARLEALKENGGARLAEYHAKATEHLSTLSEKAKPALEDLRQGLLPVLESFKVSFLSALEEYTKKLNTQ

SEQ ID NO: 2 - protein, 243 aa, Human
DEPPQSPWDRVKDLATVYVDVLKDSGRDYVSQFEGSALGKQLNLKLLDNWDSVTSTFSKLREQLGPVTQEFWDNLEKETEGLRQEMSKDLEEVKAKVQPYLDDFQKKWQEEMELYRQKVEPLRAELQEGARQKLHELQEKLSPLGEEMRDCARAHVDALRTHLAPYSDELRQRLAARLEALKENGGARLAEYHAKATEHLSTLSEKAKPALEDLRQGLLPVLESFKVSFLSALEEYTKKLNTQ

SEQ ID NO: 3 - protein, 243 aa, Homo sapiens
  MISC_FEATURE (8)..(8): Xaa can be tryptophan or an oxidant-resistant amino acid
DEPPQSPXDRVKDLATVYVDVLKDSGRDYVSQFEGSALGKQLNLKLLDNXDSVTSTFSKLREQLGPVTQEFXDNLEKETEGLRQEMSKDLEEVKAKVQPYLDDFQKKXQEEMELYRQKVEPLRAELQEGARQKLHELQEKLSPLGEEMRDRARAHVDALRTHLAPYSDELRQCLAARLEALKENGGARLAEYHAKATEHLSTLSEKAKPALEDLRQGLLPVLESFKVSFLSALEEYTKKLNTQ

SEQ ID NO: 4 - protein, 243 aa, Homo sapiens
  MISC_FEATURE (8)..(8): Xaa can be tryptophan or an oxidation-resistant amino acid
DEPPQSPXDRVKDLATVYVDVLKDSGRDYVSQFEGSALGKQLNLKLLDNXDSVTSTFSKLREQLGPVTQEFXDNLEKETEGLRQEMSKDLEEVKAKVQPYLDDFQKKXQEEMELYRQKVEPLRAELQEGARQKLHELQEKLSPLGEEMRDCARAHVDALRTHLAPYSDELRQRLAARLEALKENGGARLAEYHAKATEHLSTLSEKAKPALEDLRQGLLPVLESFKVSFLSALEEYTKKLNTQ

SEQ ID NO: 5 - protein, 243 aa, Human
DEPPQSPFDRVKDLATVYVDVLKDSGRDYVSQFEGSALGKQLNLKLLDNFDSVTSTFSKLREQLGPVTQEFFDNLEKETEGLRQEMSKDLEEVKAKVQPYLDDFQKKFQEEMELYRQKVEPLRAELQEGARQKLHELQEKLSPLGEEMRDRARAHVDALRTHLAPYSDELRQCLAARLEALKENGGARLAEYHAKATEHLSTLSEKAKPALEDLRQGLLPVLESFKVSFLSALEEYTKKLNTQ

SEQ ID NO: 6 - protein, 243 aa, Human
DEPPQSPFDRVKDLATVYVDVLKDSGRDYVSQFEGSALGKQLNLKLLDNWDSVTSTFSKLREQLGPVTQEFWDNLEKETEGLRQEMSKDLEEVKAKVQPYLDDFQKKWQEEMELYRQKVEPLRAELQEGARQKLHELQEKLSPLGEEMRDRARAHVDALRTHLAPYSDELRQCLAARLEALKENGGARLAEYHAKATEHLSTLSEKAKPALEDLRQGLLPVLESFKVSFLSALEEYTKKLNTQ

SEQ ID NO: 7 - protein, 243 aa, Human
DEPPQSPWDRVKDLATVYVDVLKDSGRDYVSQFEGSALGKQLNLKLLDNFDSVTSTFSKLREQLGPVTQEFWDNLEKETEGLRQEMSKDLEEVKAKVQPYLDDFQKKWQEEMELYRQKVEPLRAELQEGARQKLHELQEKLSPLGEEMRDRARAHVDALRTHLAPYSDELRQCLAARLEALKENGGARLAEYHAKATEHLSTLSEKAKPALEDLRQGLLPVLESFKVSFLSALEEYTKKLNTQ

SEQ ID NO: 8 - protein, 243 aa, Human
DEPPQSPWDRVKDLATVYVDVLKDSGRDYVSQFEGSALGKQLNLKLLDNWDSVTSTFSKLREQLGPVTQEFFDNLEKETEGLRQEMSKDLEEVKAKVQPYLDDFQKKWQEEMELYRQKVEPLRAELQEGARQKLHELQEKLSPLGEEMRDRARAHVDALRTHLAPYSDELRQCLAARLEALKENGGARLAEYHAKATEHLSTLSEKAKPALEDLRQGLLPVLESFKVSFLSALEEYTKKLNTQ

SEQ ID NO: 9 - protein, 243 aa, Human
DEPPQSPWDRVKDLATVYVDVLKDSGRDYVSQFEGSALGKQLNLKLLDNWDSVTSTFSKLREQLGPVTQEFWDNLEKETEGLRQEMSKDLEEVKAKVQPYLDDFQKKFQEEMELYRQKVEPLRAELQEGARQKLHELQEKLSPLGEEMRDRARAHVDALRTHLAPYSDELRQCLAARLEALKENGGARLAEYHAKATEHLSTLSEKAKPALEDLRQGLLPVLESFKVSFLSALEEYTKKLNTQ

SEQ ID NO: 10 - protein, 243 aa, Human
DEPPQSPFDRVKDLATVYVDVLKDSGRDYVSQFEGSALGKQLNLKLLDNFDSVTSTFSKLREQLGPVTQEFWDNLEKETEGLRQEMSKDLEEVKAKVQPYLDDFQKKWQEEMELYRQKVEPLRAELQEGARQKLHELQEKLSPLGEEMRDRARAHVDALRTHLAPYSDELRQCLAARLEALKENGGARLAEYHAKATEHLSTLSEKAKPALEDLRQGLLPVLESFKVSFLSALEEYTKKLNTQ

SEQ ID NO: 11 - protein, 243 aa, Human
DEPPQSPFDRVKDLATVYVDVLKDSGRDYVSQFEGSALGKQLNLKLLDNWDSVTSTFSKLREQLGPVTQEFFDNLEKETEGLRQEMSKDLEEVKAKVQPYLDDFQKKWQEEMELYRQKVEPLRAELQEGARQKLHELQEKLSPLGEEMRDRARAHVDALRTHLAPYSDELRQCLAARLEALKENGGARLAEYHAKATEHLSTLSEKAKPALEDLRQGLLPVLESFKVSFLSALEEYTKKLNTQ

SEQ ID NO: 12 - protein, 243 aa, Human
DEPPQSPFDRVKDLATVYVDVLKDSGRDYVSQFEGSALGKQLNLKLLDNWDSVTSTFSKLREQLGPVTQEFWDNLEKETEGLRQEMSKDLEEVKAKVQPYLDDFQKKFQEEMELYRQKVEPLRAELQEGARQKLHELQEKLSPLGEEMRDRARAHVDALRTHLAPYSDELRQCLAARLEALKENGGARLAEYHAKATEHLSTLSEKAKPALEDLRQGLLPVLESFKVSFLSALEEYTKKLNTQ

SEQ ID NO: 13 - protein, 243 aa, Human
DEPPQSPWDRVKDLATVYVDVLKDSGRDYVSQFEGSALGKQLNLKLLDNFDSVTSTFSKLREQLGPVTQEFFDNLEKETEGLRQEMSKDLEEVKAKVQPYLDDFQKKWQEEMELYRQKVEPLRAELQEGARQKLHELQEKLSPLGEEMRDRARAHVDALRTHLAPYSDELRQCLAARLEALKENGGARLAEYHAKATEHLSTLSEKAKPALEDLRQGLLPVLESFKVSFLSALEEYTKKLNTQ

SEQ ID NO: 14 - protein, 243 aa, Human
DEPPQSPWDRVKDLATVYVDVLKDSGRDYVSQFEGSALGKQLNLKLLDNFDSVTSTFSKLREQLGPVTQEFWDNLEKETEGLRQEMSKDLEEVKAKVQPYLDDFQKKFQEEMELYRQKVEPLRAELQEGARQKLHELQEKLSPLGEEMRDRARAHVDALRTHLAPYSDELRQCLAARLEALKENGGARLAEYHAKATEHLSTLSEKAKPALEDLRQGLLPVLESFKVSFLSALEEYTKKLNTQ

SEQ ID NO: 15 - protein, 243 aa, Human
DEPPQSPWDRVKDLATVYVDVLKDSGRDYVSQFEGSALGKQLNLKLLDNWDSVTSTFSKLREQLGPVTQEFFDNLEKETEGLRQEMSKDLEEVKAKVQPYLDDFQKKFQEEMELYRQKVEPLRAELQEGARQKLHELQEKLSPLGEEMRDRARAHVDALRTHLAPYSDELRQCLAARLEALKENGGARLAEYHAKATEHLSTLSEKAKPALEDLRQGLLPVLESFKVSFLSALEEYTKKLNTQ

SEQ ID NO: 16 - protein, 243 aa, Human
DEPPQSPFDRVKDLATVYVDVLKDSGRDYVSQFEGSALGKQLNLKLLDNFDSVTSTFSKLREQLGPVTQEFFDNLEKETEGLRQEMSKDLEEVKAKVQPYLDDFQKKWQEEMELYRQKVEPLRAELQEGARQKLHELQEKLSPLGEEMRDRARAHVDALRTHLAPYSDELRQCLAARLEALKENGGARLAEYHAKATEHLSTLSEKAKPALEDLRQGLLPVLESFKVSFLSALEEYTKKLNTQ

SEQ ID NO: 17 - protein, 243 aa, Human
DEPPQSPFDRVKDLATVYVDVLKDSGRDYVSQFEGSALGKQLNLKLLDNWDSVTSTFSKLREQLGPVTQEFFDNLEKETEGLRQEMSKDLEEVKAKVQPYLDDFQKKFQEEMELYRQKVEPLRAELQEGARQKLHELQEKLSPLGEEMRDRARAHVDALRTHLAPYSDELRQCLAARLEALKENGGARLAEYHAKATEHLSTLSEKAKPALEDLRQGLLPVLESFKVSFLSALEEYTKKLNTQ

SEQ ID NO: 18 - protein, 243 aa, Human
DEPPQSPFDRVKDLATVYVDVLKDSGRDYVSQFEGSALGKQLNLKLLDNFDSVTSTFSKLREQLGPVTQEFWDNLEKETEGLRQEMSKDLEEVKAKVQPYLDDFQKKFQEEMELYRQKVEPLRAELQEGARQKLHELQEKLSPLGEEMRDRARAHVDALRTHLAPYSDELRQCLAARLEALKENGGARLAEYHAKATEHLSTLSEKAKPALEDLRQGLLPVLESFKVSFLSALEEYTKKLNTQ

SEQ ID NO: 19 - protein, 243 aa, Human
DEPPQSPWDRVKDLATVYVDVLKDSGRDYVSQFEGSALGKQLNLKLLDNFDSVTSTFSKLREQLGPVTQEFFDNLEKETEGLRQEMSKDLEEVKAKVQPYLDDFQKKFQEEMELYRQKVEPLRAELQEGARQKLHELQEKLSPLGEEMRDRARAHVDALRTHLAPYSDELRQCLAARLEALKENGGARLAEYHAKATEHLSTLSEKAKPALEDLRQGLLPVLESFKVSFLSALEEYTKKLNTQ

SEQ ID NO: 20 - protein, 243 aa, Human
DEPPQSPFDRVKDLATVYVDVLKDSGRDYVSQFEGSALGKQLNLKLLDNWDSVTSTFSKLREQLGPVTQEFWDNLEKETEGLRQEMSKDLEEVKAKVQPYLDDFQKKWQEEMELYRQKVEPLRAELQEGARQKLHELQEKLSPLGEEMRDCARAHVDALRTHLAPYSDELRQRLAARLEALKENGGARLAEYHAKATEHLSTLSEKAKPALEDLRQGLLPVLESFKVSFLSALEEYTKKLNTQ

SEQ ID NO: 21 - protein, 243 aa, Human
DEPPQSPWDRVKDLATVYVDVLKDSGRDYVSQFEGSALGKQLNLKLLDNFDSVTSTFSKLREQLGPVTQEFWDNLEKETEGLRQEMSKDLEEVKAKVQPYLDDFQKKWQEEMELYRQKVEPLRAELQEGARQKLHELQEKLSPLGEEMRDCARAHVDALRTHLAPYSDELRQRLAARLEALKENGGARLAEYHAKATEHLSTLSEKAKPALEDLRQGLLPVLESFKVSFLSALEEYTKKLNTQ

SEQ ID NO: 22 - protein, 243 aa, Human
DEPPQSPWDRVKDLATVYVDVLKDSGRDYVSQFEGSALGKQLNLKLLDNWDSVTSTFSKLREQLGPVTQEFFDNLEKETEGLRQEMSKDLEEVKAKVQPYLDDFQKKWQEEMELYRQKVEPLRAELQEGARQKLHELQEKLSPLGEEMRDCARAHVDALRTHLAPYSDELRQRLAARLEALKENGGARLAEYHAKATEHLSTLSEKAKPALEDLRQGLLPVLESFKVSFLSALEEYTKKLNTQ

SEQ ID NO: 23 - protein, 243 aa, Human
DEPPQSPWDRVKDLATVYVDVLKDSGRDYVSQFEGSALGKQLNLKLLDNWDSVTSTFSKLREQLGPVTQEFWDNLEKETEGLRQEMSKDLEEVKAKVQPYLDDFQKKFQEEMELYRQKVEPLRAELQEGARQKLHELQEKLSPLGEEMRDCARAHVDALRTHLAPYSDELRQRLAARLEALKENGGARLAEYHAKATEHLSTLSEKAKPALEDLRQGLLPVLESFKVSFLSALEEYTKKLNTQ

SEQ ID NO: 24 - protein, 243 aa, Human
DEPPQSPFDRVKDLATVYVDVLKDSGRDYVSQFEGSALGKQLNLKLLDNFDSVTSTFSKLREQLGPVTQEFWDNLEKETEGLRQEMSKDLEEVKAKVQPYLDDFQKKWQEEMELYRQKVEPLRAELQEGARQKLHELQEKLSPLGEEMRDCARAHVDALRTHLAPYSDELRQRLAARLEALKENGGARLAEYHAKATEHLSTLSEKAKPALEDLRQGLLPVLESFKVSFLSALEEYTKKLNTQ

SEQ ID NO: 25 - protein, 243 aa, Human
DEPPQSPFDRVKDLATVYVDVLKDSGRDYVSQFEGSALGKQLNLKLLDNWDSVTSTFSKLREQLGPVTQEFFDNLEKETEGLRQEMSKDLEEVKAKVQPYLDDFQKKWQEEMELYRQKVEPLRAELQEGARQKLHELQEKLSPLGEEMRDCARAHVDALRTHLAPYSDELRQCLAARLEALKENGGARLAEYHAKATEHLSTLSEKAKPALEDLRQGLLPVLESFKVSFLSALEEYTKKLNTQ

SEQ ID NO: 26 - protein, 243 aa, Human
DEPPQSPFDRVKDLATVYVDVLKDSGRDYVSQFEGSALGKQLNLKLLDNWDSVTSTFSKLREQLGPVTQEFWDNLEKETEGLRQEMSKDLEEVKAKVQPYLDDFQKKFQEEMELYRQKVEPLRAELQEGARQKLHELQEKLSPLGEEMRDCARAHVDALRTHLAPYSDELRQRLAARLEALKENGGARLAEYHAKATEHLSTLSEKAKPALEDLRQGLLPVLESFKVSFLSALEEYTKKLNTQ

SEQ ID NO: 27 - protein, 243 aa, Human
DEPPQSPWDRVKDLATVYVDVLKDSGRDYVSQFEGSALGKQLNLKLLDNFDSVTSTFSKLREQLGPVTQEFFDNLEKETEGLRQEMSKDLEEVKAKVQPYLDDFQKKWQEEMELYRQKVEPLRAELQEGARQKLHELQEKLSPLGEEMRDCARAHVDALRTHLAPYSDELRQRLAARLEALKENGGARLAEYHAKATEHLSTLSEKAKPALEDLRQGLLPVLESFKVSFLSALEEYTKKLNTQ

SEQ ID NO: 28 - protein, 243 aa, Human
DEPPQSPWDRVKDLATVYVDVLKDSGRDYVSQFEGSALGKQLNLKLLDNFDSVTSTFSKLREQLGPVTQEFWDNLEKETEGLRQEMSKDLEEVKAKVQPYLDDFQKKFQEEMELYRQKVEPLRAELQEGARQKLHELQEKLSPLGEEMRDCARAHVDALRTHLAPYSDELRQRLAARLEALKENGGARLAEYHAKATEHLSTLSEKAKPALEDLRQGLLPVLESFKVSFLSALEEYTKKLNTQ

SEQ ID NO: 29 - protein, 243 aa, Human
DEPPQSPWDRVKDLATVYVDVLKDSGRDYVSQFEGSALGKQLNLKLLDNWDSVTSTFSKLREQLGPVTQEFFDNLEKETEGLRQEMSKDLEEVKAKVQPYLDDFQKKFQEEMELYRQKVEPLRAELQEGARQKLHELQEKLSPLGEEMRDCARAHVDALRTHLAPYSDELRQRLAARLEALKENGGARLAEYHAKATEHLSTLSEKAKPALEDLRQGLLPVLESFKVSFLSALEEYTKKLNTQ

SEQ ID NO: 30 - protein, 243 aa, Human
DEPPQSPFDRVKDLATVYVDVLKDSGRDYVSQFEGSALGKQLNLKLLDNFDSVTSTFSKLREQLGPVTQEFFDNLEKETEGLRQEMSKDLEEVKAKVQPYLDDFQKKWQEEMELYRQKVEPLRAELQEGARQKLHELQEKLSPLGEEMRDCARAHVDALRTHLAPYSDELRQRLAARLEALKENGGARLAEYHAKATEHLSTLSEKAKPALEDLRQGLLPVLESFKVSFLSALEEYTKKLNTQ

SEQ ID NO: 31 - protein, 243 aa, Human
DEPPQSPFDRVKDLATVYVDVLKDSGRDYVSQFEGSALGKQLNLKLLDNWDSVTSTFSKLREQLGPVTQEFFDNLEKETEGLRQEMSKDLEEVKAKVQPYLDDFQKKFQEEMELYRQKVEPLRAELQEGARQKLHELQEKLSPLGEEMRDCARAHVDALRTHLAPYSDELRQRLAARLEALKENGGARLAEYHAKATEHLSTLSEKAKPALEDLRQGLLPVLESFKVSFLSALEEYTKKLNTQ

SEQ ID NO: 32 - protein, 243 aa, Human
DEPPQSPFDRVKDLATVYVDVLKDSGRDYVSQFEGSALGKQLNLKLLDNFDSVTSTFSKLREQLGPVTQEFWDNLEKETEGLRQEMSKDLEEVKAKVQPYLDDFQKKFQEEMELYRQKVEPLRAELQEGARQKLHELQEKLSPLGEEMRDCARAHVDALRTHLAPYSDELRQRLAARLEALKENGGARLAEYHAKATEHLSTLSEKAKPALEDLRQGLLPVLESFKVSFLSALEEYTKKLNTQ

SEQ ID NO: 33 - protein, 243 aa, Human
DEPPQSPWDRVKDLATVYVDVLKDSGRDYVSQFEGSALGKQLNLKLLDNFDSVTSTFSKLREQLGPVTQEFFDNLEKETEGLRQEMSKDLEEVKAKVQPYLDDFQKKFQEEMELYRQKVEPLRAELQEGARQKLHELQEKLSPLGEEMRDCARAHVDALRTHLAPYSDELRQRLAARLEALKENGGARLAEYHAKATEHLSTLSEKAKPALEDLRQGLLPVLESFKVSFLSALEEYTKKLNTQ

SEQ ID NO: 34 - DNA, 732 bp, Human
gatgaacccccccagagccccttcgatcgagtgaaggacctggccactgtgtacgtggatgtgctcaaagacagcggcagagactatgtgtcccagtttgaaggctccgccttgggaaaacagctaaacctaaagctccttgacaacttcgacagcgtgacctccaccttcagcaagctgcgcgaacagctcggccctgtgacccaggagttcttcgataacctggaaaaggagacagacggcctgaggcaggagatgagcaaggatctggaggaggtgaaggccaaggtgcagccctacctggacgacttccagaagaagttccaggaggagatggagctctaccgccagaaggtggagccgctgcgcgcagagctccaagagggcgcccgccagaagctgcacgagctgcaagagaagctgagcccactgggcgaggagatgcgcgaccgcgctcgcgcccatgtggacgcgctgcgcacccatctggccccctacagcgacgagctgcgccagtgcttggccgcccgccttgaggctctcaaggagaacggcggcgccagactggccgagtaccacgccaaggccaccgagcatctgagcacactgagcgagaaggccaagcccgccctcgaggacctccgccaaggcctgctgcccgtgctggagagcttcaaggtcagcttcctgagcgctctcgaggagtacactaagaagctcaacacccagtga

SEQ ID NO: 35 - protein, 243 aa, Homo sapiens
  MISC_FEATURE (1)..(243): 4WF ApoAI Polypeptide Monomer
DEPPQSPFDRVKDLATVYVDVLKDSGRDYVSQFEGSALGKQLNLKLLDNFDSVTSTFSKLREQLGPVTQEFFDNLEKETEGLRQEMSKDLEEVKAKVQPYLDDFQKKFQEEMELYRQKVEPLRAELQEGARQKLHELQEKLSPLGEEMRDRARAHVDALRTHLAPYSDELRQRLAARLEALKENGGARLAEYHAKATEHLSTLSEKAKPALEDLRQGLLPVLESFKVSFLSALEEYTKKLNTQ

SEQ ID NO: 36 - protein, 100 aa, Homo sapiens
  MISC_FEATURE (1)..(243): apolipoprotein A-II preproprotein [Homo sapiens].
MKLLAATVLLLTICSLEGALVRRQAKEPCVESLVSQYFQTVTDYGKDLMEKVKSPELQAEAKSYFEKSKEQLTPLIKKAGTELVNFLSYFVELGTQPATQ